(19) United States
(12) Patent Application Publication
(10) Pub. No.: US 2021/0206791 A1
(43) Pub. Date: Jul. 8, 2021

(54) SELECTIVE METAL-MEDIATED ARYLATION OF DICHALCOGENIDES IN BIOMOLECULES

(71) Applicant: Massachusetts Institute of Technology, Cambridge, MA (US)

(72) Inventors: Stephen L. Buchwald, Newton, MA (US); Bradley L Pentelute, Cambridge, MA (US); Chi Zhang, Cambridge, MA (US)

(21) Appl. No.: 16/081,370

(22) PCT Filed: Mar. 2, 2017
(86) PCT No.: PCT/US2017/020438

§ 371 Date: Aug. 30, 2018

(51) Int. Cl. C07F 15/00; C07J 43/00; C07J 51/00; C07K 1/00; C07K 5/062

(52) U.S. Cl. CPC C07F 15/0066; C07J 43/003; C07K 5/06034; C07K 1/006; C07J 51/00

(57) ABSTRACT

Disclosed are methods of selective cysteine and selenocysteine modification on peptide/protein molecules under physiologically relevant conditions. The methods feature several advantages over existing methods of peptide modification, such as specificity towards thiols and selenols over other nucleophiles (e.g., amines, hydroxyls), excellent functional group tolerance, and mild reaction conditions, including completely aqueous reaction conditions. Also disclosed are methods of preparing palladium complexes in the presence of oxygen.

### RELATED APPLICATIONS

This application claims the benefit of priority to U.S. Provisional Patent Application Ser. No. 62/302,357, filed Mar. 2, 2016, the contents of which are hereby incorporated by reference.

### GOVERNMENT SUPPORT

This invention was made with Government support under Grant Nos. GM046059, GM058160, and GM110535 awarded by the National Institutes of Health. The Government has certain rights in the invention.

### BACKGROUND

The ability to chemically modify amino acids allows for enrichment of biological understanding, synthesis of new protein-drug conjugates, improvement in nanomedicine, and exploration of functional hybrid materials. The so-called ‘bioconjugation’ reactions that are used most often are chemoselective, proceed at a high rate under mild reaction conditions (i.e., aqueous solvents/buffers, pH 6-8, temperature <37° C.), and are tolerant to the functional group diversity present in complex biomolecules. In addition, bioconjugation reactions should produce stable products and be used to modify peptides and proteins in a modular way, granting advanced structural diversity. The preparation of protein bioconjugates dates back over a century; however, few reports exist which meet all of these specifications.

A variety of bioconjugation methods have been developed, with each displaying several strengths and weaknesses. For example, N-hydroxysuccinimide (NHS) esters are commonly used for protein modification but are not compatible with amine-reactive buffers and tend to react with low chemoselectivity with the multitude of nucleophiles found in biomolecules. Click-chemistry relies on the introduction of unnatural amino acids, and maleimide technology leads to unstable products. Therefore, there exists a need to develop methods of cysteine functionalization, particularly methods that can tolerate various functional groups, reaction conditions, and that can generate stable products.

### SUMMARY

In one aspect, provided herein are methods of functionalizing a thiol or a selenol in a biopolymer, comprising:

contacting a biopolymer comprising a thiol or selenol moiety with a reagent of structural formula II, thereby generating a functionalized biopolymer, wherein the thiol or selenol moiety has been transformed to —S—Ar¹ or —Se—Ar¹:

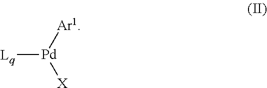

In another aspect, provided herein are methods of functionalizing a thiol or a selenol, wherein said method is represented by Scheme 1:

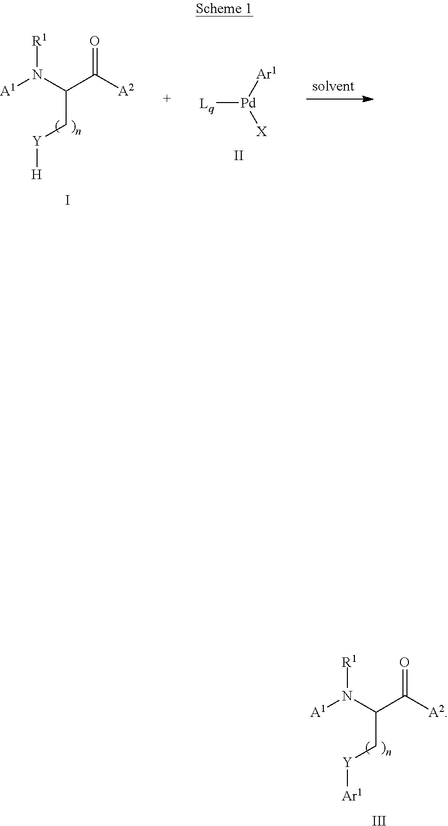

In another aspect, provided herein are methods comprising:

contacting a biopolymer comprising a first thiol moiety or a first selenol moiety and a second thiol or a second selenol moiety with a reagent of formula IV, thereby generating a functionalized biopolymer, wherein the first thiol moiety or the first selenol moiety has been covalently bound to the second thiol moiety or the second selenol moiety by —R^{y}:

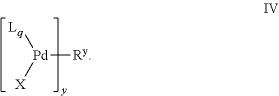

In another aspect, provided herein are methods, wherein said method is represented by Scheme 2:

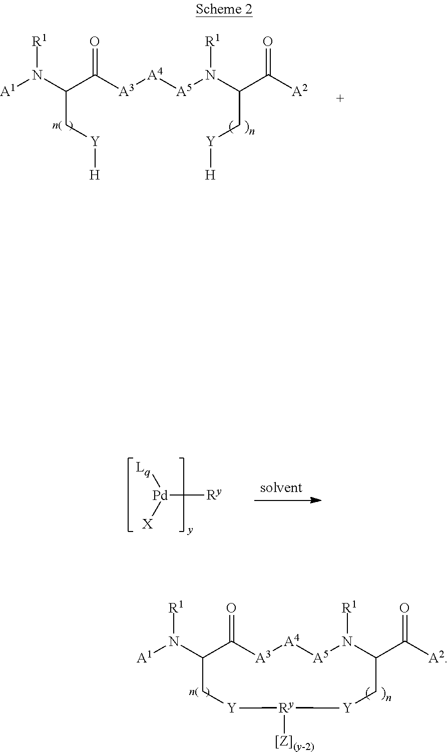

In another aspect, provided herein are methods according to Scheme 3:

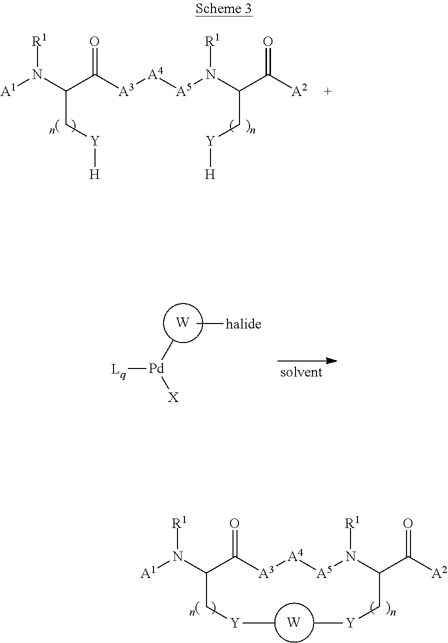

In another aspect, provided herein are methods preparing a palladium complex, wherein said method is represented by Scheme 4:

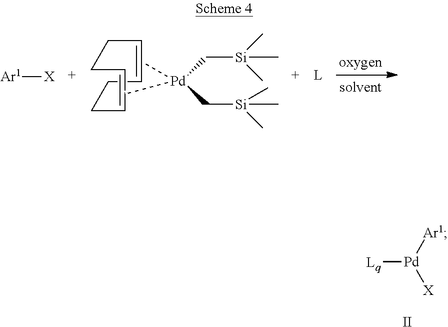

or represented by Scheme 5:

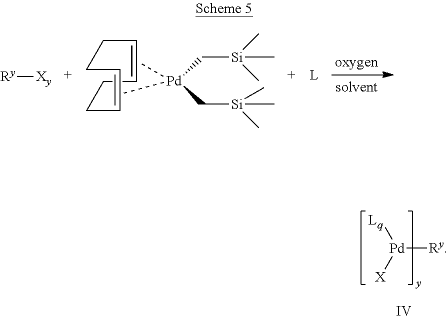

### BRIEF DESCRIPTION OF THE FIGURES

FIG. 1 depicts exemplary ligands (e.g., RuPhos=L1; SPhos=L2; and sSPhos=L3) useful in the invention.

FIG. 2 depicts a representative synthesis of exemplary monopailadium complexes of the disclosure.

FIG. 3 depicts a representative synthesis of a Pd-based reagent for cysteine and seienocysteme arylation for forming a drug conjugate.

FIG. 4 depicts exemplary pharmaceutical agents suitable for bioconjugation using Pd-based reagents.

FIG. 5 depicts a representative synthesis of Pd-based reagents comprising biotin.

FIG. 6A depicts a scheme for arylation of Cys-containing peptides using Pd(II) reagents under aqueous conditions.

FIG. 6B depicts an alternative scheme for arylation of Cys-containing peptides using Pd(II) reagents under aqueous conditions.

FIG. 7 is an LCMS trace of products from arylation of Cys-containing peptides in aqueous media.

FIG. 8 depicts two exemplary strategies (denoted A and B) for arylation of Cys sidechains in antibodies using Pd-based reagents.

FIG. 9A depicts a Pd(II) reagent with an arylated MMAE derivative.

FIG. 9B depicts an exemplary antibody-drug conjugate based on an arylated MMAE derivative conjugated to trastuzumab.

FIG. 10A depicts a Pd(II) reagent with an arylated cleavable MMAE derivative.

FIG. 10B depicts an exemplary antibody-drug conjugate based on an arylated cleavable MMAE derivative conjugated to trastuzumab.

FIG. 11 depicts the mass spectrometry for an exemplary antibody-drug conjugate based on camptothecin.

FIG. 12 depicts an experimental scheme for camptothecin arylation of the anti body trastuzumab.

FIG. 13 depicts the stability of an S-aryl antibody-drug conjugate in vitro.

FIG. 14 depicts an exemplary synthesis of a bifunctional Pd-based reagent with a RuPhos ligand for tire formation of a cyclic or stapled peptide.

FIG. 15 depicts an exemplary synthesis of a bifunctional Pd-based reagent with a sSPhos ligand for the formation of a cyclic or stapled peptide in a cosolvent system.

FIG. 16 depicts a schematic of a representative procedure for synthesis of a stapled peptide using a Pd-based haloarylation reagent in a cosolvent system.

FIG. 17 depicts a schematic of a representative procedure for synthesis of exemplary stapled peptides using Pd-based haloarylation reagents in a cosolvent system.

FIG. 18 depicts a schematic of a representative procedure for synthesis of a stapled peptide using a Pd-based haloarylation reagent in water.

FIG. 19 depicts a schematic of a representative procedure for synthesis of exemplary stapled peptides using Pd-based haloarylation reagents in water.

FIG. 20 depicts a representative arylation of DARPin using Pd-based reagents.

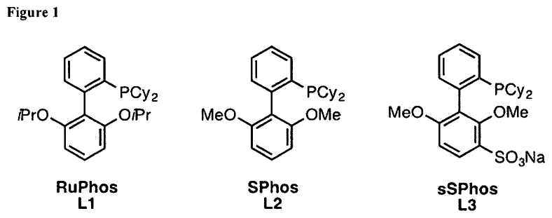

### DETAILED DESCRIPTION

Methods of selective cysteine and selenocysteine modification on peptide/protein molecules under physiologically relevant conditions are disclosed herein. The methods feature several advantages over existing methods of peptide modification, such as specificity towards thiols and selenols over other nucleophiles (e.g., amines, hydroxyls), excellent functional group tolerance, and mild reaction conditions, including completely aqueous reaction conditions. Also disclosed are methods of preparing palladium complexes in the presence of oxygen.

A palladium-mediated method was reported that can be used for the expeditious S-arylation of cysteines. This method is highly chemoselective for cysteine among other nucleophilic residues and can be used without incorporation of unnatural amino acids.¹¹ One notable limitation of this procedure, however, was the necessary addition of an organic cosolvent (N,N-dimethylformamide, dimethyl sulfoxide, or acetonitrile) to dissolve the organometallic reagents. Utilizing these solvents to perform chemical modification of biomolecules may lead to the denaturing of sensitive proteins or function inhibition, further complicating studies of the bioconjugates. Therefore, in some embodiments, disclosed herein are methods of selective cysteine and selenocysteine modification on peptide/protein molecules under completely aqueous reaction conditions.

### Exemplary Methods

In certain embodiments, the disclosure relates to a method of functionalizing a thiol or a selenol in a biopolymer, wherein the functionalization reagent is a compound of formula (IV) as described herein.

In one aspect, the disclosure relates to a method of functionalizing a thiol or a selenol in a biopolymer, comprising:

contacting a biopolymer comprising a thiol or selenol moiety with a reagent of structural formula II, thereby generating a functionalized biopolymer, wherein the thiol or selenol moiety has been transformed to —S—Ar¹ or —Se—Ar¹:

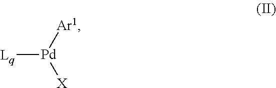

wherein

Ar¹ is selected from the group consisting of Ar¹ is selected from the group consisting of

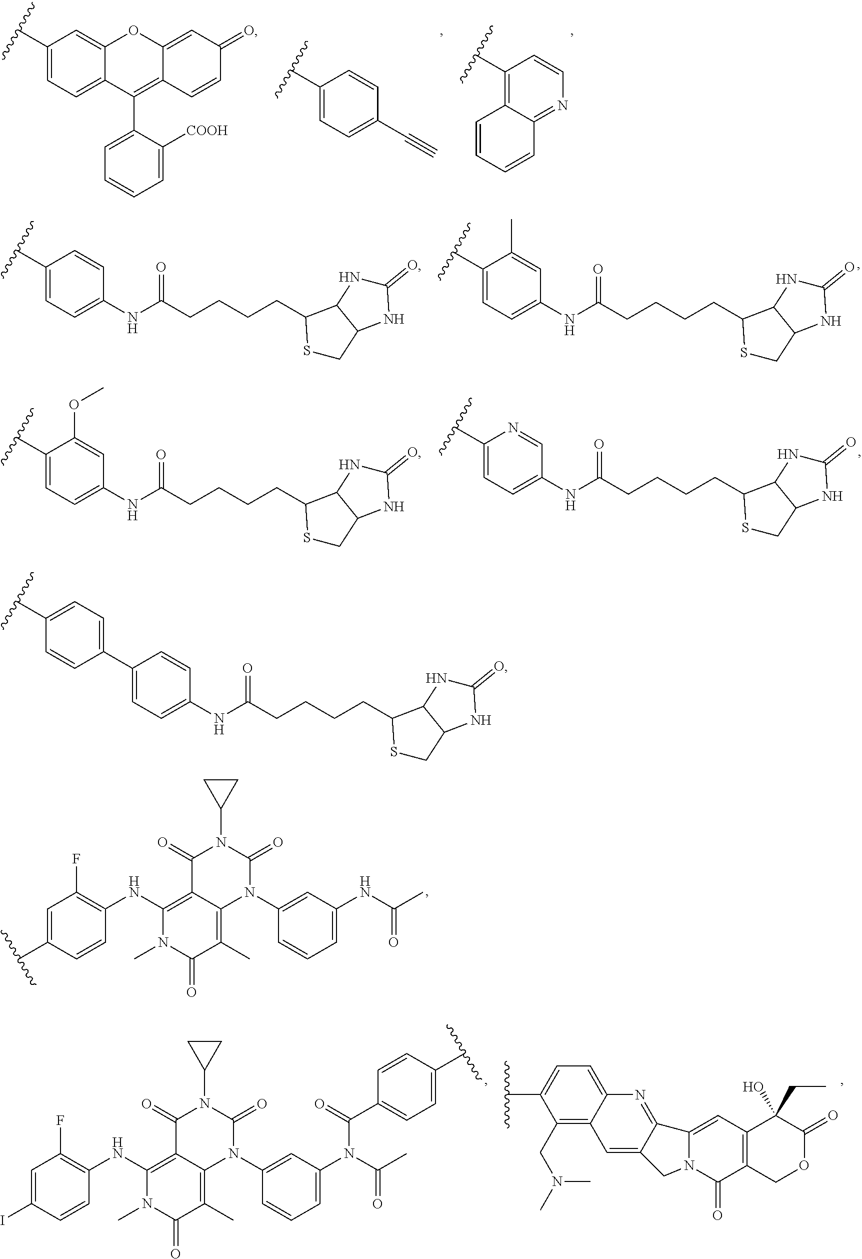

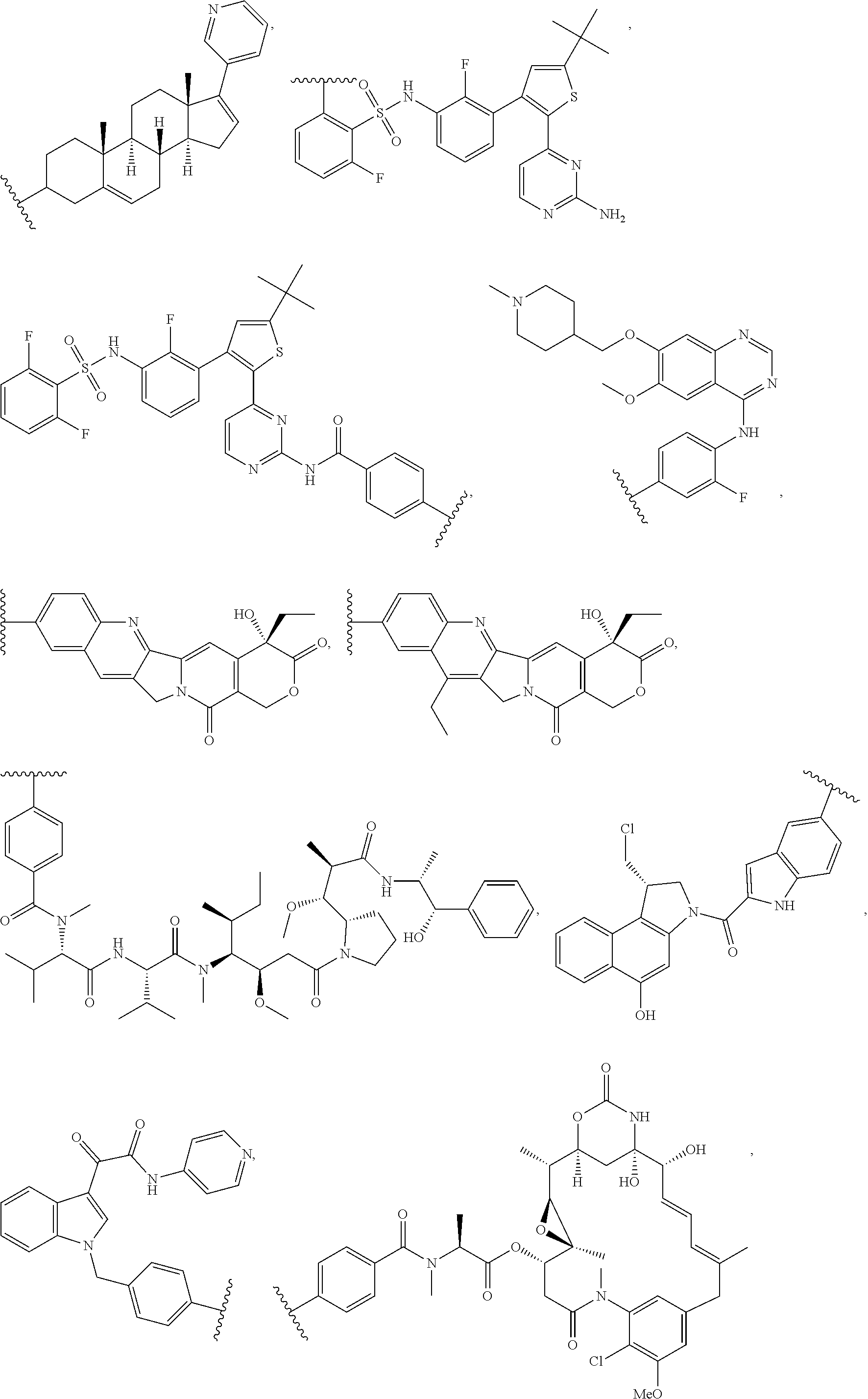

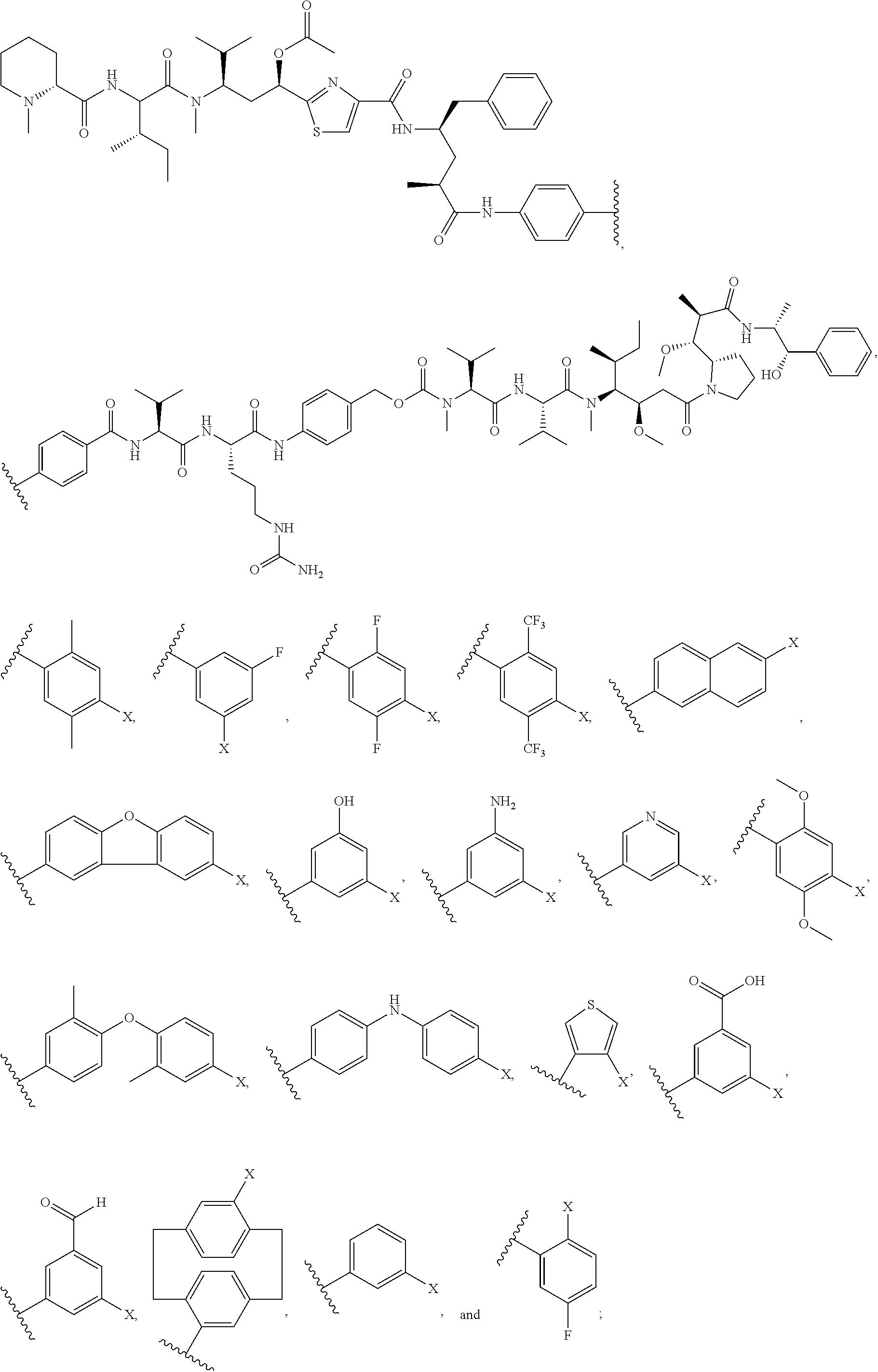

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyidialkylphosphine, a phosphine oxide, a bis(phosphine), a phosphoramide, atriarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine; and

q is 1 or 2.

In another aspect, the disclosure relates to a method of functionalizing a thiol or a selenol, wherein said method is represented by Scheme 1:

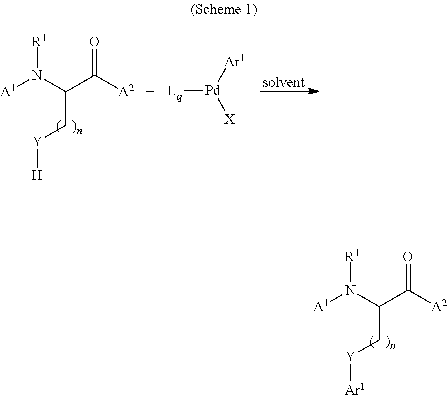

wherein:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

Ar¹ is optionally substituted aryl, heteroaryl, alkenyl, or cycloalkenyl;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

q is 1 or 2; and

solvent is water.

In some embodiments, provided herein are method of functionalizing a thiol or a selenol, wherein said method is represented by Scheme 1:

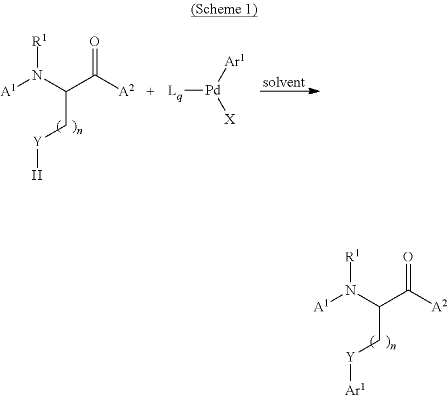

wherein:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

Ar¹ is selected from the group consisting of

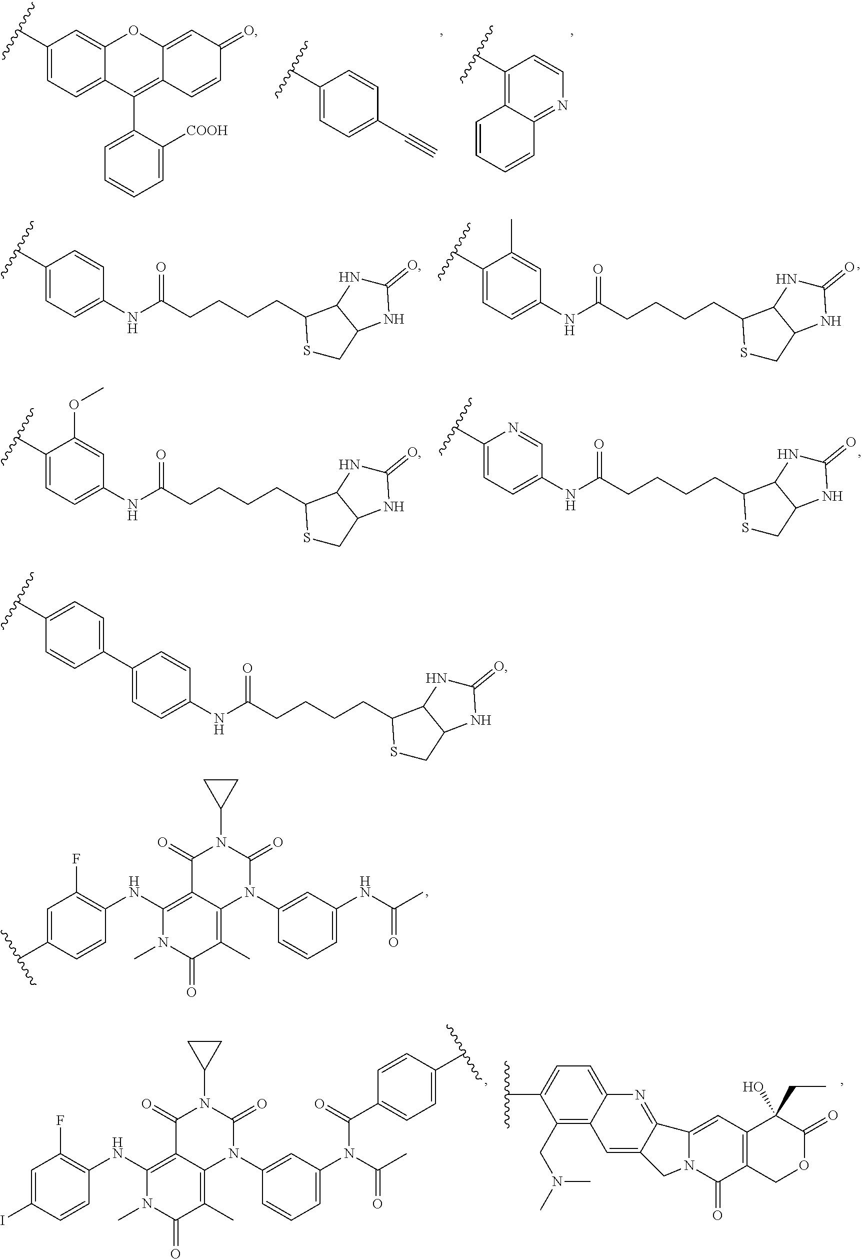

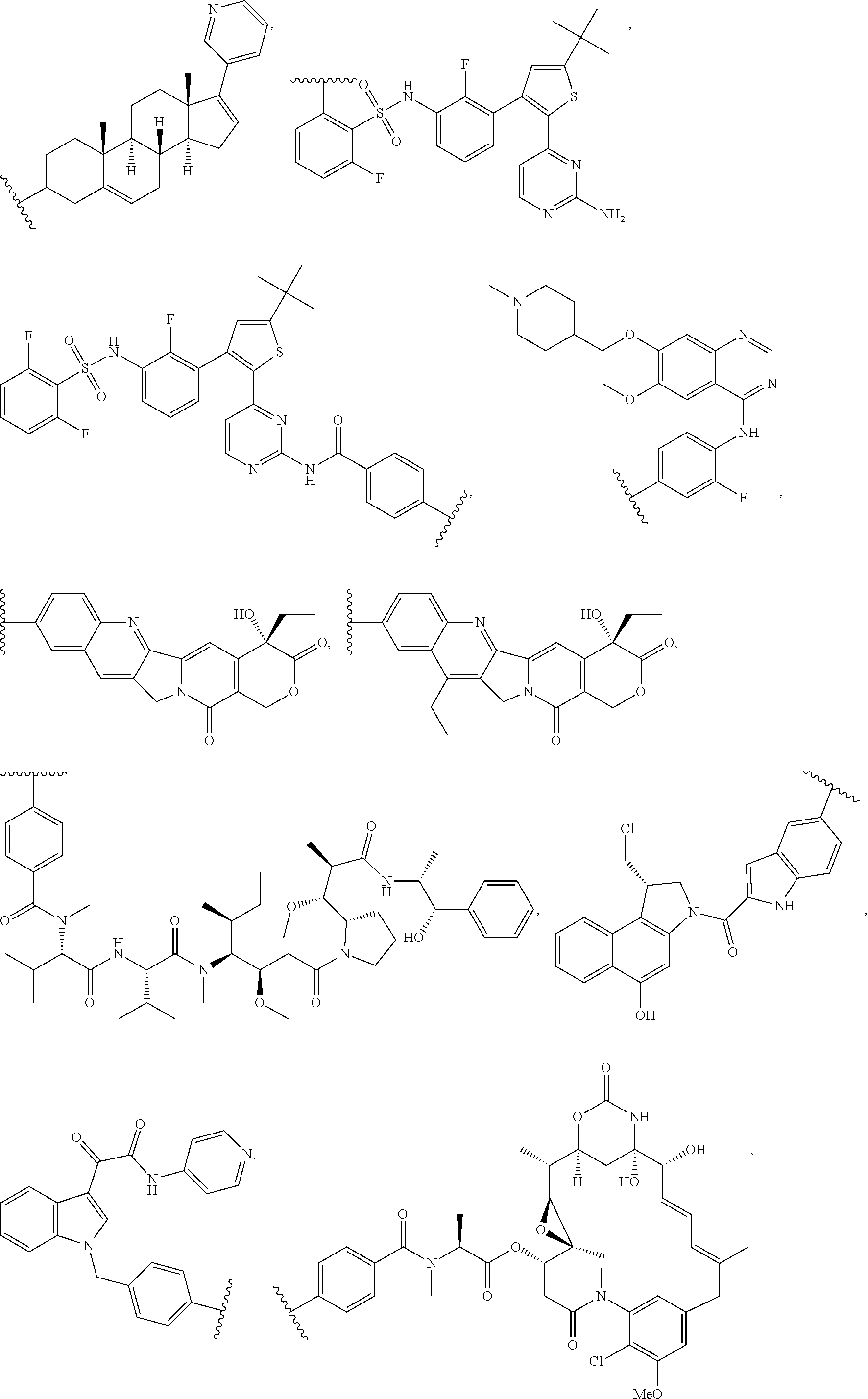

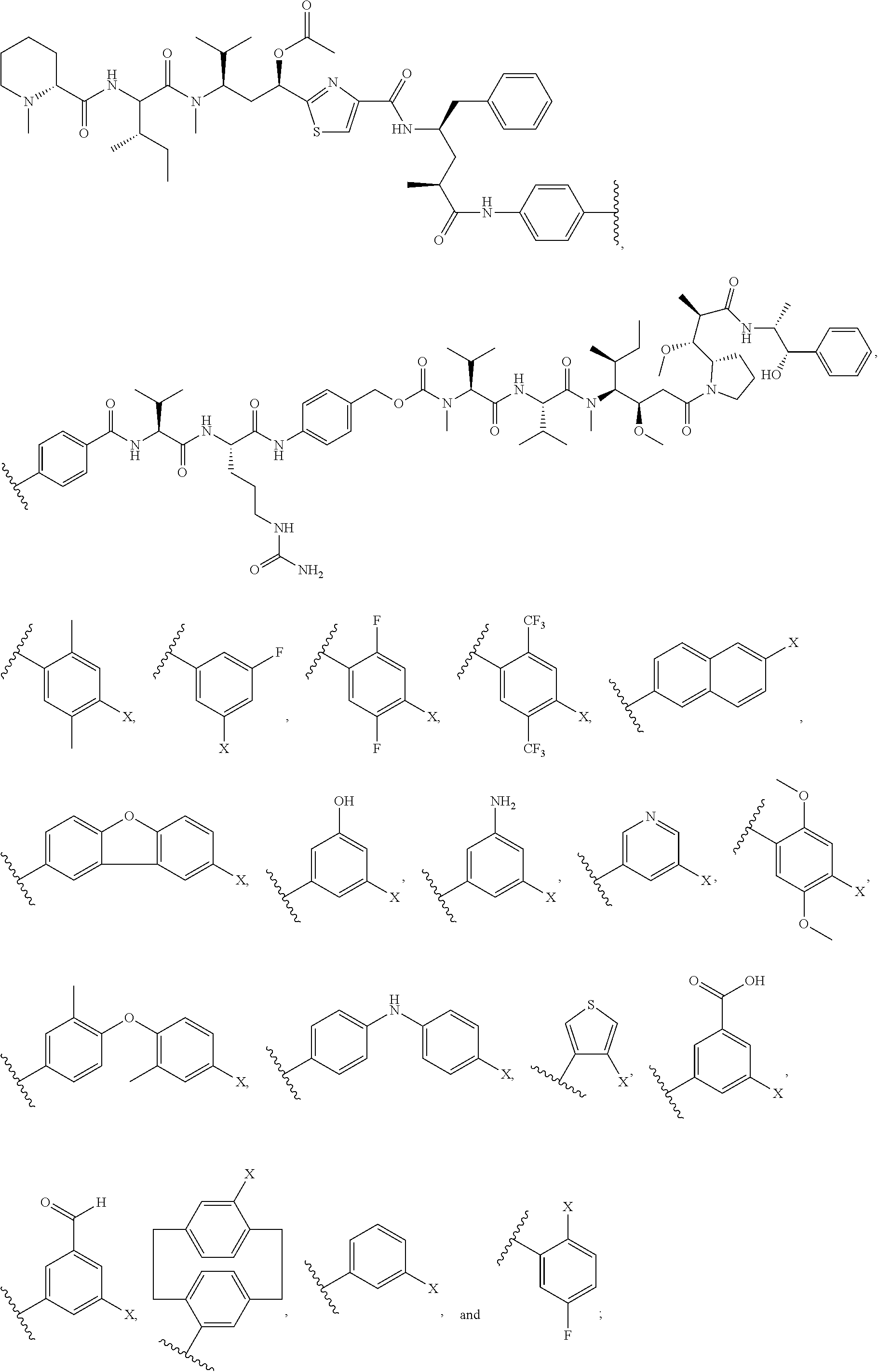

X is a halide, Inflate, tetrafluoroborate, tetraarylborate, hexafluoroantimonate, bis(alky 1 sulfonyl)amide, tetrafluorophosphate, hexafluorophosphate, alky 1 sulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, and sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine:

n is an integer from 1-5;

q is 1 or 2; and

solvent comprises water, and a polar protic solvent, a polar aprotic solvent, or a non-polar solvent.

The conditions under which the biopolymer and II come into contact with one another are sufficient to generate the functionalized biopolymer, in which Ar¹ is installed at the thiol or selenol moiety of the biopolymer. In certain embodiments, the biopolymer is an oligonucleotide, a polynucleotide, an oligosaccharide, or a polysaccharide.

In certain embodiments, the disclosure relates to a method of functionalizing a thiol or a selenol in a biopolymer, wherein the functionalization reagent is a compound of formula (II) as described herein.

Another aspect of the disclosure relates to a method, comprising contacting a biopolymer comprising a first thiol moiety or a first selenol moiety and a second thiol or a second selenol moiety with a reagent of formula IV as defined herein, thereby generating a functionalized biopolymer, wherein the first thiol moiety or the first selenol moiety has been co valently bound to the second thiol moiety or the second selenol moiety by R^{y}. The conditions under which the biopolymer and IV come into contact with one another are sufficient to generate the functionalized biopolymer. In certain embodiments, the biopolymer is an oligonucleotide, a polynucleotide, an oligosaccharide, or a polysaccharide.

In another aspect, the disclosure relates to a method, wherein said method is represented by Scheme 2:

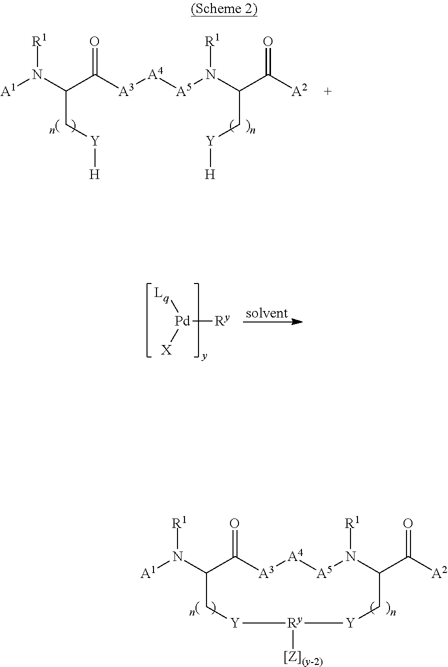

wherein:

A⁵ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment:

R^{y} is an optionally substituted bridging moiety, comprising an aromatic group, a heteroaromatic group, an alkene group, or a cycloalkene group;

y is 2, 3, 4, 5, or 6;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite-sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

q is 1 or 2;

each Z is independently

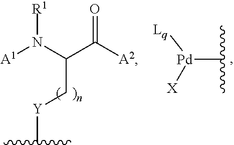

—S-alkyl, —SH, —S—(CH₂)ₙ—CO₂H, —SCH(CH₃)—CO₂H, or —SCH(CO₂H)—CH₂CO₂H. and

solvent is water.

In another aspect, provided herein is a method, wherein said method is represented by Scheme 2:

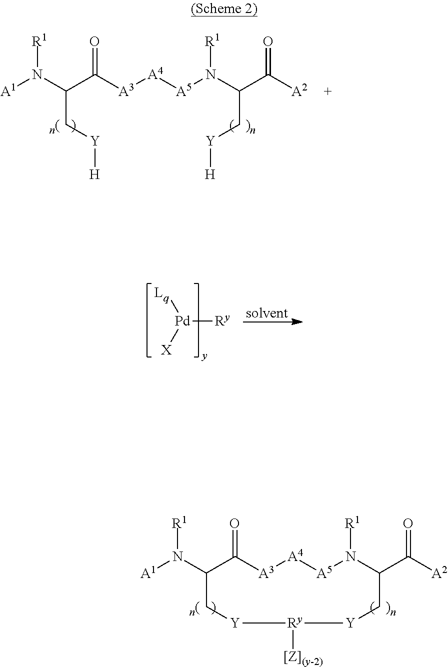

wherein:

A¹ is H, an amine protecting group, alkyl arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

R^{y} is selected from the group consisting of

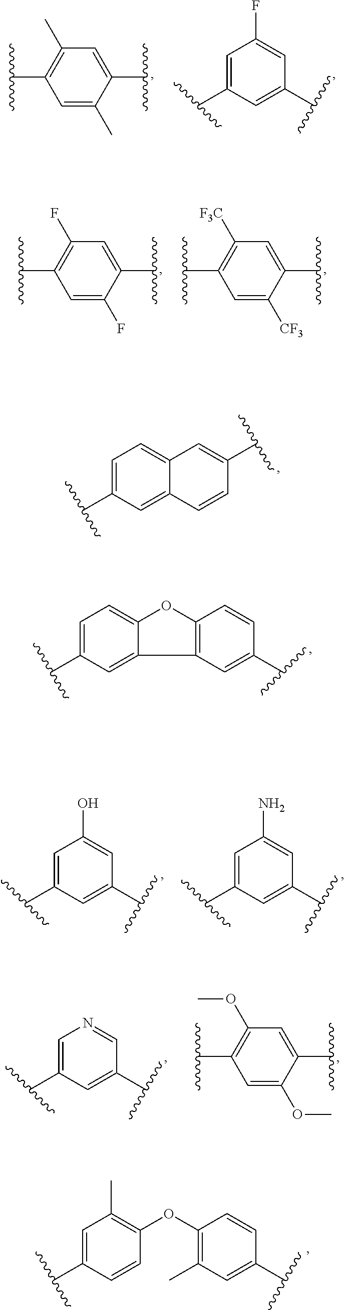

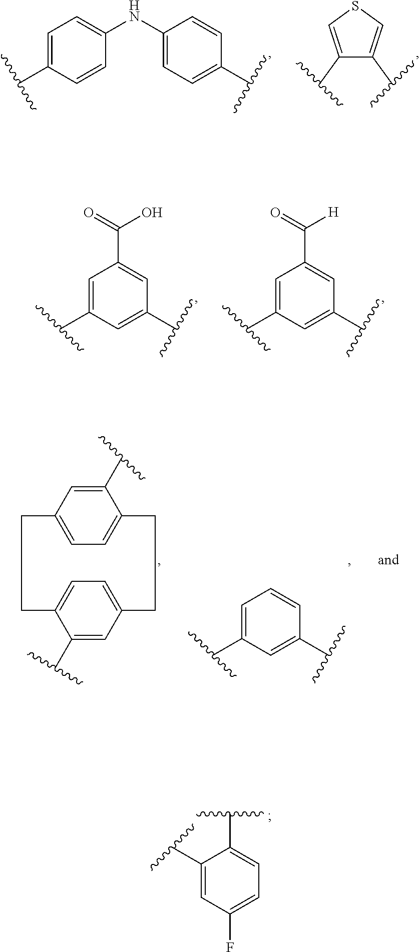

y is 2, 3, 4, 5, or 6;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

n is an integer from 1-5;

q is 1 or 2;

each Z is independently

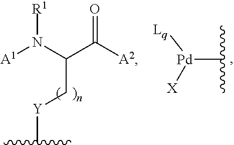

—S-alkyl, —SH, —S—(CH₂)ₙ—CO₂H, —SCH(CH₃)—CO₂H, or —SCH(CO₂H)—CH₂CO₂H; and

solvent comprises water, and a polar protic solvent, a polar aprotic solvent, or a non-polar solvent.

In another aspect, provided herein are methods comprising:

contacting a biopolymer comprising a first thiol moiety or a first selenol moiety and a second thiol or a second selenol moiety with a reagent of formula IV, thereby generating a functionalized biopolymer, wherein the first thiol moiety or the first selenol moiety has been covalently bound to the second thiol moiety or the second selenol moiety by —R^{y}:

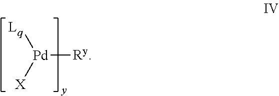

wherein, independently for each occurrence,

R^{y} is selected from the group consisting of

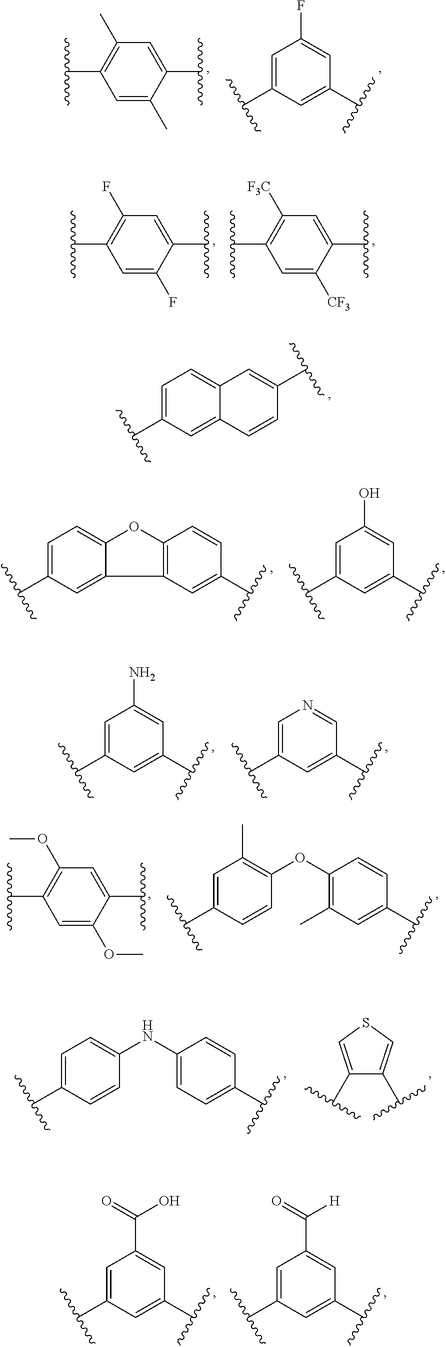

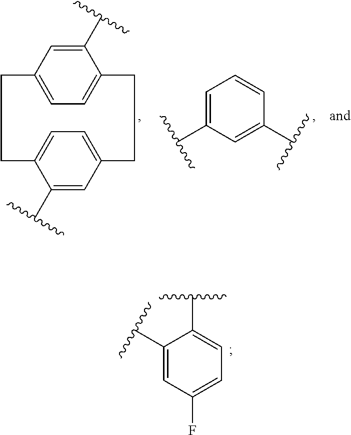

y is 2, 3, 4, 5, or 6:

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, and sulfate, carbonate, bicarbonate, carboxylate, phosphate, hydrogen phosphate, dihydrogen phosphate, phosphinate, or hypochlorite; L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine; and

q is 1 or 2,

The disclosure also provides methods for generating a stapled peptide using a mono-metallated catalyst bearing a haloaryl group. Such methods provide an alternative non-symmetric synthesis of a stapled peptide. For example, such synthesis can occur in a stepwise manner, in which a first bond forming step occurs between a first cysteine residue in a peptide and a mono-metallated haloarylation reagent. A second cross-coupling step may then occur between a second cysteine residue and the aryl halide, yielding the target stapled peptide product.

In another aspect, the disclosure relates to a method, wherein said method is represented by Scheme 3:

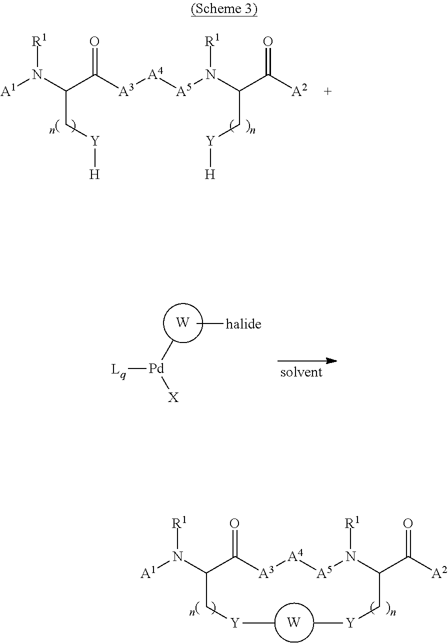

wherein:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

X is a halide, inflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

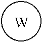

is and, heteroaryl, alkenyl, or cycloalkenyl, wherein

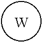

is optionally further substituted by one or more substituents selected from halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamide, heterocyclyl, aryl, heteroaryl, —CF₃, —CF₂R⁷, —CFR⁷₂, —CN, polyethylene glycol, polyethylene inline, —(CH₂)ₚ-FG-R⁷, and Z;

Z is

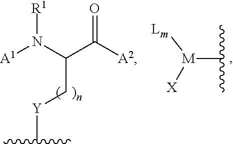

—S-alkyl, —SH, —S—(CH₂)ₙ—CO₂H, —SCH(CH₃)—CO₂H, or—SCH(CO₂H)—CH₂CO₂H;

p is independently for each occurrence an integer from 0-10;

FG is independently for each occurrence selected from the group consisting of C(O), CO₂, O(CO), ((O)NR⁷. NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl;

n is an integer from 1-5;

q is 1 or 2; and

solvent is water.

In another aspect, provided herein is a method, wherein said method is represented by Scheme 3:

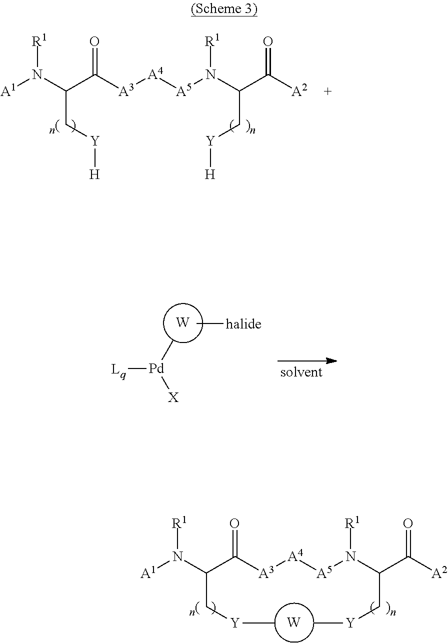

wherein:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyidialkylphosphine, a phosphine oxide, a bis(phosphine), a phosphoramide, atriarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

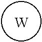

is selected from the group consisting of

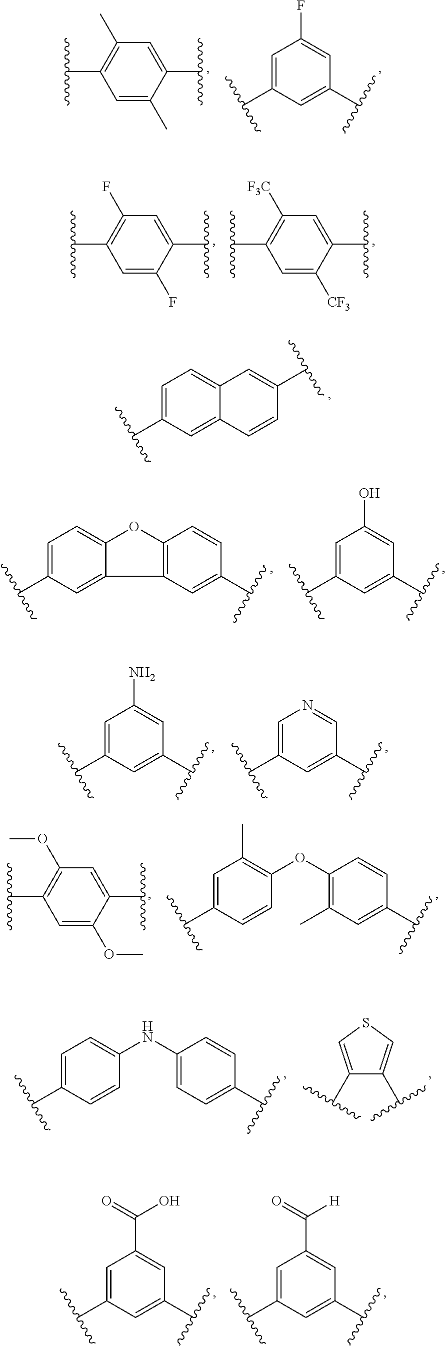

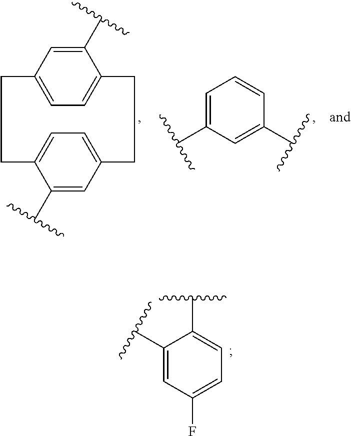

n is an integer from 1-5;

q is 1 or 2; and

solvent comprises water, and a polar protic solvent, a polar aprotic solvent, or a non-polar solvent.

In some embodiments of the methods disclosed herein, the disclosure relates to a method of functionalizing a thiol or a selenol in a biopolymer under aqueous reaction conditions. In some embodiments, the solvent is water. In some embodiments, the solvent is an aqueous buffer. In some embodiments, there is no cosolvent to water. In some embodiments, the solvent comprises water, and a polar protic solvent, a polar aprotic solvent, or a non-polar solvent.

In certain embodiments, the disclosure relates to any one of the aforementioned methods, wherein the solvent is an inert solvent, preferably one in which the reaction ingredients, including the catalyst, are substantially soluble. Suitable solvents include ethers such as diethyl ether, 1,2-dimethoxyethane, diglyme, t-butyl methyl ether, tetrahydrofuran, water and the like; halogenated solvents such as chloroform, dichloromethane, dichloroethane, chlorobenzene, and the like; aliphatic or aromatic hydrocarbon solvents such as benzene, xylene, toluene, hexane, pentane and the like; esters and ketones, such as ethyl acetate, acetone, and 2-butanone; polar aprotic solvents, such as acetonitrile, dimethylsulfoxide, dimethylformamide and the like; or combinations of two or more solvents.

In certain embodiments, the disclosure relates to any one of the aforementioned methods, wherein the solvent is a solvent mixture. In some embodiments, the solvent further comprises a polar protic solvent, a polar aprotic solvent, or a non-polar solvent (e.g., water and acetonitrile). In certain embodiments, the solvent mixture is an aqueous solvent mixture including a polar aprotic solvent. In certain embodiments, the disclosure relates to any one of the aforementioned methods, wherein the solvent comprises water and a polar protic solvent such as acetonitrile, dimethylsulfoxide, or dimethylformamide. In certain embodiments, the solvent is a solvent mixture comprising water and acetonitrile. In certain embodiments, the disclosure relates to any one of the aforementioned methods, wherein the solvent is a solvent mixture comprising water and dimethylformamide. In certain embodiments, the solvent mixture comprises from about 20:1 water to polar aprotic solvent to about 1:20 water to polar aprotic solvent, about 19:1 water to polar aprotic solvent to about 1:19 water to polar aprotic solvent, or about 18:1 water to polar aprotic solvent to about 1:18 water to polar aprotic solvent. In certain embodiments, the solvent mixture comprises from about 5:1 water to polar aprotic solvent to about 1:5 water to polar aprotic solvent. In certain embodiments, the solvent mixture further comprises a buffer. For example, the buffer may be Tris, HEPES, MOPS, MES, or Na₂HPO₄:NaH₂PO₄. In certain embodiments, the concentration of the buffer is from about 0.01 M to about 1 M, for example, about 25 mM or about 0.1 M.

In certain embodiments, the disclosure relates to any one of the aforementioned methods, wherein the reaction takes place at from about 4° C. to about 40° C. In certain embodiments, the disclosure relates to any one of the aforementioned methods, wherein the reaction takes place at about 10° C., about 15° C., about 20° C., about 25° C., about 30° C., or about 35° C.

In certain embodiments, the disclosure relates to any one of the aforementioned methods, wherein the reaction is substantially complete after about 10 s, about 20 s, about 30 s, about 40 s, about 50 s, about 1 min, about 2 min, about 3 min, about 4 min, about 5 min, about 10 min, about 15 min, about 20 min, about 25 min, about 30 min, about 35 min, about 40 min, about 45 min, about 50 min, about 55 min, about 60 min, about 65 min, about 70 min, about 75 min, about 80 min, about 85 min, or about 90 min. In certain embodiments, the disclosure relates to any one of tire aforementioned methods, wherein the reaction is substantially complete after about 2 h, about 3 h, about 4 h, about 5 h, about 6 h, about 7 h, about 8 h, about 9 h, about 10 h, about 11 h, or about 12 h.

The reactions of the present disclosure may be performed under a wide range of conditions, though it will be understood that the solvents and temperature ranges recited herein are not limitative and only correspond to exemplary modes of the processes of the disclosure.

In general, it will be desirable that reactions are run using mild conditions which will not adversely affect the reactants, the precatalyst, or the product. For example, the reaction temperature influences the speed of the reaction, as well as the stability of the reactants and catalyst. The reactions will usually be run at temperatures in the range of 20° C. to 300° C., more preferably in the range 20° C. to 150° C. In certain embodiments, the reactions will be ran at room temperature (i.e., about 20° C. to about 25° C.). In certain embodiments, the pH of the reaction mixture may be about 8.5. In certain embodiments, the pH of the reaction mixture may be about 8.0, about 7.5, about 7.0, about 6.5, about 6.0, about 5.5, about 5.0, about 4.5, about 4.0, about 3.5, about 3.0, about 2.5, about 2.0, or about 1.5.

In certain embodiments of the method represented by Scheme 1, Ar¹ is (C₆-C₁₀)carbocyclic aryl, (C₃-C₁₂)heteroaryl, (C₃-C₁₄)polycyclic aryl, or alkenyl; and Ar¹ is optionally substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamide, heterocyclyl, aryl, heteroaryl, —CF₃, —CF₂R⁷, —CFR⁷₂, —CN, polyethylene glycol, polyethylene imine, and —(CH₂)ₚ-FG-R⁷;

p is independently for each occurrence an integer from 0-10;

FG is independently for each occurrence selected from the group consisting of C(O), CO₂, O(CO), C(O)NR⁷, NR⁷C(O), O, Si(R⁷)₂—, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl; and

if two or more substituents are present on Ar¹, then two of said substituents taken together may form a ring;

wherein at least one of the one or more substituents is halide.

Certain arylated products contain functional groups that allow for further functionalization of the product. In certain embodiments, an aryl-halide bond provides a useful handle for such further functionalization. For example, the aryl-halide bond can undergo a metal-catalyzed or metal-mediated cross-coupling reaction with an additional thiol-containing reagent.

Accordingly, in certain embodiments wherein Ar¹ is (C₆-C₁₀)carbocyclic aryl, (C₃-C₁₂)heteroaryl, (C₃-C₁₄)polycyclic aryl, or alkenyl substituted by at least one halide, the method represented by Scheme 1 further comprises contacting compound III,

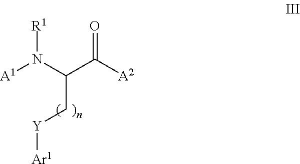

with a compound containing a thiol moiety or a selenol moiety; thereby yielding a coupling product.

In certain embodiments, the compound containing a thiol moiety or a selenol moiety is a small molecule having a molecular weight below about 500 g/mol.

In certain embodiments, the compound containing a thiol moiety or a selenol moiety is a biomolecule such as a natural or unnatural amino acid, a plurality of natural or unnatural amino acids, peptide, oligopeptide, polypeptide, or protein.

In certain embodiments, the step of contacting compound III with a compound containing a thiol moiety or a selenol moiety occurs in the presence of a Pd byproduct from the reaction depicted in Scheme 1.

### Exemplary Compounds

In certain embodiments, the disclosure relates to a compound comprising substructure III:

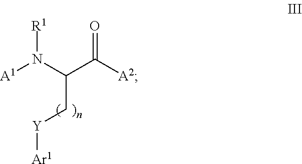

wherein,

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

Y is S or Se;

R¹ is H, alkyl, arylalkyl, acyl, aryl, alkoxycarbonyl, or aryloxycarbonyl, a natural or unnatural amino acid, a plurality of natural amino acids or unnatural amino acids, a peptide, an oligopeptide, a polypeptide, a protein, an antibody, or an antibody fragment;

n is an integer from 1-5; and

Ar¹ is optionally substituted aryl, heteroaryl, alkenyl, or cycloalkenyl.

### Aryl Definitions of the Compounds

In some embodiments of tire compounds disclosed herein, Ar¹ is covalently linked to a fluorophore, an imaging agent, a detection agent, a biomolecule, a therapeutic agent, a lipophilic moiety, a member of a high-affinity binding pair, or a cell-receptor targeting agent. In certain embodiments, the disclosure relates to any one of the aforementioned compounds, wherein Ar¹ is covalently linked to biotin. In certain embodiments, the disclosure relates to any one of the aforementioned compounds, wherein Ar¹ is covalently linked to fluorescein. In certain embodiments, the disclosure relates to any of the aforementioned compounds, wherein Ar¹ is covalently linked to a therapeutic agent; and the therapeutic agent is trametinib, topotecan, abiraterone, dabrafenib, vandetanib, camptothecin, SN-38, MMAE, duocarmycin SA, indibulin, tubulysin A, and maytansine.

In some embodiments of the compounds disclosed herein, Ar¹ is comprised by a fluorophore. In certain embodiments, the disclosure relates to any of the aforementioned compounds, wherein Ar¹ is comprised by a therapeutic agent. In certain embodiments, tire therapeutic agent is the trametinib, topotecan, abiraterone, dabrafenib, vandetanib, camptothecin, SN-38, MMAE, duocarmycin SA, indibulin, tubulysin A, and maytansine.

In certain embodiments, the fluorophore is a derivative of xanthene, fluorescein, rhodamine, coumarin, naphthalene, anathracene, oxadiazole, pyrene, acridine, tetrapyrrole, arylmethine, boron-dipyrromethene (BODIPY), or a cyanine dye. In certain other embodiments, the fluorophore is a fluorescent protein. In certain embodiments, the detection agent is for example, a nanoparticle, an MRI contrast agent, a dye moiety, or a radionuclide. In certain other embodiments, a biomolecule is a protein, a peptide, a monosaccharide, a disaccharide, a polysaccharide, a lipid, a glycolipid, a glycerolipid, a phospholipid, a hormone, a neurotransmitter, a nucleic acid, a nucleotide, a nucleoside, a sterol, a metabolite, a vitamin, or a natural product.

In certain embodiments, a therapeutic agent is a compound or substructure of a compound that brings about a therapeutic effect in a subject to which the agent is administered. In certain embodiments, the therapeutic agent is toxic to certain cells. Exemplary therapeutic agents that are covalently linked to Ar¹ in the compounds disclosed herein include trametinib, topotecan, abiraterone, dabrafenib, vandetanib, camptothecin, SN-38, MMAE, duocarmycin SA, indibulin, tubulysin A, and maytansine.

In some embodiments of the compounds disclosed herein, Ar¹ is selected from the group consisting of Ar¹ is selected from tire group consisting of

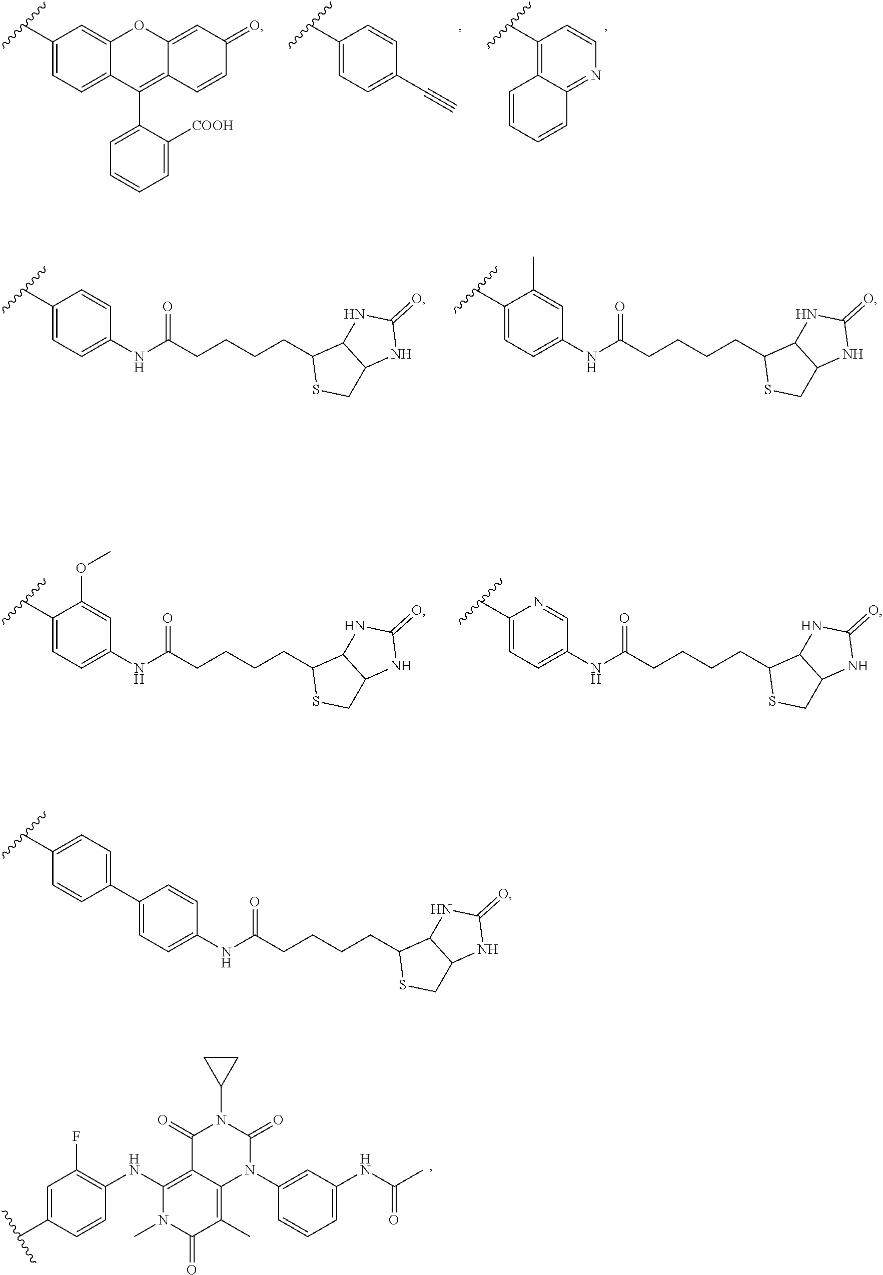

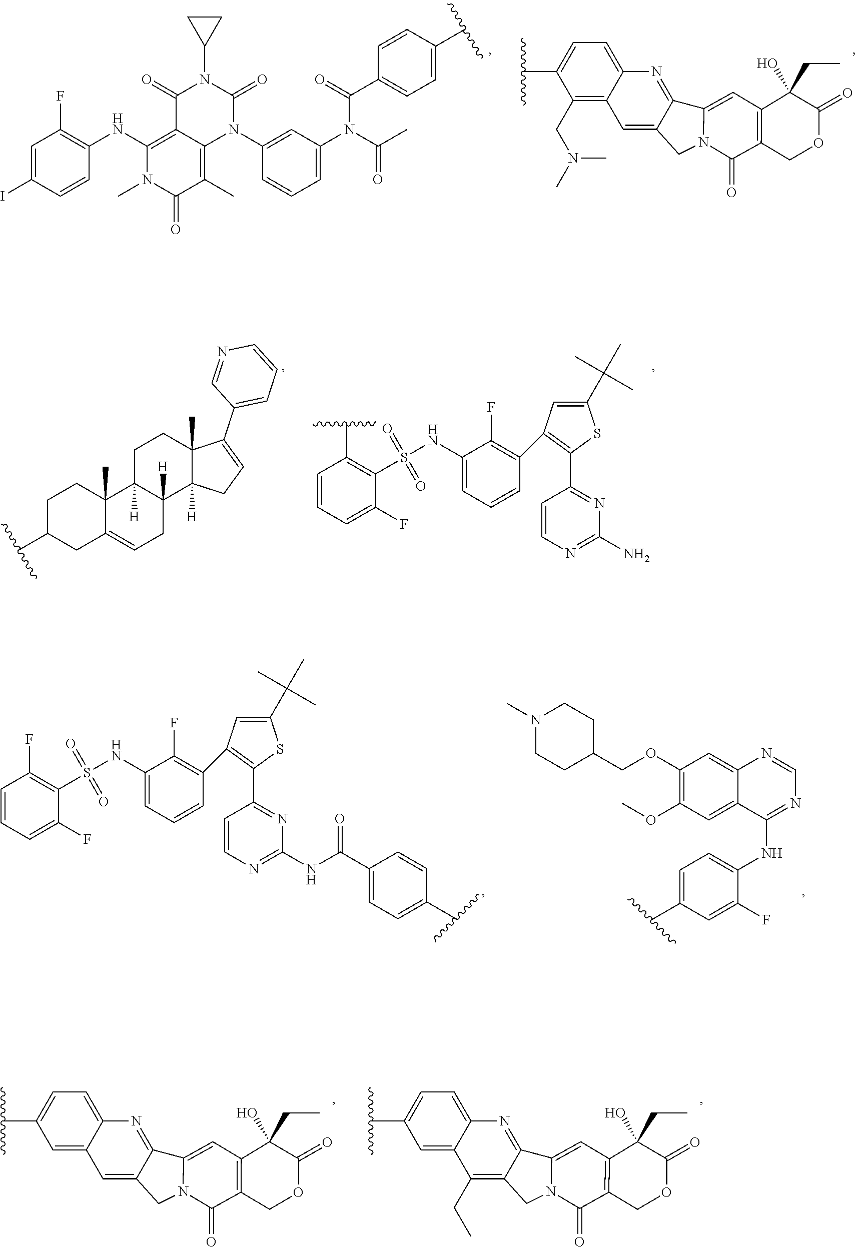

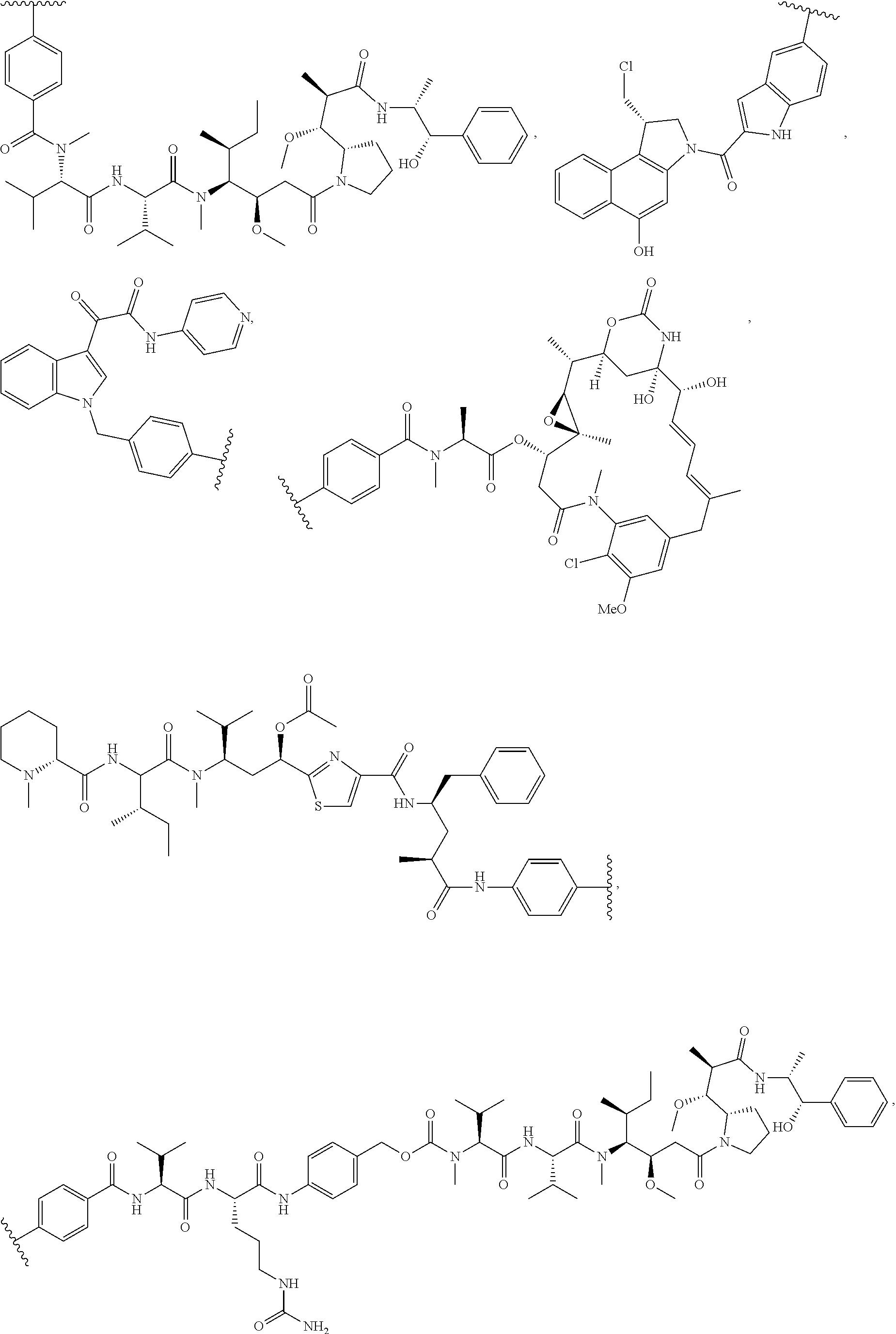

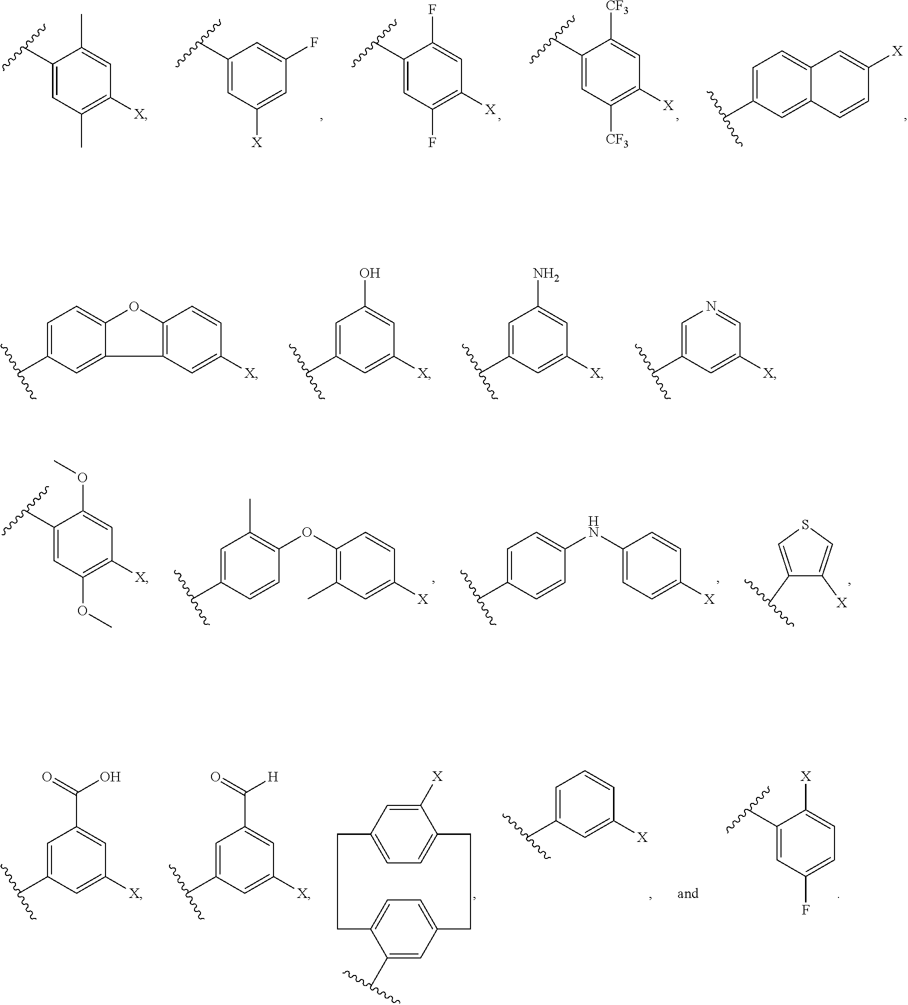

In some embodiments, Ar¹ is selected from the group consisting of

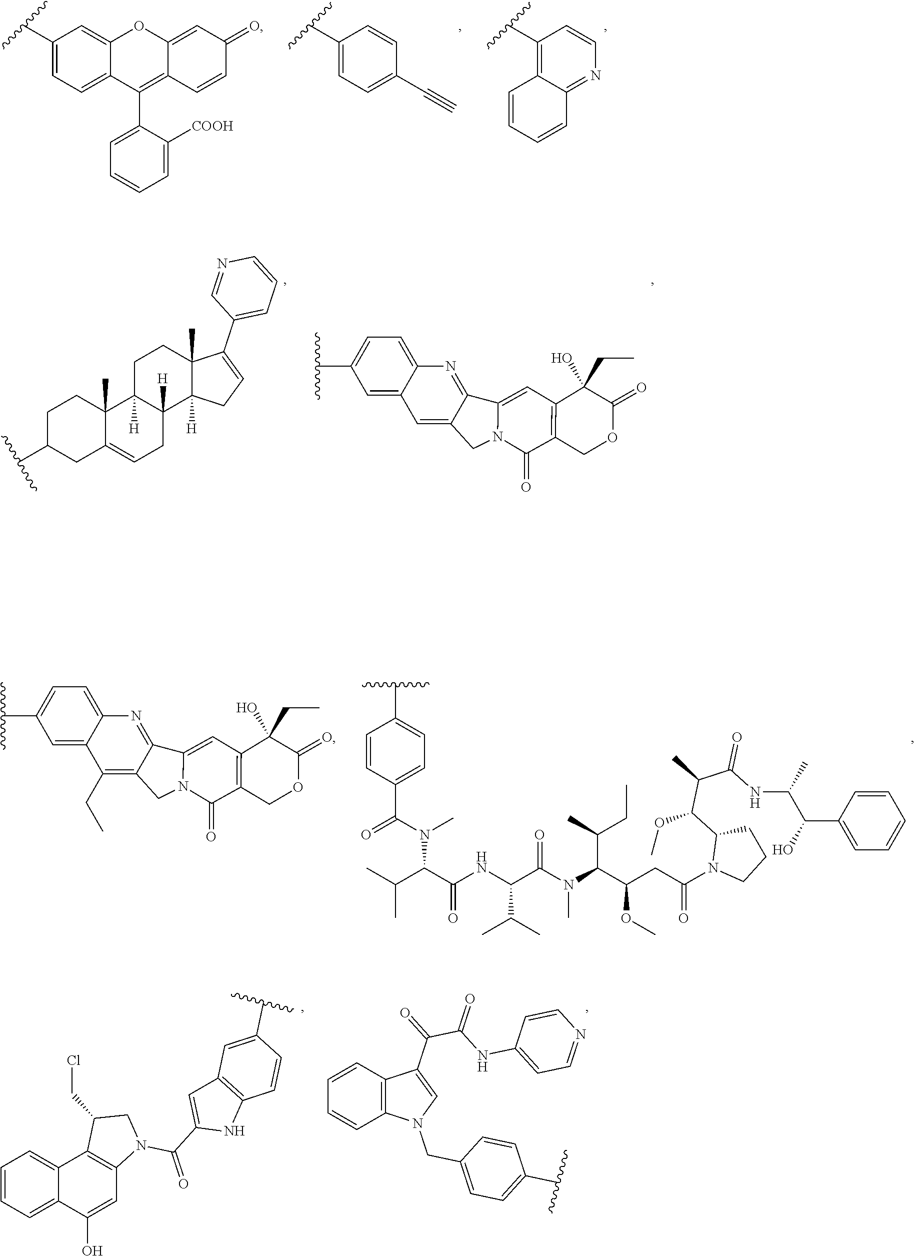

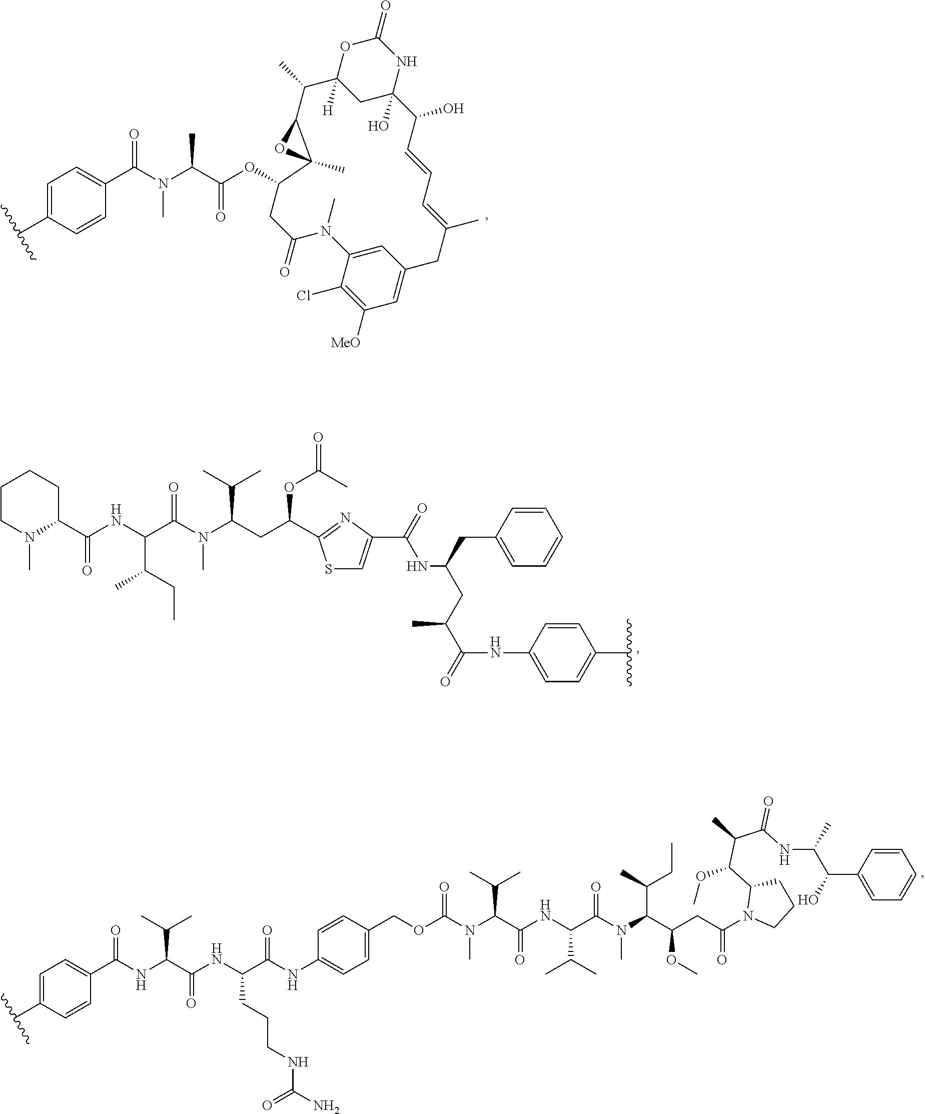 [SMILES: *c1ccc(C(=O)N(C)[C@@H](C)C(=O)O[C@H]2CC(=O)N(C)c3cc(cc(OC)c3Cl)C/C(C)=C/C=C/[C@@H](O)[C@@]3(O)C[C@H](OC(=O)N3)[C@@H](C)[C@@H]3O[C@]23C)cc1 ; *c1ccc(NC(=O)[C@@H](C)C[C@H](Cc2ccccc2)NC(=O)c2csc([C@@H](C[C@H](C(C)C)N(C)C(=O)C(NC(=O)[C@H]3CCCCN3C)[C@@H](C)CC)OC(C)=O)n2)cc1 ; *c1ccc(C(=O)N[C@H](C(=O)N[C@@H](CCCNC(N)=O)C(=O)Nc2ccc(COC(=O)N(C)[C@H](C(=O)N[C@H](C(=O)N(C)[C@@H]([C@@H](C)CC)[C@@H](CC(=O)N3CCC[C@H]3[C@H](OC)[C@@H](C)C(=O)N[C@H](C)[C@@H](O)c3ccccc3)OC)C(C)C)C(C)C)cc2)C(C)C)cc1]

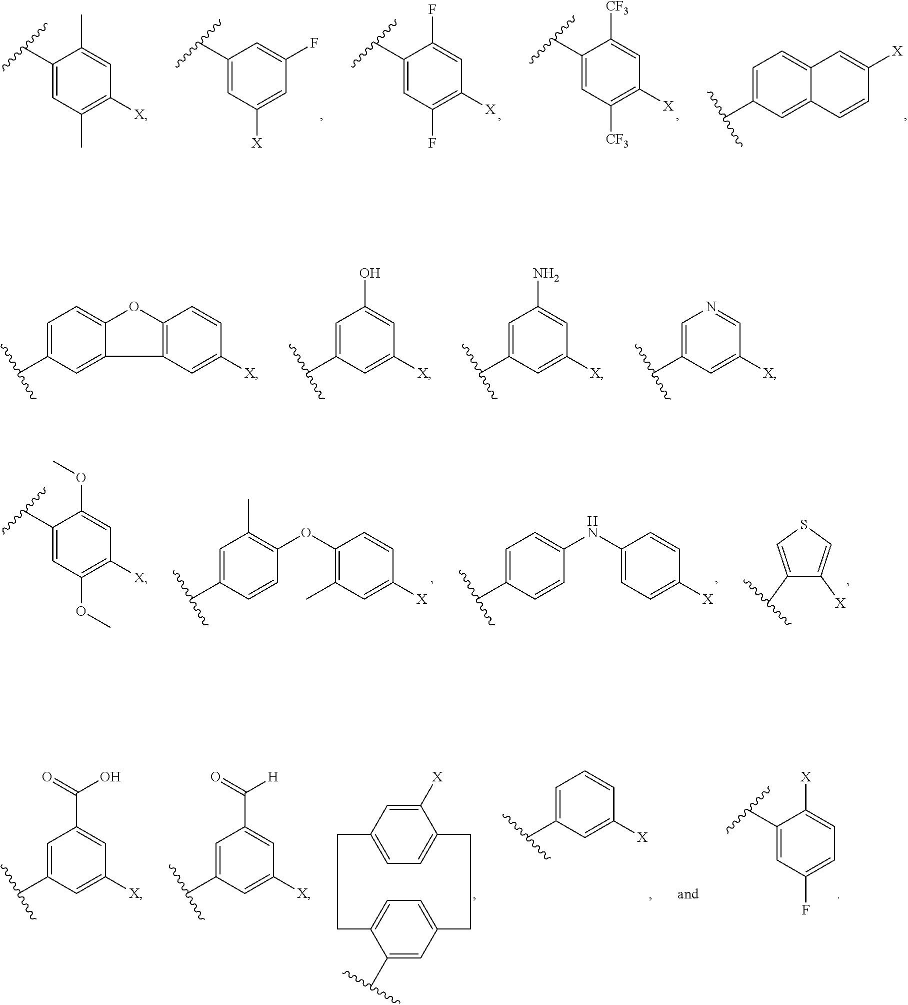

In certain embodiments disclosed herein, the lipophilic moiety enables the compounds disclosed herein to which the lipophilic moiety is conjugated to have an affinity for, or be soluble in, lipids, fats, oils, ad non-polar solvents, as described herein. Exemplary lipophilic moieties include amphilphilic surfactants, such as cinnamic acid.

In certain embodiments disclosed herein, the cell-receptor targeting agent is a ligand such as an epitope, a peptide, an antibody, a small organic compound, a neurotransmitter. High-affinity binding pairs include biotin-avidin, biotin-streptavidin, ligand-cell receptor, S-Peptide and Ribonuclease A, digoxigenin and its receptor, and complementary oligonucleotide pairs.

In some embodiments of the compounds disclosed herein, A¹ and A² are independently a natural or unnatural amino acid, a plurality of natural or unnatural amino acids, a peptide, an oligopeptide, a polypeptide, or a protein.

In some embodiments of the compounds disclosed herein, A¹ and A² each independently comprise arginine, histidine, lysine, aspartic acid, glutamic acid, serine, threonine, asparagine, glutamine, proline, tyrosine, or tryptophan. In certain embodiments, A¹ and A² do not comprise cysteine or selenocysteine. In certain embodiments, A¹ and A² do not comprise any amino acids that contain —SH or —SeH moieties.

In some embodiments of the compounds disclosed herein, R¹ is H. In some embodiments of the compounds disclosed herein, X is halide, such as chloride. In certain embodiments, X is inflate.

In some embodiments of the compounds disclosed herein, A¹ and A² are covalently linked. In some embodiments of the compounds disclosed herein, the compound comprises a cyclic peptide having an functionalized S moiety or a functionalized Se moiety. In certain embodiments, the functionalized S moiety or functionalized Se moiety is an arylated S moiety or an arylated Se moiety, respectively.

In certain embodiments, A¹ or A² comprises an antibody or an antibody fragment. In certain embodiments, the antibody is intact and comprises a single-point mutation with functionalized (e.g., arylated) Cys, Sec, or an artificial amino acid comprising —S(functional group) or —Se(functional group) on its main chain terminus. In alternative embodiments, A¹ or A² comprises an antibody fragment after partial antibody reduction.

### Exemplary Stapled Compounds

In certain embodiments, tire disclosure relates to a compound comprising substructure V:

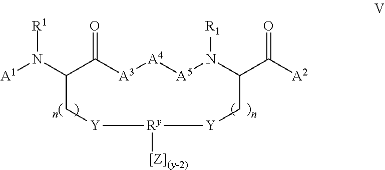

wherein, independently for each occurrence,

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A⁵ are selected from the group consisting of a natural amino acid, an unnatural amino acid, and a plurality of natural amino acids or unnatural amino acids;

Y is S or Se;

n is 1-5;

R^{y} is an optionally substituted bridging moiety, comprising an aromatic group, a heteroaromatic group, an alkene group, or a cycloalkene group;

y is 2, 3, 4, 5, or 6;

each Z is independently

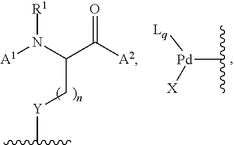

—S-alkyl, —SH, —S—(CH₂)ₙ—CO₂H, —SCH(CH₃)—CO₂H, or —SCH(CO₂H)—CH₂CO₂H; and

R¹ is H, alkyl, arylalkyl, acyl, aryl, alkoxycarbonyl, or aryloxycarbonyl, a natural or unnatural amino acid, a plurality of natural amino acids or unnatural amino acids, a peptide, an oligopeptide, a polypeptide, a protein, an antibody, or an antibody fragment.

In certain embodiments, the disclosure relates to any of the compounds described herein, wherein none of A¹, A², A³, A⁴, and A⁵ comprises cysteine.

In certain embodiments, the disclosure relates to any of the compounds described herein, wherein one or more of A¹, A², A³, A⁴, and A⁵ comprises arginine, histidine, lysine, aspartic acid, glutamic acid, serine, threonine, asparagine, glutamine, glycine, proline, alanine, valine, isoleucine, leucine, methionine, phenylalanine, tyrosine, or tryptophan.

In certain embodiments, the disclosure relates to any of the compounds described herein, wherein R^{y} is an optionally substituted bifunctional bridging moiety or an optionally substituted trifunctional bridging moiety.

In certain embodiments, the disclosure relates to any of the compounds described herein, wherein R^{y} comprises an aromatic group.

In certain embodiments, the disclosure relates to any of the compounds described herein, wherein R^{y} is optionally substituted

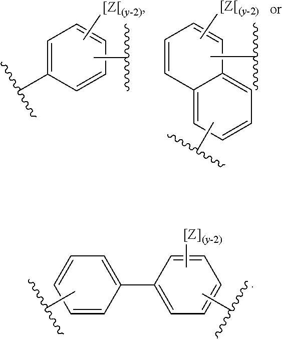

In certain embodiments, the disclosure relates to any of the compounds described herein, wherein R^{y} is not a perfluorinated aryl para-substituted diradical.

In certain embodiments, the disclosure relates to any one of the compounds described herein, wherein y is 2; and R^{y} is selected from the group consisting of

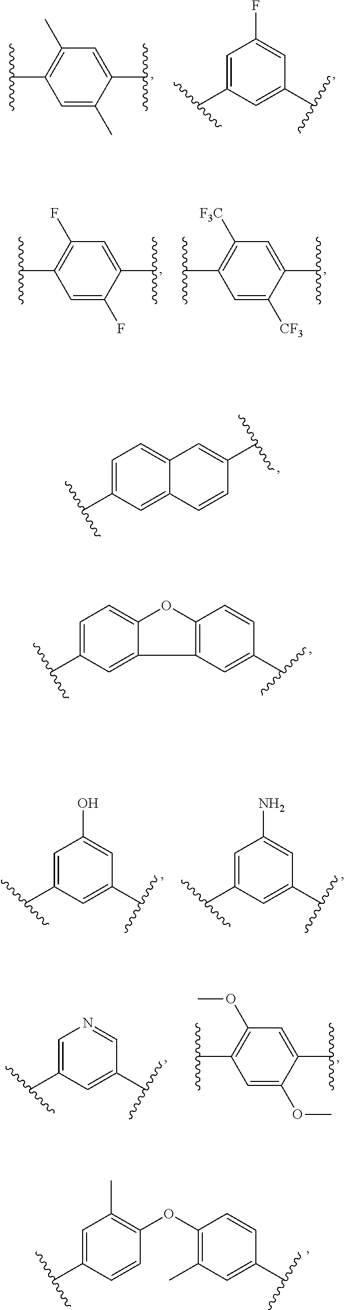

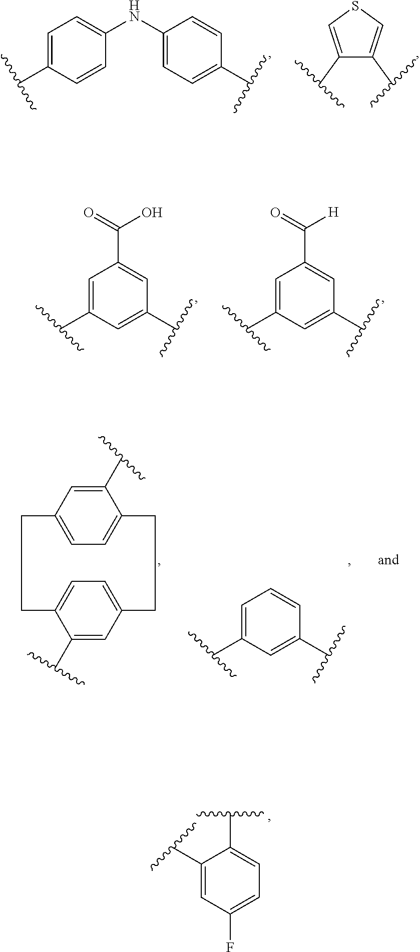

wherein any of the bifunctional bridging moieties may be optionally substituted.

In certain embodiments, the disclosure relates to a compound comprising substructure VI:

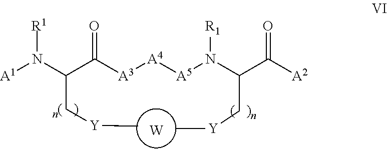

wherein, independently for each occurrence:

A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;

A³, A⁴, and A³ are selected from the group consisting of a natural amino add, an unnatural amino acid, and a plurality of natural amino acids or unnatural amino acids;

Y is S or Se;

R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;

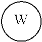

is aryl, heteroaryl, alkenyl, or cycloalkenyl, wherein

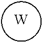

is optionally further substituted by one or more substituents selected from halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, —(CH₂)ₚ-FG-R⁷, and Z;

Z is

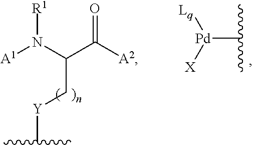

—S-alkyl, —SH, —S—(CH₂)ₙ—CO₂H, —SCH(CH₃)—CO₂H, or—SCH(CO₂H)—CH₂CO₂H;

p is independently for each occurrence an integer from 0-10;

FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);

R⁷ is independently for each occurrence selected from the group consisting of H, alkyl, cycloalkyl, aryl, aralkyl, alkenyl, and alkynyl;

n is an integer from 1-5; and

q is 1 or 2.

In certain embodiments of the compounds disclosed herein,

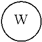

is selected from the group consisting of

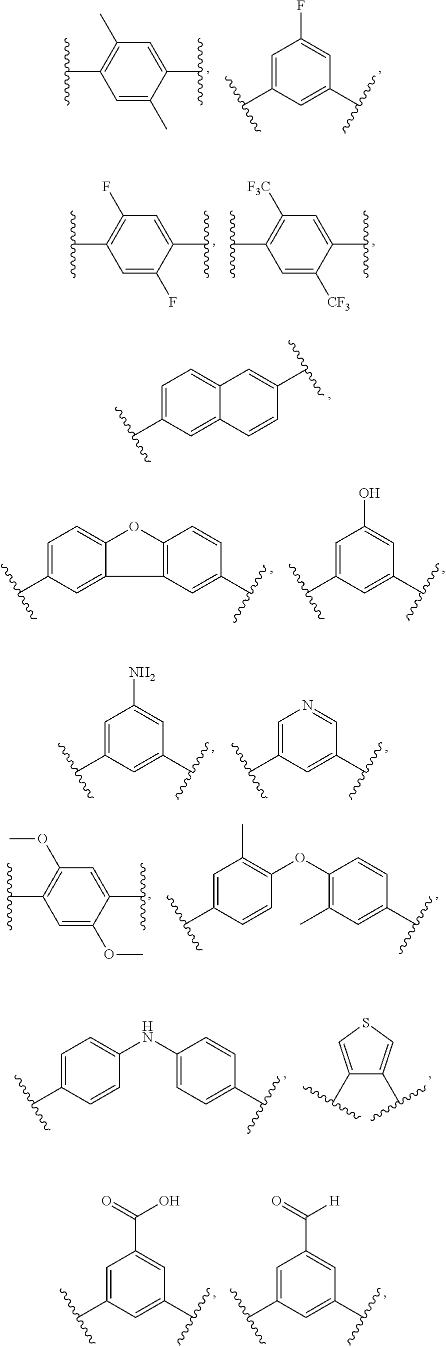

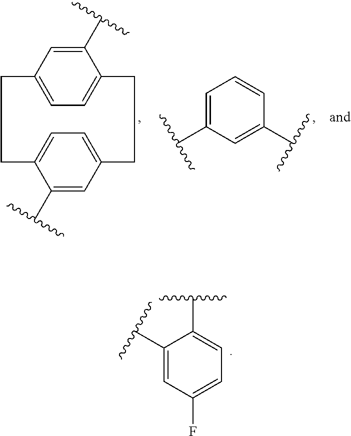

In certain embodiments, wherein A¹ and A² are independently a natural or unnatural amino acid, a plurality of natural or unnatural amino acids, a peptide, an oligopeptide, a polypeptide, or a protein.

In certain embodiments, A¹ comprises arginine, histidine, lysine, aspartic acid, glutamic acid, serine, threonine, asparagine, glutamine, proline, tyrosine, or tryptophan.

In certain embodiments, A² comprises arginine, histidine, lysine, aspartic acid, glutamic acid, serine, threonine, asparagine, glutamine, proline, tyrosine, or tryptophan.

In certain embodiments, A¹ and A² do not comprise cysteine or selenocysteine.

In certain embodiments, R¹ is H.

### Methods of Preparing Functionalization Complexes

In another aspect, provided herein is a method of preparing a palladium complex, wherein said method is represented by Scheme 4:

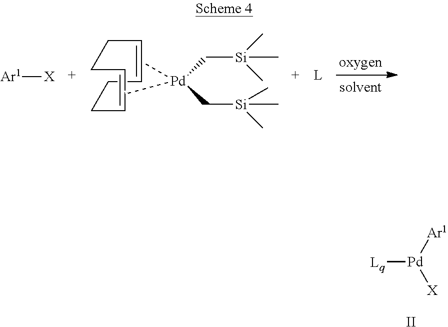

Ar¹ is optionally substituted aryl, heteroaryl, alkenyl, or cycloalkenyl;

X is a halide, inflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence represented by structure L:

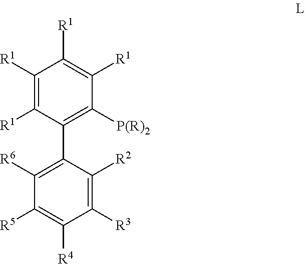

wherein

R is selected independently for each occurrence from the group consisting of alkyl, cycloalkyl, aryl, heteroaryl, aralkyl, heteroaralkyl, and —(CH₂)ₘ—R⁸⁰;

R¹ is selected independently for each occurrence from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R² is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R³ is selected from the group consisting of halogen, alkyl, cycloalkyl, heterocycloalkyl, aryl, heteroaryl, aralkyl, heteroaralkyl, —OR⁸, —N(R⁸)₂, Si(R⁸)₃, —R⁷, and —(CH₂)ₘ—R⁸⁰;

R⁴ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;

R⁵ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;

R⁶ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R⁷ is selected independently for each occurrence from the group consisting of —C(O)OM, —C(O)SM, —C(S)SM, —C(NR⁸)OM, —C(NR⁸)SM, —S(O)OM, —S(O)SM, —S(O)₂OM, —S(O)₂SM, —P(O)(OM)₂, —P(O)(OR⁸)OM, —P(O)(OR⁸)NR⁸M, —P(O)(OR⁸)SM, —N(R⁸)₃M, —P(R⁸)₃M, —P(OR⁸)₃M and —N(R⁸)C(NR⁸R⁸)NR⁸R⁸M;

R⁸ is selected independently for each occurrence from the group consisting of hydrogen, alkyl, cycloalkyl, heterocycloalkyl, aryl, heteroaryl, aralkyl, and heteroaralkyl;

M is an alkali metal or an alkali earth metal;

R⁸⁰ represents an unsubstituted or substituted aryl, a cycloalkyl, a cycloalkenyl, a heterocycle, or a poly cycle;

m is independently for each occurrence an integer in the range 0 to 8 inclusive;

provided that at least one of R³, R⁴ or R⁵ is R⁷;

the ligand is achiral or, when chiral, is a single stereoisomer or a mixture of stereoisomers;

q is 1 or 2; and

solvent is a polar protic solvent, a polar aprotic solvent, or a non-polar solvent;

wherein the method occurs in the presence of oxygen.

In another aspect, provided herein is a method of preparing a palladium complex, wherein said method is represented by Scheme 5:

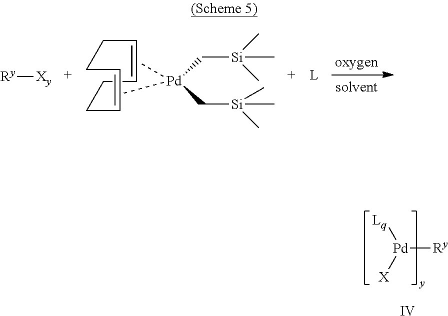

R^{y} is an optionally substituted bridging moiety, comprising an aromatic group, a heteroaromatic group, an alkene group, or a cycloalkene group;

y is 2, 3, 4, 5, or 6:

X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence represented by structure L:

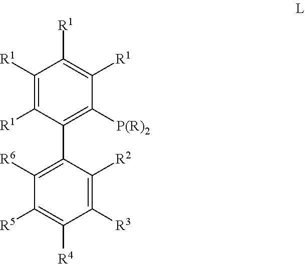

wherein

R is selected independently for each occurrence from the group consisting of alkyl, cycloalkyl, aryl, heteroaryl, aralkyl, heteroaralkyl, and —(CH₂)ₘ—R⁸⁰;

R¹ is selected independently for each occurrence from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R² is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R³ is selected from the group consisting of halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;

R⁴ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;

R⁵ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;

R⁶ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R⁷ is selected independently for each occurrence from the group consisting of —C(O)OM, —C(O)SM, —C(S)SM, —C(NR⁸)OM, —C(NR⁸)SM, —S(O)OM, —S(O)SM, —S(O)₂OM, —S(O)₂SM, —P(O)(OM)₂, —P(O)(OR⁸)OM, —P(O)(OR⁸)NR⁸M, —P(O)(OR⁸)SM, —N(R⁸)₃M, —P(R⁸)₃M, —P(OR⁸)₃M and —N(R⁸)C(NR⁸R⁸)NR⁸R⁸M;

R⁸ is selected independently for each occurrence from the group consisting of hydrogen, alkyl, cycloalkyl, heterocycloalkyl, aryl, heteroaryl, aralkyl, and heteroaralkyl;

M is an alkali metal or an alkali earth metal;

R⁸⁰ represents an unsubstituted or substituted aryl, a cycloalkyl, a cycloalkenyl, a heterocycle, or a poly cycle;

m is independently for each occurrence an integer in the range 0 to 8 inclusive; provided that at least one of R³, R⁴ or R⁵ is R⁷;

the ligand is achiral or, when chiral, is a single stereoisomer or a mixture of stereoisomers;

q is 1 or 2; and

solvent is a polar protic solvent, a polar aprotic solvent, or a non-polar solvent;

wherein the method occurs in the presence of oxygen.

In some embodiments of the compounds disclosed herein, q is an integer from 0-3. In certain embodiments, q is an integer from 1-3. In certain embodiments, q is 1 or 2. In more particular embodiments, q is 1. In certain embodiments in which q is 2 or 3, one instance of L is covalently connected via a linker moiety to one or more other instances of L. In such certain embodiments, M, taken together with two or three instances of ligand, is a cyclic or bicyclic structure.

In some embodiments of the compounds disclosed herein, the ligand L is a ligand described in U.S. Pat. No. 7,858,784, which is hereby incorporated by reference in its entirety.

In some embodiments of the compounds disclosed herein, the ligand L is a ligand described in U.S. Patent Application Publication No. 2011/0015401, which is hereby incorporated by reference in its entirety.

In some embodiments of the compounds disclosed herein, the ligand L is a ligand described in U.S. Pat. No. 7,560,596, which is hereby incorporated by reference in its entirety.

In some embodiments of the compounds disclosed herein, the ligand L is a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine. In certain embodiments, the ligand L is a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, or a triarylphosphonate. In certain embodiments, the ligand L is independently for each occurrence a triarylphosphine, a dialkylarylphosphine, or an (alkenyl)(alkyl)(aryl)phosphine. In some embodiments, the aryl is a substituted biphenyl.

In some embodiments of the compounds disclosed herein, the ligand L is selected from the group consisting of

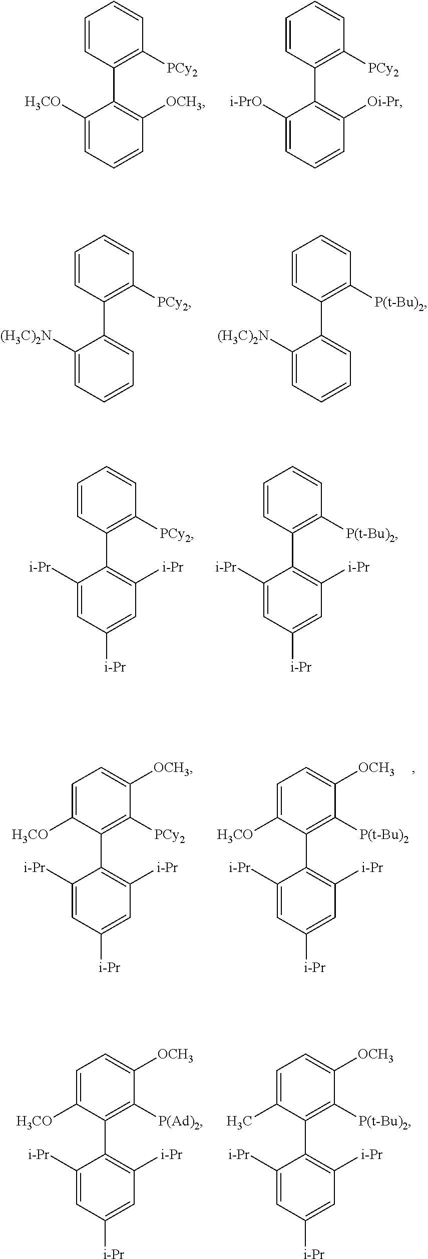

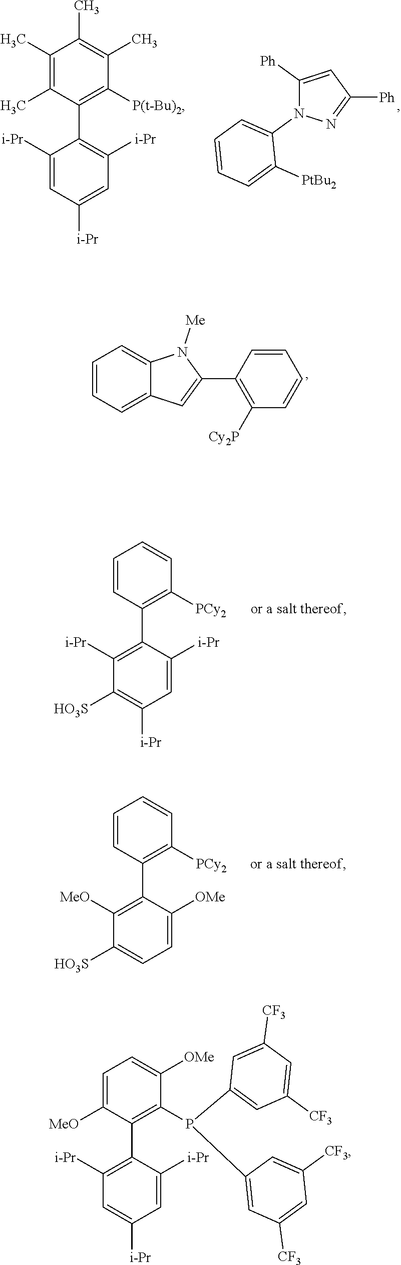

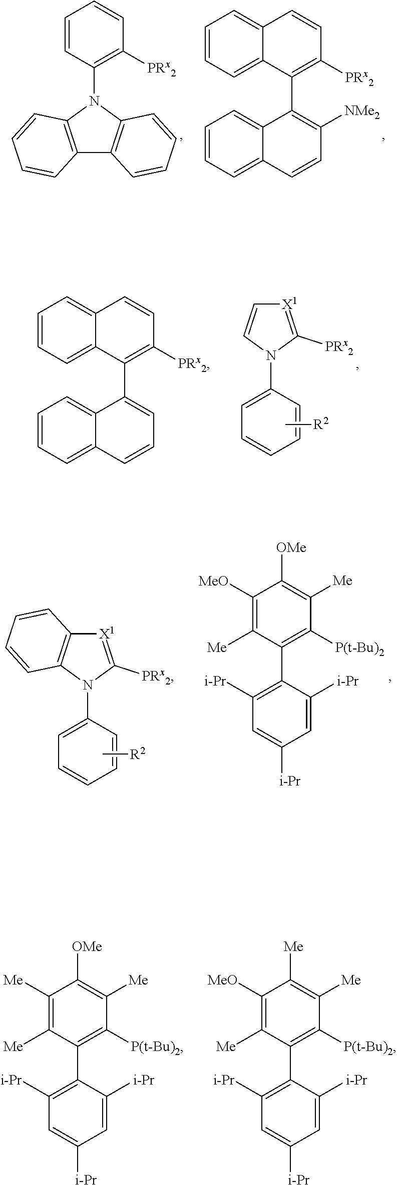

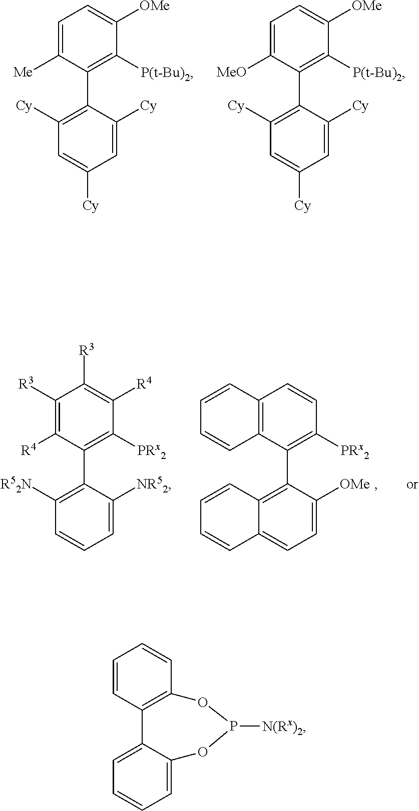

wherein

R^{x} is independently for each occurrence alkyl, aralkyl, cycloalkyl, or aryl;

X¹ is CH or N;

R² is H or alkyl;

R is H or alkyl;

R⁴ is H, alkoxy, or alkyl; and

R⁵ is alkyl or aryl.

In some embodiments of the compounds disclosed herein, the ligand L is

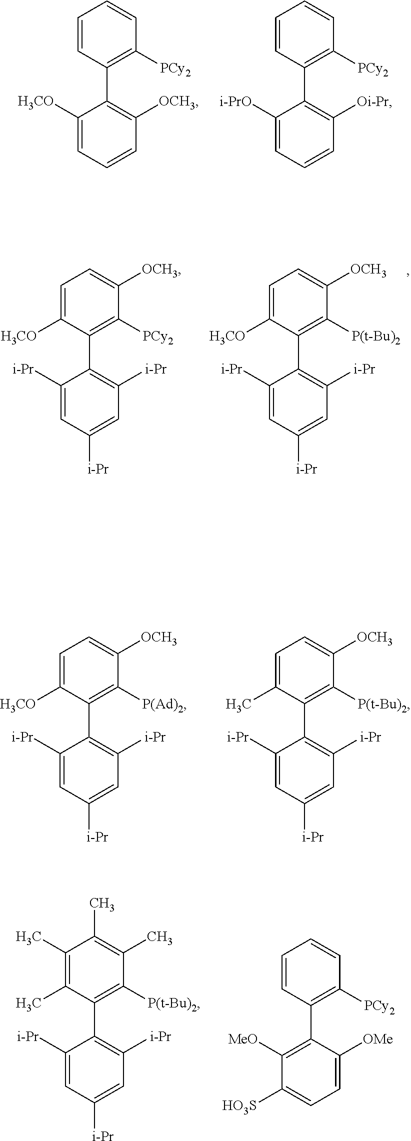

or a salt thereof,

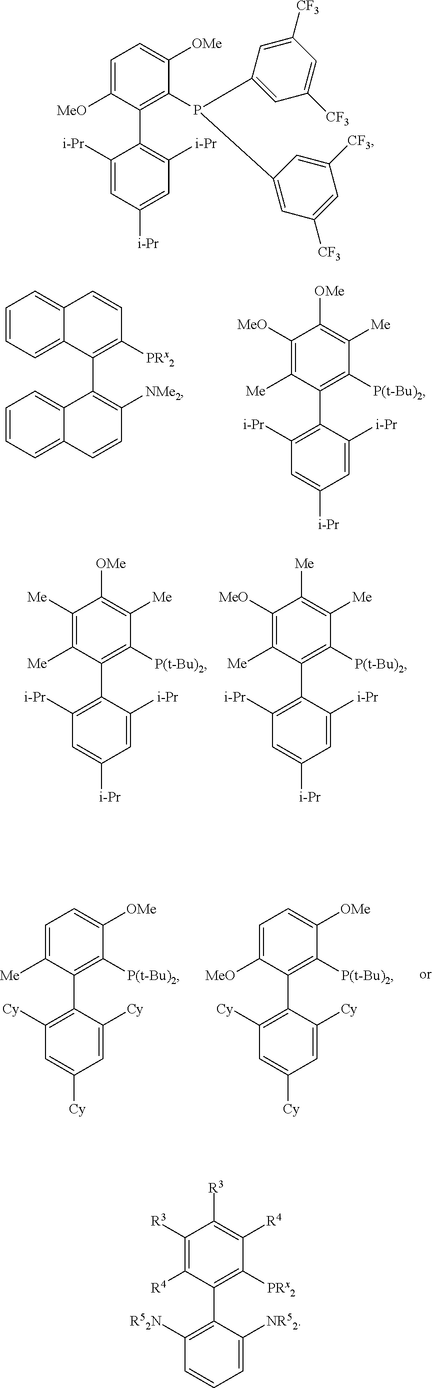

In some embodiments of the compounds disclosed herein, the ligand L is

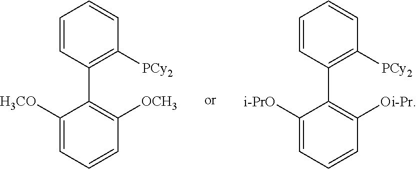

In some embodiments of the compounds disclosed herein, the ligand L is independently for each occurrence

represented by structure L:

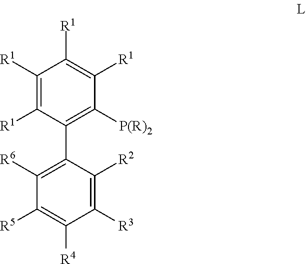

wherein

R is selected independently for each occurrence from the group consisting of alkyl, cycloalkyl, aryl, heteroaryl, aralkyl, heteroaralkyl, and —(CH₂)ₘ—R⁸⁰;

R¹ is selected independently for each occurrence from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R² is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R³ is selected from the group consisting of halogen, alkyl, cycloalkyl, heterocycloalkyl, and, heteroaryl, aralkyl, heteroaralkyl, —OR⁸, —N(R⁸)₂, —Si(R⁸)₃, —R⁷, and —(CH₂)ₘ—R⁸⁰;

R⁴ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;

R⁵ is selected from the group consisting of hydrogen, halogen, alkyl, cycloalkyl, heterocycloalkyl, and, heteroaryl, aralkyl, heteroaralkyl, —OR⁸, —N(R⁸)₂, —Si(R⁸)₃, R⁷, and —(CH₂)ₘ—R⁸⁰;

R⁶ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;

R⁷ is selected independently for each occurrence from the group consisting of —C(O)OM, —C(O)SM, —C(S)SM, —C(NR⁸)OM, —C(NR⁸)SM, —S(O)OM, —S(O)SM, —S(O)₂OM, —S(O)₂SM, —P(O)(OM)₂, —P(O)(OR⁸)OM, —P(O)(OR⁸)NR⁸M, —P(O)(OR⁸)SM, —N(R⁸)₃M, —P(R⁸)₃M, —P(C)R⁸)₃M and —N(R⁸)C(NR⁸R⁸)NR⁸R⁸M;

R⁸ is selected independently for each occurrence from the group consisting of hydrogen, alkyl, cycloalkyl, heterocycloalkyl, aryl, heteroaryl, aralkyl, and heteroaralkyl;

M is an alkali metal or an alkali earth metal;

R⁸⁰ represents an unsubstituted or substituted aryl, a cycloalkyl, a cycloalkenyl, a heterocycle, or a polycycle:

m is independently for each occurrence an integer in the range 0 to 8 inclusive;

provided that at least one of R³, R⁴ or R⁵ is R⁷; and

the ligand is achiral or, when chiral, is a single stereoisomer or a mixture of stereoisomers.

In some embodiments of the compounds disclosed herein, R is cyclohexyl; R¹ is hydrogen; and R² is alkyl or alkoxy.

In some embodiments of the compounds disclosed herein, R is cyclohexyl; R¹ is hydrogen; R² is alkyl or alkoxy; and R³ is R⁷.

In some embodiments of the compounds disclosed herein, R is cyclohexyl; R¹ is hydrogen; R³ is R⁷; and R⁴ and R⁵ are hydrogen.

In some embodiments of the compounds disclosed herein, R is cyclohexyl; R¹ is hydrogen; and R³ is R⁷.

In some embodiments of the compounds disclosed herein, R is cyclohexyl; R¹ is hydrogen; R² and R⁶ are selected independently from the group consisting of hydrogen, alkyl and alkoxy; R³ is R⁷; and R⁴ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰.

In some embodiments of the compounds disclosed herein, R is cyclohexyl; R¹ is hydrogen; R² and R⁶ are selected independently from the group consisting of hydrogen, alkyl and alkoxy; R³ is R⁷; and R⁴ and R² are hydrogen.

In some embodiments of the compounds disclosed herein, R is cyclohexyl; R¹ is hydrogen; R² and R⁶ are methoxy; R³ is —S(O)₂ONa; and R⁴ and R⁵ are hydrogen.

In some embodiments of the compounds disclosed herein, R is cyclohexyl; R¹ is hydrogen; R² and R⁶ are isopropyl; R⁴ is —S(O)₂ONa; and R³ and R⁵ are hydrogen.

In certain embodiments, the ligands of the present disclosure are represented by structure L and the attendant definitions, wherein R is selected independently for each occurrence from the group consisting of alkyl and cycloalkyl.

In certain embodiments, the ligands of the present disclosure are represented by structure L and the attendant definitions, wherein R is independently for each occurrence cycloalkyl.

In certain embodiments, the ligands of the present disclosure are represented by structure L and the attendant definitions, wherein R is cyclohexyl.

In certain embodiments, the ligands of the present disclosure are represented by structure L and the attendant definitions, wherein R¹ is hydrogen.

In certain embodiments, the ligands of the present disclosure are represented by structure L and the attendant definitions, wherein R² is alkyl or alkoxy.

In certain embodiments, the ligands of the present disclosure are represented by structure L and the attendant definitions, wherein R² is alkoxy.

In certain embodiments, the ligands of the present disclosure are represented by structure L and the attendant definitions, wherein R² is methoxy.

In certain embodiments, the ligands of the present disclosure are represented by structure L and the attendant definitions, wherein R² is alkyl.

In certain embodiments, the ligands of the present disclosure are represented by structure L and the attendant definitions, wherein R² is isopropyl.

In certain embodiments, the ligands of the present disclosure are represented by structure L and the attendant definitions, wherein R³ is R⁷; and R⁴ mid R³ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R³ is R⁷; and R⁴ and R³ are hydrogen.

In certain embodiments, the ligands of the present disclosure are represented by structure L and the attendant definitions, wherein R³ is —S(O)₂OM; and R⁴ and R³ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R^{x}K and —(CH₂)ₘ—R⁸⁰.

In certain embodiments, tire ligands of the present disclosure are represented by structure L and the attendant definitions, wherein R⁴ is R⁷; and R³ and R⁵ are selected independently from the group consisting of hydrogen, halogen, alkyl, cycloalkyl, heterocycloalkyl, aryl, heteroaryl, aralkyl, heteroaralkyl, —OR^{x}, —N(R⁸)₂, —Si(R⁸)₃, and —(CH₂)ₘ—R⁸⁰.

In certain embodiments, the ligands of the present disclosure are represented by structure L and the attendant definitions, wherein R⁴ is R⁷; and R³ mid R³ are hydrogen.

In certain embodiments, the ligands of the present disclosure are represented by structure L and the attendant definitions, wherein R⁴ is —S(O)₂OM; and R³ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰,

In certain embodiments, the ligands of the present disclosure are represented by structure L and the attendant definitions, wherein R is cyclohexyl; and R¹ is hydrogen.

In certain embodiments, the ligands of the present disclosure are represented by structure L and the attendant definitions, wherein R is cyclohexyl; R¹ is hydrogen; and R² is alkyl or alkoxy.

In certain embodiments, the ligands of the present disclosure are represented by structure L and the attendant definitions, wherein R is cyclohexyl; R¹ is hydrogen; and R² is alkoxy.

In certain embodiments, the ligands of the present disclosure are represented by structure L and the attendant definitions, wherein R is cyclohexyl; R¹ is hydrogen; and R² is methoxy.

In certain embodiments, the ligands of the present disclosure are represented by structure L and the attendant definitions, wherein R is cyclohexyl; R¹ is hydrogen; and R² is alkyl.

In certain embodiments, the ligands of the present disclosure are represented by structure L and the attendant definitions, wherein R is cyclohexyl; R¹ is hydrogen; and R² is isopropyl.

In certain embodiments, the ligands of the present disclosure are represented by structure L and the attendant definitions, wherein R is cyclohexyl; R¹ is hydrogen; R³ is R⁷; and R⁴ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and (CH₂)ₘ—R⁸⁰.

In certain embodiments, the ligands of the present disclosure are represented by structure L and the attendant definitions, wherein R is cyclohexyl; R¹ is hydrogen; R³ is R⁷; and R⁴ and R⁵ are hydrogen.

In certain embodiments, the ligands of the present disclosure are represented by structure L and the attendant definitions, wherein R is cyclohexyl; R¹ is hydrogen; R³ is —S(O)₂OM; and R⁴ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; R⁴ is R⁷; and R³ and R⁵ are selected independently from, the group consisting of hydrogen, halogen, alkyl, cycloalkyl, heterocycloalkyl, and, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; R⁴ is R⁷; and R³ and R⁵ are hydrogen.

In certain embodiments, the ligands of the present disclosure are represented by structure L and the attendant definitions, wherein R is cyclohexyl; R¹ is hydrogen; R⁴ is —S(O)₂OM; and R³ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; and R² and R⁶ are selected independently from the group consisting of hydrogen, alkyl and alkoxy.

In certain embodiments, the ligands of the present disclosure are represented by structure L and the attendant definitions, wherein R is cyclohexyl; R¹ is hydrogen; R² and R⁶ are selected independently from the group consisting of hydrogen, alkyl and alkoxy; R³ is R⁷; and R⁴ and R³ are selected independently from the group consisting of hydrogen, halogen, alkyl, cycloalkyl, heterocycloalkyl, and, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; R² and R⁶ are selected independently from the group consisting of hydrogen, alkyl and alkoxy; R³ is R⁷; and R⁴ and R³ are hydrogen.

In certain embodiments, the ligands of the present disclosure are represented by structure L and the attendant definitions, wherein R is cyclohexyl; R¹ is hydrogen; R² and R⁶ are selected independently from the group consisting of hydrogen, alkyl and alkoxy; R⁴ is R⁷; and R³ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and (CH₂)ₘ—R⁸⁰.

In certain embodiments, the ligands of the present disclosure are represented by structure L and the attendant definitions, wherein R is cyclohexyl; R¹ is hydrogen; R² and R⁶ are selected independently from the group consisting of hydrogen, alkyl and alkoxy; R⁴ is R⁷; and R³ and R⁵ are hydrogen.

In certain embodiments, the ligands of the present disclosure are represented by structure L and the attendant definitions, wherein R is cyclohexyl; R¹ is hydrogen; R² and R⁶ are alkoxy; R³ is —S(O)₂OM; and R⁴ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; R² and R⁶ are alkyl; R³ is —S(O)₂OM; and R⁴ and R⁵ are selected independently from the group consisting of hydrogen, halogen, alkyl, cycloalkyl, heterocycloalkyl, and, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; R² and R⁶ are alkoxy; R⁴ is —S(O)₂OM; and R³ and R⁵ are selected independently from the group consisting of hydrogen, halogen, alkyl, cycloalkyl, heterocycloalkyl, and, heteroaryl, aralkyl, heteroaralkyl, —OR⁸, —N(R⁸)₂, —Si(R⁸)₃, and —(CH₂)ₘ—R⁸⁰.

In certain embodiments, the ligands of the present disclosure are represented by structure L and the attendant definitions, wherein R is cyclohexyl; R¹ is hydrogen; R² and R⁶ are alkyl; R³ is —S(O)₂OM; and R⁴ and R⁵ are selected independently from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and (CH₂)ₘ—R⁸⁰.

In certain embodiments, the ligands of the present disclosure are represented by structure L and the attendant definitions, wherein R is cyclohexyl; R¹ is hydrogen; R² and R⁶ are methoxy; R³ is —S(O)₂ONa; and R⁴ and R⁵ are hydrogen.

In certain embodiments, the ligands of the present disclosure are represented by structure L and the attendant definitions, wherein R is cyclohexyl; R¹ is hydrogen; R² and R⁶ are methoxy; R³ and R⁵ are —S(O)₂ONa; and R⁴ is hydrogen.

In certain embodiments, the ligands of the present disclosure are represented by structure L and the attendant definitions, wherein R is cyclohexyl; R¹ is hydrogen; R² and R⁶ are isopropyl; R⁴ is —S(O)₂ONa; and R³ and R⁵ are hydrogen.

In some embodiments of the compounds disclosed herein, X is a halide (e.g., fluoride, chloride, bromide, iodide) or a triflate.

In some embodiments of the compounds disclosed herein, X is selected from the group consisting of boron tetrafluoride, tetraarylborates (such as B(C₆F₅)₄⁻ and (B[3,5-(CF₃)₂C₆H₃]₄)⁻), hexafluoroantimonate, phosphorus tetrafluoride, phosphorus hexafluoride, alkylsulfonate, haloalkylsulfonate, arylsulfonate, perchlorate, bis(alkylsulfonyl)amide, halide, bis(fluoroalkylsulfonyl)amide, bis(arylsulfonyl)amide, (fluoroalkylsulfonyl)-(fluoroalkyl-carbonyl)amide, nitrate, nitrite, sulfate, hydrogen sulfate, alkyl sulfate, aryl sulfate, carbonate, bicarbonate, carboxylate, phosphate, hydrogen phosphate, dihydrogen phosphate, phosphinate, and hypochlorite.

In some embodiments of the compounds disclosed herein, X is alkylsulfonate; and the alkyl is substituted alkyl. In certain embodiments, X is alkylsulfonate; and tire alkyl is unsubstituted alkyl.

In some embodiments of the compounds disclosed herein, X is alkylsulfonate; and the alkyl is methyl, ethyl, propyl, or butyl. In certain embodiments, X is alkylsulfonate; and the alkyl is methyl or ethyl.

In some embodiments of the compounds disclosed herein, X is haloalkylsulfonate. In certain embodiments, X is fluoroalkylsulfonate.

In some embodiments of the compounds disclosed herein, X is fluoromethylsulfonate. In certain embodiments, X is trifluoromethylsulfonate.

In some embodiments of the compounds disclosed herein, X is cycloalkylalkylsulfonate. In certain embodiments, X is

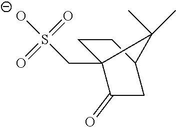

or its enantiomer.

In some embodiments of the compounds disclosed herein, q is 1 or 2. In certain embodiments, q is 1.

In certain embodiments of the compounds described herein, Ar¹ is optionally substituted aryl, heteroaryl, alkenyl, or cycloalkenyl. In certain embodiments, Ar¹ is as described above.

In some embodiments, the methods of preparing a palladium complex require an oxygen-free environment. In some embodiment, the preparation is in a nitrogen-filled glovebox.

In some embodiments, the method of preparing a palladium complex occurs in the presence of oxygen. In some embodiments, the method is at room temperature. In some embodiments, the methods form air-stable complexes.

In some embodiments, the solvent is an ether. In some embodiments, the solvent is tetrahydrofuran. In some embodiments, the solvent is as described above.

### Exemplary Polymetalated Reagents

In certain embodiments, the disclosure relates to a compound of formula IV:

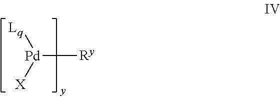

wherein, independently for each occurrence,

R^{y} is an optionally substituted bridging moiety, comprising an aromatic group, a heteroaromatic group, an alkene group, or a cycloalkene group;

y is 2, 3, 4, 5, or 6;

X is a halide, Inflate, tetrafluoroborate, tetraarylborate, hexafluoroantimonate, bis(alkyl sulfonyl)amide, tetrafluorophosphate, hexafluorophosphate, alkyl sulfonate, haloalkylsulfonate, and sulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;

L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine; and

q is 1 or 2.

In certain embodiments, the disclosure relates to any one of the compounds described herein, wherein R^{y} is an optionally substituted bifunctional bridging moiety or an optionally substituted trifunctional bridging moiety.

In certain embodiments, the disclosure relates to any one of the compounds described herein, wherein R^{y} comprises an aromatic group.

In certain embodiments, the disclosure relates to any one of the compounds described herein, wherein R^{y} is optionally substituted

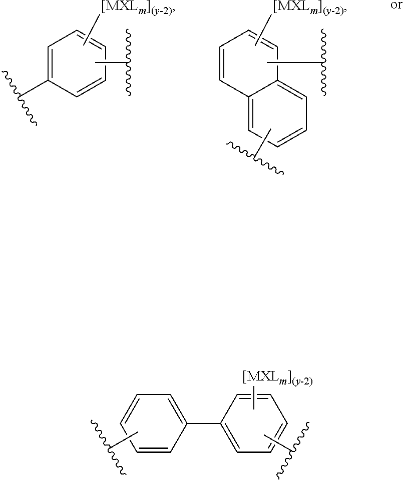

In certain embodiments, the disclosure relates to any one of the compounds described herein, wherein y is 2; and R^{y} is selected from, the group consisting of

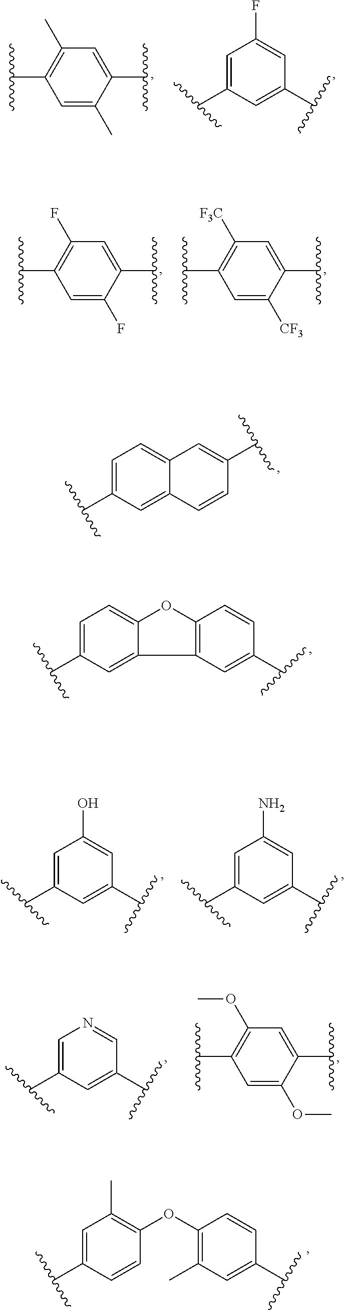

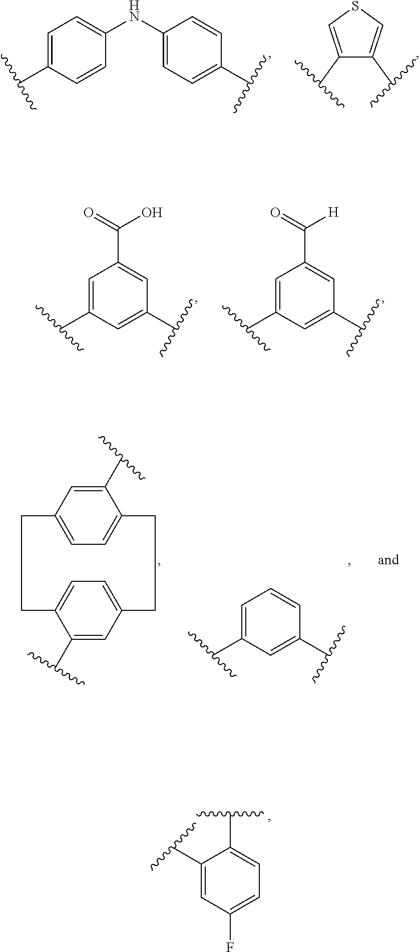

wherein any of the bifunctional bridging moieties may be optionally substituted.

In certain embodiments, the disclosure relates to any one of the compounds described herein, wherein y is 3; and R^{y} is selected from the group consisting of

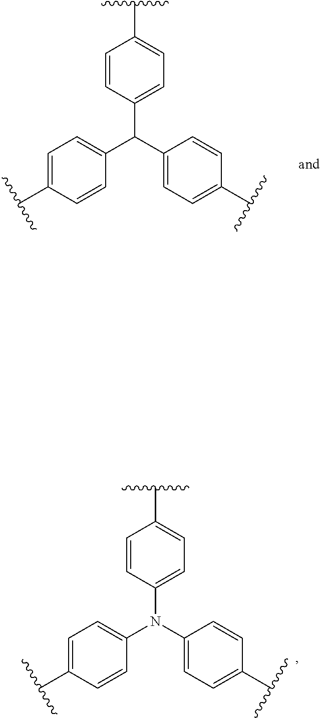 [SMILES: *c1ccc(C(c2ccc(*)cc2)c2ccc(*)cc2)cc1 ; *c1ccc(N(c2ccc(*)cc2)c2ccc(*)cc2)cc1]

wherein any of the trifunctional bridging moieties may be optionally substituted.

### Exemplary Conjugated Compounds

In certain embodiments, the disclosure relates to a hybrid composition, wherein the hybrid composition comprises a tinker, a compound of substructure III, and a detectable moiety; and the linker links the compound to the detectable moiety.

In certain embodiments, the disclosure relates to any one of the aforementioned hybrid compositions, wherein the detectable moiety is a fluorescent moiety, a dye moiety, a radionuclide, a drag molecule, an epitope, or an MRI contrast agent.

In certain embodiments, the disclosure relates to a hybrid composition, wherein the hybrid composition comprises a linker, a compound of substructure III, and a biomolecule; and the linker links the compound to the biomolecule.

In certain embodiments, the disclosure relates to any one of the aforementioned hybrid compositions, wherein the biomolecule is a protein.

In certain embodiments, the disclosure relates to any one of the aforementioned hybrid compositions, wherein the protein is an antibody. In some embodiments, the antibody is trastuzumab.

In certain embodiments, the disclosure relates to any one of the aforementioned hybrid compositions, wherein tire biomolecule is DNA, RNA, or peptide nucleic acid (PNA).

In certain embodiments, the disclosure relates to any one of the aforementioned hybrid compositions, wherein the biomolecule is siRNA.

In certain embodiments, the disclosure relates to a hybrid composition, wherein the hybrid composition comprises a linker, a compound of substructure III, and a polymer; and the linker links the compound to the polymer.

In certain embodiments, the disclosure relates to any one of the aforementioned hybrid compositions, wherein the polymer is polyethylene glycol.

### Exemplary Peptides, Oligopeptides, Polypeptides, and Proteins

In certain embodiments, the disclosure relates to a method to generate a peptide, an oligopeptide, a polypeptide, or a protein, wherein the peptide, oligopeptide, polypeptide, or protein comprises substructure III.

In certain embodiments, the disclosure relates to a peptide, an oligopeptide, a polypeptide, or a protein, wherein the peptide, oligopeptide, polypeptide, or protein comprises a plurality of substructures comprising substructure III.

In certain embodiments, the disclosure relates to any one of the peptides, oligopeptides, polypeptides, or proteins described herein.

In certain embodiments, the disclosure relates to a method to generate a peptide, an oligopeptide, a polypeptide, or a protein, wherein the peptide, oligopeptide, polypeptide, or protein comprises substructure V.

In certain embodiments, the disclosure relates to a peptide, an oligopeptide, a polypeptide, or a protein, wherein the peptide, oligopeptide, polypeptide, or protein comprises a plurality of substructures comprising substructure V.

In certain embodiments, the disclosure relates to a method to generate a peptide, an oligopeptide, a polypeptide, or a protein, wherein the peptide, oligopeptide, polypeptide, or protein comprises substructure VI.

In certain embodiments, the disclosure relates to a peptide, an oligopeptide, a polypeptide, or a protein, wherein the peptide, oligopeptide, polypeptide, or protein comprises a plurality of substructures comprising substructure VI,

In certain embodiments, the disclosure relates to a peptide, an oligopeptide, a polypeptide, or a protein, or a method involving the peptide, oligopeptides, polypeptide, or protein, described in US published patent application publication number US 2014/0113871, which is hereby incorporated by reference in its entirety.

### Exemplary Therapeutic Methods

Anti body-drag conjugates (ADCs) are an emerging class of anti-cancer therapeutics. Highly cytotoxic small molecule drugs are conjugated to antibodies to create a single molecular entity, ADCs combine the high efficacy of small molecules with the target specificity of antibodies to enable the selective delivery of drug payloads to cancerous tissues, which, reduces the systematic toxicity of conventional small molecule drags.

Traditionally, ADCs are prepared by conjugating small molecule drugs to either cysteines generated from reducing an internal disulfide bond or surface-exposed lysines. Because multiple lysines and cysteines are present in antibodies, these conventional approaches usually lead to heterogeneous products with undefined drug-antibody ratio, which might cause difficulty for manufacturing and characterization. Furthermore, each individual antibody-drug conjugate may exhibit different pharmacokinetics, efficacy, and safety profiles, hindering a rational approach to optimizing ADC-based cancer treatment.

Recent studies showed that ADCs prepared using site-specific conjugation techniques exhibited improved pharmacological profiles.

So, in certain embodiments, the disclosure relates to an ADC with defined position of drag-attachment and defined drag to antibody ratio. In certain embodiments, the ADCs of the disclosure permit rational optimization of ADC-based therapies. In certain embodiments, the ADC comprises a structure of any one of the compounds generated by the methods described herein. In certain embodiments, the drug-to-antibody ratio is about 2:1, about 3:1, about 4:1, about 5:1, about 6:1, about 7:1, about 8:1, about 9:1, about 10:1, about 11:1, or about 12:1.

In certain embodiments, the disclosure relates to any one of the ADCs mentioned herein, comprising a therapeutic selected from the group consisting of trametinib, topotecan, abiraterone, dabrafenib, vandetanib, camptothecin, SN-38, monomethyl auristatin E (MMAE), duocarmycin SA, indibulin, tubulysin A, and maytansine covalently conjugated to an antibody. In some embodiments, the antibody targets a cell surface receptor that is over-expressed in a cancer cell.

In certain embodiments, the disclosure relates to any one of the ADCs mentioned herein, comprising monomethyl auristatin E (MMAE) covalently conjugated to an antibody, wherein the antibody targets a cell surface receptor that is over-expressed in a cancer cell. MMAE is a highly toxic antimitotic agent that inhibits cell division by blocking tubulin polymerization, MMAE has been successfully conjugated to antibodies targeting human CD30 to create ADCs that have been approved by FDA to treat Hodgkin lymphoma as well as anaplastic large-cell lymphoma. In certain embodiments, the disclosure relates to a method for the selective synthesis of an ADC comprising MMAE covalently conjugated to an antibody. In some embodiments, the antibody is trastuzumab.

In certain embodiments, the disclosure relates to any one of the ADCs mentioned herein, wherein the antibody targets cell receptors CD30, CD22, CD33, human epidermal growth factor receptor 2 (HER2), or epidermal growth factor receptor (EGFR). It should be noted that by conjugating drags to antibodies targeting different receptors, the ADCs prepared should be useful for treating different cancers.

### Definitions

For convenience, before further description of the present disclosure, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term, “heteroatom” is art-recognized and refers to an atom of any element other than carbon or hydrogen. Illustrative heteroatoms include boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths but with at least two carbon atoms. Preferred alkyl groups are lower alkyls. In preferred embodiments, a substituent designated herein as alkyl is a lower alkyl.

The term “aralkyl”, as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyl oxy.

The term “alkoxycarbonyl” means an alkoxy group, as defined herein, appended to the parent molecular moiety through a carbonyl group, represented by —C(═O), as defined herein. Representative examples of alkoxycarbonyl include, but are not limited to, methoxycarbonyl, ethoxycarbonyl, and tert-butoxycarbonyl.

The term “carboxy” as used herein, means a —CO₂H group.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio. The terms “arylthio,” “alkenylthio” and “arylakylthio,” for example, are likewise defined.

The term “am ide” as used herein, means —NHC(═O), wherein the amido group is bound to the parent molecular moiety through the nitrogen. Examples of amido include alkylamido such as CH₃C(═O)N(H)— and CR₃CH₂C(═O)N(H)—.

The term “aryl” as used herein includes 5-, 6- and 7-membered aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or fire like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The abbreviations Me, Et, Ph, Tf, Nf, Ts, Ms, and dba represent methyl, ethyl, phenyl, trifluoromethanesulfonyl, nonafluorobutanesulfonyl, p-toluenesulfonyl, methanesulfonyl, and dibenzylideneacetone, respectively. Also, “DCM” stands for dichloromethane; “rt” stands for room temperature, and may mean about 20° C., about 21° C., about 22° C., about 23° C., about 24° C., about 25° C., or about 26° C.; “THF” stands for tetrahydrofuran; “BINAP” stands for 2,2′-bis(diphenylphosphino)-1,1′-binaphthyl; “dppf” stands for 1,1′-bis(diphenylphosphino)ferrocene; “dppb” stands for 1,4-bis(diphenylphosphinobutane; “dppp” stands for 1,3-bis(diphenylphosphino)propane; “dppe” stands for L2-bis(diphenylphosphino)ethane. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in tire art are hereby incorporated by reference.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “non-coordinating anion” relates to a negatively charged moiety that interacts weakly with cations. Non-coordinating anions are useful in studying tire reactivity of electrophilic cations, and are commonly found as counterions for cationic metal complexes with an unsaturated coordination sphere. In many cases, non-coordinating anions have a negative charge that is distributed symmetrically over a number of electronegative atoms. Salts of these anions are often soluble non-polar organic solvents, such as dichloromethane, toluene, or alkanes.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, CN, or the like.

The term “heteroatom” as used herein means an atom of any element other than carbon or hydrogen. Preferred heteroatoms are nitrogen, oxygen, sulfur and phosphorous.

As used herein, the term “nitro” means —NO₂; the term “halogen” or “halo” designates —F, —Cl, —Br or —I; the term “sulfhydryl” means —SH; the term “hydroxyl” means —OH; the term “sulfonyl” means —SO₂—; and the term, “cyano” as used herein, means a —CN group.

The term “haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The terms “amine” and “amino” are art recognized and refer to both unsubstituted and substituted amines, e.g., a moiety that can be represented by the general formula:

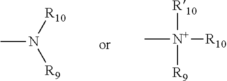

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In preferred embodiments, only one of R₉ or R₁₀ can be a carbonyl, e.g., R₉, R₁₀ and the nitrogen together do not form an imide. In even more preferred embodiments, R₉ and R₁₀ (and optionally R′₁₀) each independently represent a hydrogen, an alkyl, an alkenyl, or (CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The definition of each expression, e.g., alkyl, m, n, and the like, when it occurs more than once in any structure, is intended to be independent of its definition elsewhere in the same structure.

The terms triflyl (Tf), tosyl (-Ts), mesyl (-Ms), and nonaflyl are art-recognized and refer to trifluoromethanesulfonyl, p-toluenesulfonyl, methanesulfonyl, and nonafluorobutanesulfonyl groups, respectively. The terms triflate (-OTf), tosylate (-OTs), mesylate (-OMs), and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The phrase “protecting group” as used herein means temporary modifications of a potentially reactive functional group which protect it from undesired chemical transformations. Examples of such protecting groups include silyl ethers of alcohols, and acetals and ketals of aldehydes and ketones, respectively. In embodiments of the disclosure, a carboxylate protecting group masks a carboxylic acid as an ester. In certain other embodiments, an amide is protected by an amide protecting group, masking the —NH₂ of the amide as, for example, —NH(alkyl), or —N(alkyl)₂. The field of protecting group chemistry has been reviewed (Greene, T. W.; Writs, P. G. M. Protective Groups in Organic Synthesis, 2^{nd} ed.; Wiley: New York, 1991).

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described hereinabove. The permissible substituents can be one or more and the same or different for appropriate organic compounds. For purposes of this disclosure, the heteroatoms, such as nitrogen, may have hydrogen substituents and/or any permissible substituents of organic compounds described herein which satisfy the valencies of the heteroatoms,

A “polar protic solvent” as used herein is a solvent having a dipole moment of about 1.4 to 4.0 D, and comprising a chemical moiety that participates in hydrogen bonding, such as an O—H bond or an N—H bond. Exemplary polar protic solvents include methanol, ethanol, n-propanol, isopropanol, n-butanol, isobutanol, ammonia, water, and acetic acid.

A “polar aprotic solvent” as used herein means a solvent having a dipole moment of about 1.4 to 4.0 D that lacks a hydrogen bonding group such as O—H or N—H. Exemplary polar aprotic solvents include acetone, N,N-dimethylformamide, acetonitrile, ethyl acetate, dichloromethane, tetrahydrofuran, and dimethylsulfoxide.

A “non-polar solvent” as used herein means a solvent having a low dielectric constant (<5) and low dipole moment of about 0.0 to about 1.2. Exemplary nonpolar solvents include pentane, hexane, cyclohexane, benzene, toluene, chloroform, and diethyl ether.

For purposes of this disclosure, the chemical elements are identified in accordance with the Periodic Table of the Elements, CAS version, Handbook of Chemistry and Physics, 67th Ed., 1986-87, inside cover,

### EXEMPLIFICATION

The disclosure may be understood with reference to the following examples, which are presented for illustrative purposes only and which are non-limiting. The substrates utilized in these examples were either commercially available, or were prepared from commercially available reagents.

### General Reagent Information

1-[Bis(dimethylamino)methylene]-1H-1,2,3-triazolo[4,5-b]pyridinium 3-oxide hexafluorophosphate (HATU), D-Biotin, Fmoc-Rink amide linker, Fmoc-L-Gly-OH, Fmoc-L-Leu-OH, Fmoc-L-Lys(Boc)-OH, Fmoc-L-Ala-OH, Fmoc-L-Cys(Trt)-OH, Fmoc-L-Gln(Trt)-OH, Fmoc-L-Asn(Trt)-OH, Fmoc-L-Glu(OtBu)-OH, Fmoc-L-Arg(Pbf)-GH, Fmoc-L-Phe-OH, Fmoc-L-Ser(tBu)-OH, Fmoc-L-Thr(tBu)OH, Fmoc-L-Tyr(tBu)-OH, and Fmoc-L-His(Trt)-OH were purchased from Chem-Impex International (Wood Dale, Ill.). Peptide synthesis-grade N,N-dimethylformamide (DMF), dichloromethane (CH₂Cl₂), diethyl ether, HPLC-grade acetonitrile, and guanidine hydrochloride were obtained from VWR International (Philadelphia, Pa.). Aryl halides and aryl trifluoromethanesulfonates were purchased from Aldrich Chemical Co., Alfa Aesar, or Matrix Scientific and were used without additional purification. All deuterated solvents were purchased from Cambridge Isotopes and used without further purification. All other reagents were purchased from Sigma-Aldrich and used as received.

All reactions with peptides were set up on the bench top and carried out under ambient conditions. Anhydrous tetrahydrofuran, pentane, cyclohexane, and acetonitrile were purchased from Aldrich Chemical Company in SURESEAL® bottles and were purged with argon before use.

### General Analytical Information

All small-molecule organic and organometallic compounds were characterized by ¹H. ¹³C NMR, and IR spectroscopy, as well as elemental analysis or high-resolution mass spectrometry (unless otherwise noted). ¹⁹F NMR spectroscopy was used for organometallic complexes containing fluorine atoms. ³¹P NMR spectroscopy was used for characterization of palladium complexes. Copies of the ¹H, ¹³C, ³¹P, and ¹⁹F NMR spectra can be found at the end of the Supporting Information. Nuclear Magnetic Resonance spectra were recorded on a Bruker 400 MHz instrument and a Varian 300 MHz instrument. Unless otherwise stated, all ¹H NMR experiments are reported in δ units, parts per million (ppm), and were measured relative to the signals of the residual proton resonances CH₂Cl₂ (5.32 ppm) in the deuterated solvents. All ¹³C NMR spectra are measured decoupled from ¹H nuclei and are reported in δ units (ppm) relative to CD₂Cl₂ (54.00 ppm), unless otherwise stated. All ³¹P NMR spectra are measured decoupled from ³H nuclei and are reported relative to H₃PO₄ (0.00 ppm). ¹⁹F NMR spectra are measured decoupled from ¹H nuclei and are reported in ppm relative to CFCl₃ (0.00 ppm) or α,α,α-trifluorotoluene (−63.72 ppm). All FT-IR spectra were recorded on a Thermo Scientific—Nicolet iS5 spectrometer (iD5 ATR—diamond). Elemental analyses were performed by Atlantic Microlabs Inc., Norcross, Ga.

### LC-MS Analysis

LC-MS chromatograms and associated mass spectra were acquired using Agilent 6520 ESI-Q-TOF mass spectrometer. Solvent compositions used in tire LC-MS are 0.1% formic acid in H₂O (solvent A) and 0.1% formic acid in acetonitrile (solvent B). The following LC-MS method was used:

Method A LC conditions: Zorbax 300SB C3 column: 2.1×150 mm, 5 μm, column temperature: 40° C., gradient: 0-3 min 5% B, 3-8 min 5-95% B, 8-9 min 95% B, flow rate: 0.8 mL/min. MS conditions: positive electrospray ionization (ESI) extended dynamic mode in mass range 300-3000 m/z, temperature of drying gas=350° C., flow rate of drying gas=11 L/min, pressure of nebulizer gas=60 psi, the capillary, fragmentor, and octapole rf voltages were set at 4000, 175, and 750, respectively.

### Determination of Bioconjugation and Macrocyclization Yields

Data were processed using Agilent MassHunter software package. All reported yields were determined by integrating total ion current (TIC) spectra. First, the peak areas for all relevant peptide-containing species on the chromatogram were integrated using Agilent MassHunter software package. Since no peptide-based side products were generated in the experiments, the yields shown in Table 2 were determined as follows: % yield=Sₚᵣ/Sₜₒₜₐₗ where Sₚᵣ is tire peak area of the product and Sₜₒₜₐₗ is the peak area of combined peptide-containing species (product and starting material). For protein bioconjugation, deconvolved masses of proteins were obtained using maximum entropy algorithm. LC-MS data shown were acquired using Method A, unless otherwise noted. Mass spectrum insets correspond to the integration of the TIC peak unless otherwise noted.

### Example 1—Synthesis of Palladium Reagents

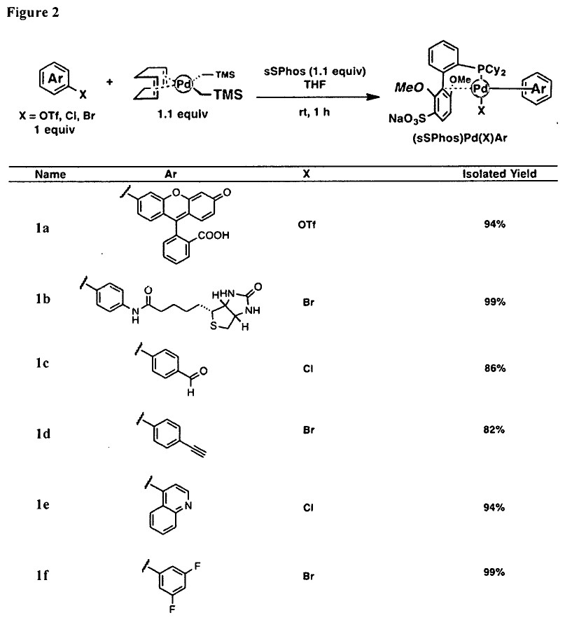

Several sSPhos-ligated palladium reagents were prepared on tire bench top, without tire use of a glovebox or advanced oxygen-free synthetic techniques. It was found that a mixture of aryl halide (ArX), ligand, and [(1,5-COD)Pd(CH₂TMS)₂] dissolved in tetrahydrofuran (THF) afforded the desired reagents in excellent yields after an hour of stirring (FIG. 2 and below) and subsequent filtration. These easily purified palladium reagents, sSPhosPd(X)Ar, are air-stable and easily storable under ambient atmosphere and temperature for upwards of 12 months without displaying noticeably diminished reactivity.

In order to showcase the utility of this methodology for chemical biologists, several types of bench-stable palladium reagents were isolated using this procedure. A fluorescein dye (1a), biotin labeling reagent (1b), bioconjugate handles for further diversification (1c and 1d), heterocycles (1e), and fluorinated aromatic compounds (1f) were prepared in good to excellent yield.

### General Procedure for the Synthesis of Mono-Palladium Oxidative Addition

### Complexes.

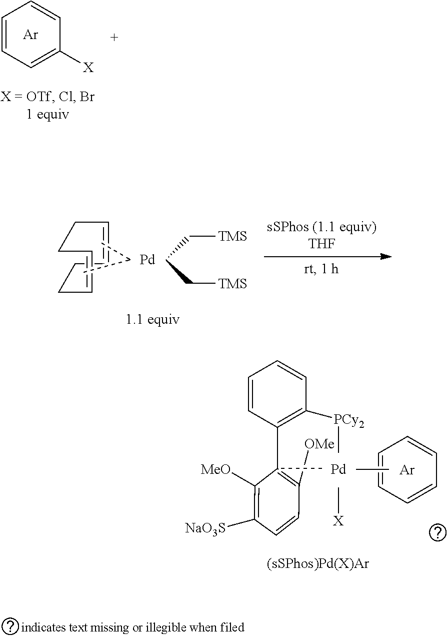

Palladium reagents were synthesized following the scheme of FIG. 2. A scintillation vial (10 mL), equipped with a magnetic stir bar, was charged with RuPhos (1.1 equiv) or sSPhos (1.1 equiv), Ar—X (1 equiv), and tetrahydrofuran. Solid [(1,5-COD)Pd(CH₂SiMe₃)₂] (Vinogradova, E. V., et al, Organometallic palladium reagents for cysteine bioconjugation. Nature, 526, 687-691 (2015)) (1.1 equiv) was added rapidly in one portion and the resulting solution was stirred for 1 h at rt. After this time, pentane (3 mL) was added and the resulting mixture was placed into a −20° C. freezer for 2 h. The vial was removed from the freezer and, in the air, the resulting precipitate was filtered, washed with pentane (5×3 mL), and dried under reduced pressure to afford the oxidative addition complex.

### Exemplary Oxidative Addition Complexes

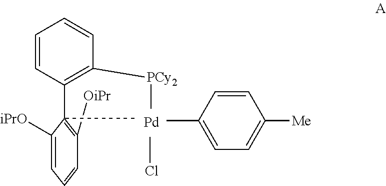

Following the general procedure, a mixture containing 4-chlorotoluene (6.4 μL, 0.054 mmol), RuPhos (28 mg, 0.06 mmol), and [(1,5-COD)Pd(CH₂SiMe₃)₂] (25 mg, 0.06 mmol) was stirred at rt in a nitrogen-filled glovebox in cyclohexane (1.5 mL) for 18 h. General work-up afforded A as a grey solid (37 mg, 96%).

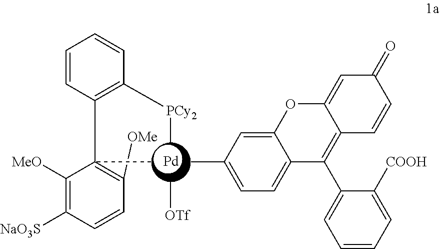

Following the general procedure, a mixture containing fluorescein monotrifluoromethanesulfonate (30 mg, 0.054 mmol), sSPhos (33 mg, 0.06 mmol), and [(1,5-COD)Pd(CH₂SiMe₃)₂] (25 mg, 0.06 mmol) was stirred at rt in tetrahydrofuran (1 mL) for 1 h. General work-up afforded 1a as a red solid (54 mg, 94%),

¹H NMR (400 MHz, CD₂Cl₂) δ 7.72-7.59 (m, 1H), 7.54-7.37 (m, 1H), 7.11 (d, J=8.0 Hz, 1H), 7.02 (dd, J=8.5, 1.9 Hz, 1H), 6.92 (dd, J=7.0, 2.5 Hz, 1H), 6.70 (d, J=8.5 Hz, 1H), 6.64 (t, J=1.6 Hz, 1H), 4.66 (dt, J=12.1, 6.1 Hz, 1H), 2.23-2.12 (m, 1H), 1.80 (br, 4H), 1.59 (br, 1H), 1.48 (s, 1H), 1.42 (d, J=6.0 Hz, 3H), 1.32-1.12 (m, 2H), 1.05 (dd, J=6.0, 3.7 Hz, 4H), 0.93 (t, J 7.1 Hz, 1H).

¹³C NMR (101 MHz, CD₂Cl₂) δ 159.08, 144.66, 144.48, 138.97, 138.93, 135.89, 135.12, 134.64, 134.15, 133.01, 132.66, 132.62, 132.51, 131.09, 130.96, 130.62, 130.60, 130.20, 12.9.17, 129.15, 126.40, 126.34, 126.06, 117.33, 107.47, 106.97, 71.09, 70.64, 34.12, 33.90, 33.63, 33.26, 28.23, 28.02, 27.71, 27.69, 27.35, 27.16, 27.03, 26.91, 26.84, 26.81, 26.72, 26.69, 26.03, 26.02, 22.33, 21.87, 21.38, 21.30, 13.81. (observed complexity is due to C—P coupling).

³¹P NMR (121 MHz, CD₂Cl₂) δ 31.99.

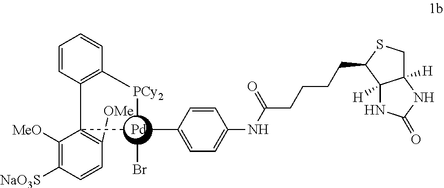

Following the general procedure, a mixture containing and bromide (biotin) (30 mg, 0.054 mmol), sSPhos (33 mg, 0.06 mmol), and [(1,5-COD)Pd(CH₂SiMe₃)₂] (25 mg, 0.06 mmol) was stirred at rt in tetrahydrofuran (1 mL) for 1 h. General work-up afforded 1b as a pink solid (54 mg, 99%).

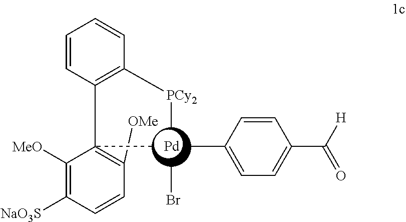

Following the general procedure, a mixture containing 4-chlorobenzaldehyde (8.4 mg, 0.054 mmol), sSPhos (31 mg, 0.06 mmol), and [(1,5-COD)Pd(CH₂SiMe₃)₂] (25 mg, 0.06 mmol) was stirred at rt in tetrahydrofuran (1 mL) for 1 h. General work-up afforded 1c as a white solid (36 mg, 89%).

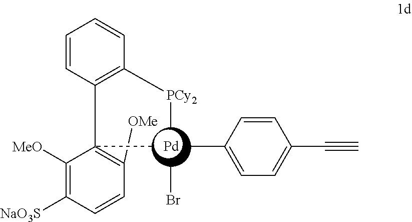

Following the general procedure, a mixture containing 4-bromophenylacetylene (9.7 mg, 0.054 mmol), sSPhos (31 mg, 0.06 mmol), and [(1,5-COD)Pd(CH₂SiMe₃)₂] (25 mg, 0.06 mmol) was stirred at rt in tetrahydrofuran (1 mL) for 1 h. General work-up afforded 1d as a yellow solid (36 mg, 89%).

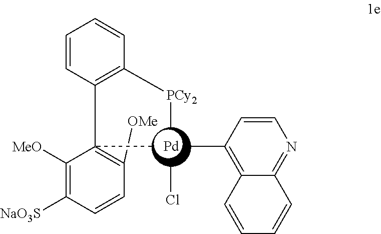

Following the general procedure, a mixture containing 4-chloroquinoline (9.8 mg, 0.054 mmol), sSPhos (31 mg, 0.06 mmol), and [(1,5-COD)Pd(CH₂SiMe₃)₂] (25 mg, 0.06 mmol) was stirred at rt in tetrahydrofuran (1 mL) for 1 h. General work-up afforded 1e as a light brown solid (40 mg, 94%).

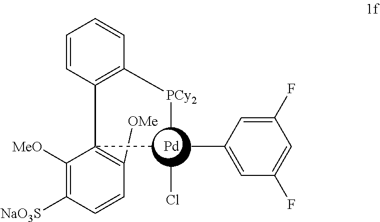

Following the general procedure, a mixture containing 1-Bromo-3,5-difluorobenzene (9.8 mg, 0.054 mmol), sSPhos (31 mg, 0.06 mmol), and [(1,5-COD)Pd(CH₂SiMe₃)₂] (25 mg, 0.06 mmol) was stirred at rt in tetrahydrofuran (1 mL) for 1 h. General work-up afforded 1f as a yellow solid (45 mg, 99%).

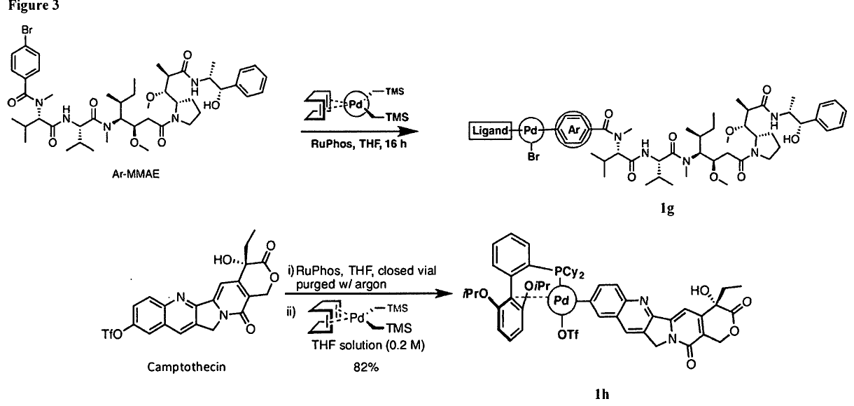

Following the general procedure, other reagents were also synthesized (FIG. 3).

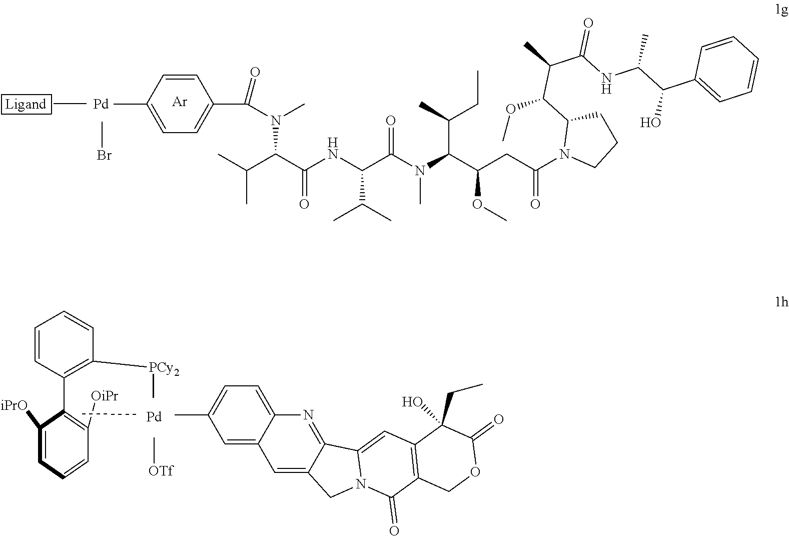

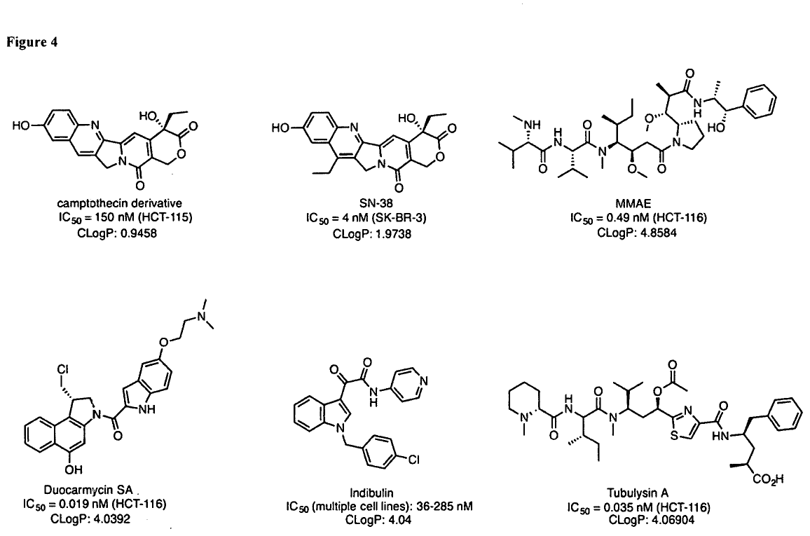

After purging with argon, a scintillation vial (10 mL), which was equipped with a magnetic stir bar, was charged with RuPhos (1 equiv), the aryl containing drug or the drag derivative, and [(1,5-COD)Pd(CH₂SiMe₃)₂] (1.1 equiv) dissolved in tetrahydrofuran (THF, 0.2 M). The closed vial was purged with argon and stirred for 16 h. The resulting precipitate was filtered, washed with pentane (3×3 mL), and dried under reduced pressure to afford the oxidative addition complex (FIG. 3). Other potential and containing drugs or drug derivatives are shown in FIG. 4.

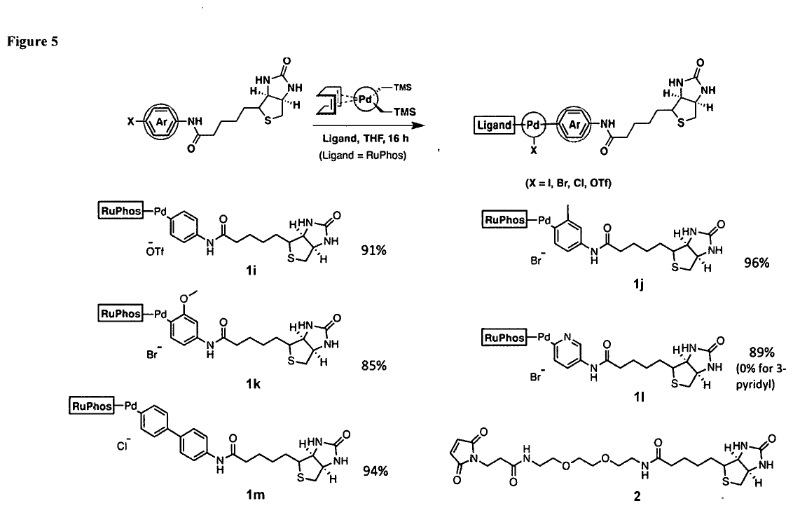

Aryl link biotin conjugates 1i-1m were synthesized under similar conditions (FIG. 5).

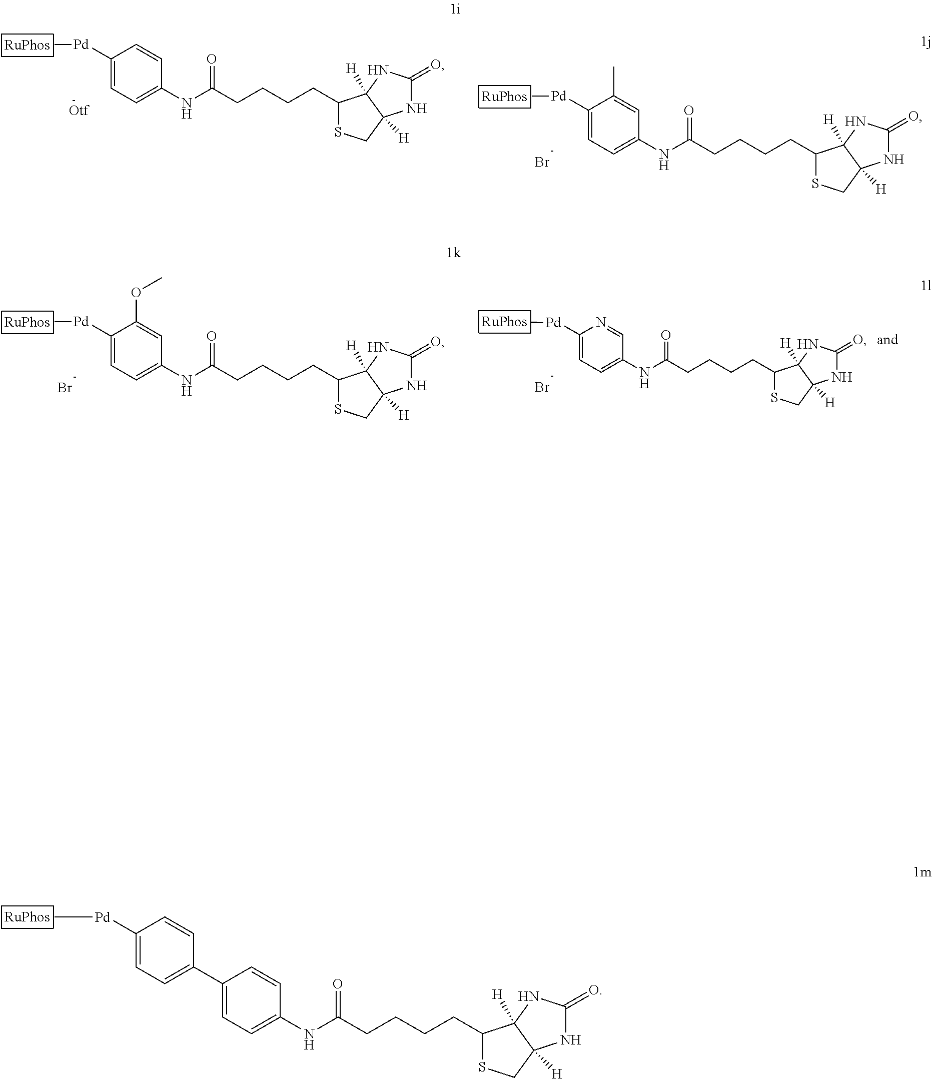

### Example 2—Model Polypeptide

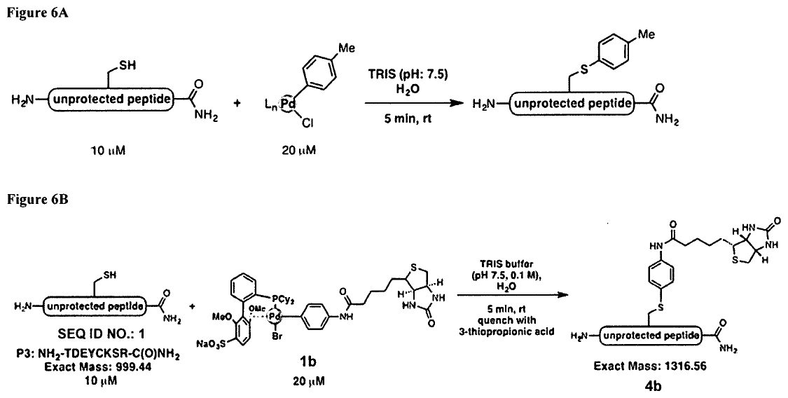

A sulfonated ligand, sodium 2′-dicyclohexylphosphino-2,6-dimethoxy-1,1′-biphenyl-3-sulfonate hydrate (sSPhos) was synthesized, and this ligand has been utilized in the context of Suzuki-Miyaura couplings in water.¹² Both a model peptide and a designed ankyrin repeat protein (DARPin), bearing a single cysteine residue, respectively, were exposed to an sSPhos supported palladium reagent in a completely aqueous environment (FIG. 6A and).¹¹ In both cases, the product of cysteine arylation was formed rapidly at room temperature with high chemoselectivity (99% yield). In contrast, use of the complexes supported by RuPhos and SPhos ligands provided <20% yield (18% and 14%, respectively). These results are consistent with the idea that tire sulfonate group of sSPhos allows the palladium reagent to be more soluble in water and therefore able to react with cysteine residues more efficiently.

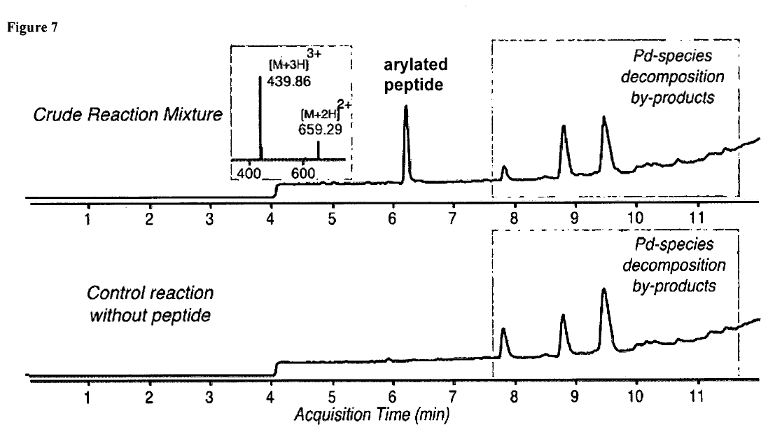

Each of the reagents shown in FIG. 2, regardless of complexity, displayed similar reaction profiles with biomolecules containing cysteine (FIG. 6), revealing the generality of this palladium-mediated S-arylation approach. The clean reaction profiles resulted in easily purified products with <1% Pd remaining. For example, reaction between 1b and an unprotected model polypeptide P3 (FIG. 6) resulted in a complete conversion of the starting peptide material as suggested by LC-MS analysis of the reaction mixture. The model polypeptide had the sequence NH₂-TDEYCKSR-C(O)NH₂ (SEQ ID NO: 3). Importantly, only Cys S-arylated product 4b was observed as a result of this transformation in combination with several decomposition products of 1b produced upon quenching with acid present in the LC-MS running solvent mixture (FIG. 7). These decomposition products eluted significantly later relatively to the peptide product 4b.

### General Linear Peptide Synthesis Procedure:

All peptides were synthesized on a 0.2 mmol scale using manual Fmoc-SPPS chemistry under flow using a 3 min cycle for each amino acid.¹³ Specifically, all reagents and solvents wore delivered to a stainless steel reactor containing resins at a constant flow rate using HPLC pump; the temperature of the reactor was maintained at 60° C. during the synthesis using a water bath. The procedure for each amino acid coupling cycle included: 1) a 30 s coupling with 1 mmol of the corresponding Fmoc-protected amino acid, 1 mmol HBTU, and 500 μL of diisopropyl ethyl amine (DIPEA) in 2.5 ml, of DMF at a flow rate of 6 mL/min (note that for the coupling of cysteine and tryptophan, 190 μL of DIPEA was used to prevent racemization); 2) 1 min wash with DMF at a flow rate of 20 mL/min; 3) 20 s deprotection with 50% (v/v) piperidine in DMF at a flow rate of 20 mL/min; and 4) 1 min wash with DMF at a flow rate of 20 mL/min. After completion of the stepwise SPPS, the resin was washed thoroughly with DCM and dried under vacuum. The peptide was simultaneously cleaved from tire resin and deprotected on the side-chains by treatment with 2.5% (v/v) water, 2.5% (v/v) 1,2-ethanedithiol (EDT), and 2.5% (v/v) triisopropryisilane in neat trifluoroacetic acid (TFA) for 7 min at 60° C. The resulting solution was then triturated and washed with cold diethyl ether three times. The obtained solid was dissolved in 50% H₂O: 50% acetonitrile containing 0.1% TFA and lyophilized. The following peptides were synthesized with tins procedure:

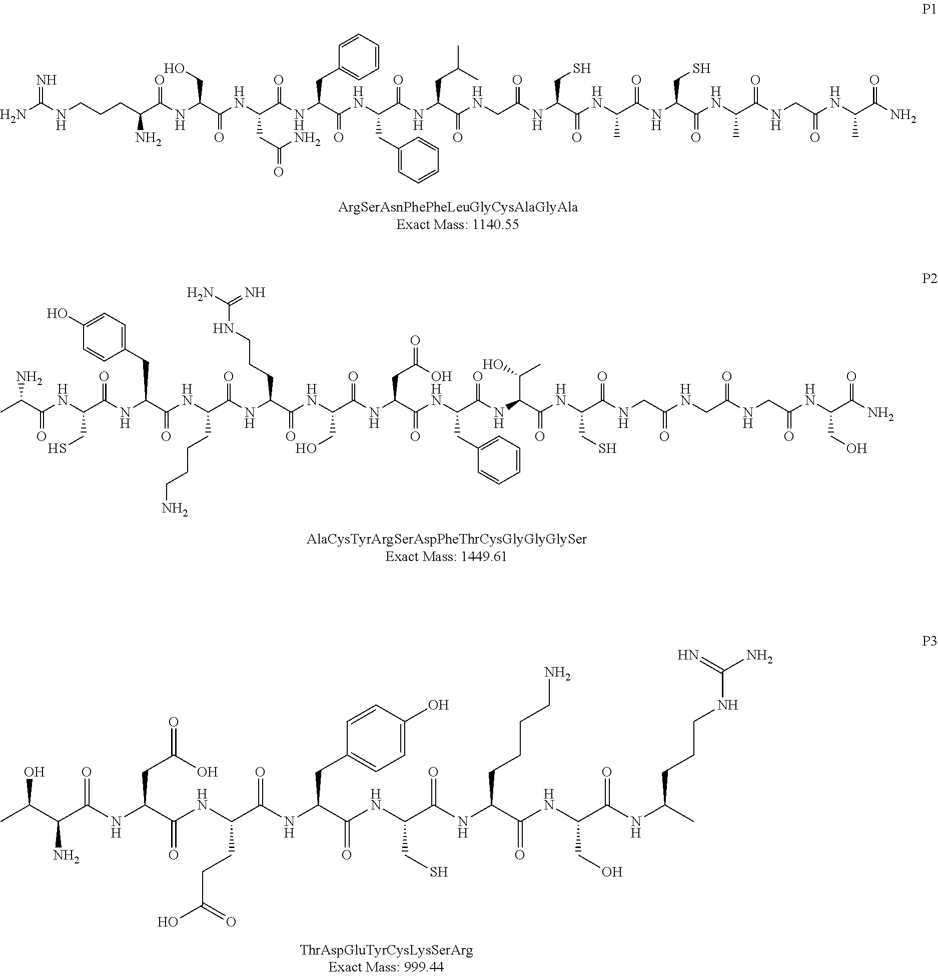

### Peptide Purification

Solvent compositions for RP-HPLC purification are water with 0.1% TFA (solvent C) and acetonitrile with 0.1% TFA (solvent D). The crude peptide was dissolved in 50% C: 50% D and purified by semi-preparative RP-HPLC (Agilent Zorbax 300SB Cos column: 21.2×250 mm, 7 μm, linear gradient: 5-50% B over 65 min, flow rate: 5 mL/min). Each HPLC fraction was analyzed by mass-directed preparative LC-MS. HPLC fractions containing pure product were further confirmed by LC-MS, combined, and lyophilized. Peptides synthesized using manual SPPS and purified by RP-HPLC are listed in Table 1.

TABLE 1
Sequences and masses of peptides synthesized
by manual fast flow SPPS.

| | SEQ | | Calculated | Observed Mass |
|---|---|---|---|---|
| Peptide | ID NO. | Sequence | mass | [M + H]⁺ |
| P1 | 1 | NH₂-RSNFFLGCAGA-C(O)NH₂ | 1140.55 | 1141.55 |
| P2 | 2 | NH₂-ACYKRSDFTCGGGS-C(O)NH₂ | 1449.61 | 1450.63 |
| P3 | 3 | NH₂-TDEYCKSR-C(O)NH₂ | 999.44 | 1000.42 |

### General Bioconjugation Procedure A

A solution of the Pd reagent in water (40 μM) was added to a solution of peptide P3 (20 μM) in Tris buffer (0.1 M, pH 7.5). Note: if the palladium reagent was not readily soluble in H₂O, the slurry was sonicated for 10 s to facilitate this process. Final conditions: [Pd]=20 μM: [peptide]=10 μM; After 5 min at rt, 3-mercaptopropionic acid (3 equiv to the palladium complex, solution in 6 μL of H₂O) was added to the reaction mixture to quench the remaining palladium species. The reaction was allowed to stand for 5 min and subsequently characterized by LC-MS.

### Exemplary Arylated Peptides

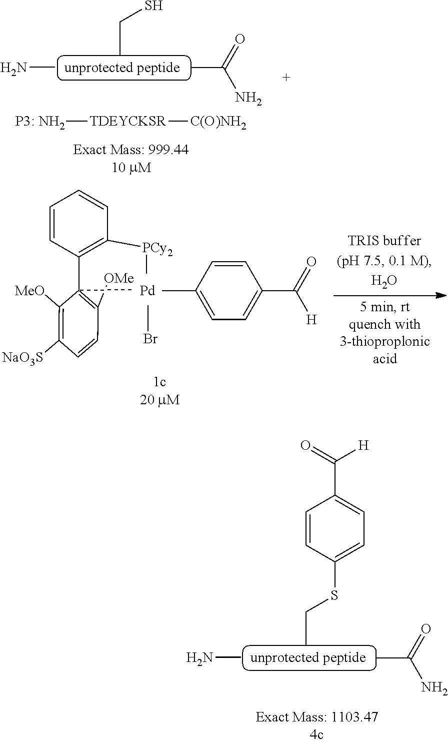

The arylated peptide 4c was synthesized according to general procedure A using the scheme above.

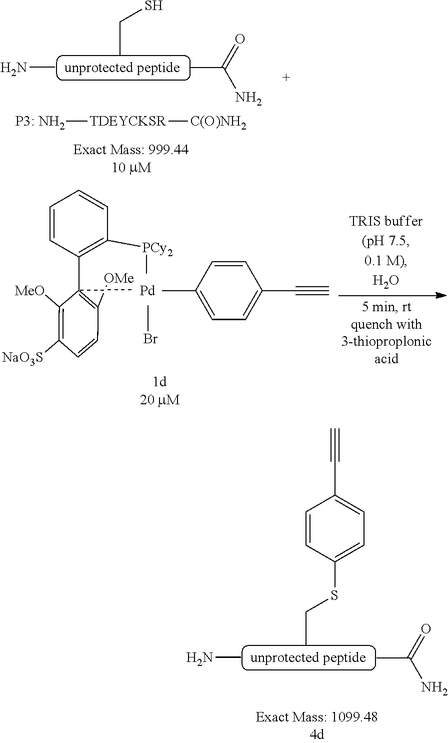

The arylated peptide 4d was synthesized according to general procedure A using the scheme above.

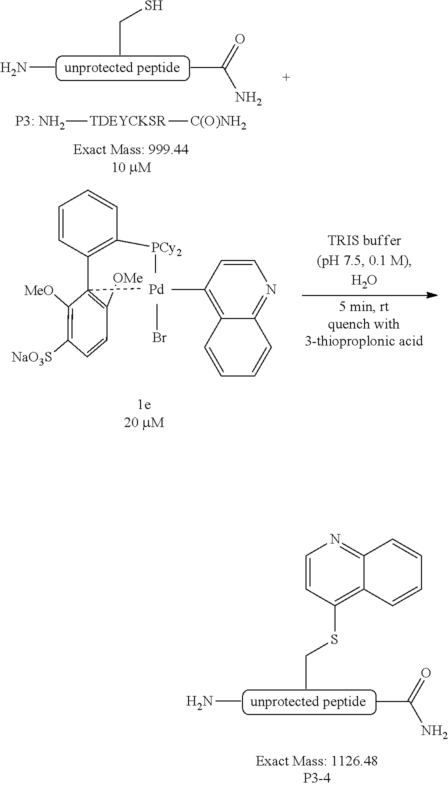

The arylated peptide 4e was synthesized according to general procedure A using the scheme above.

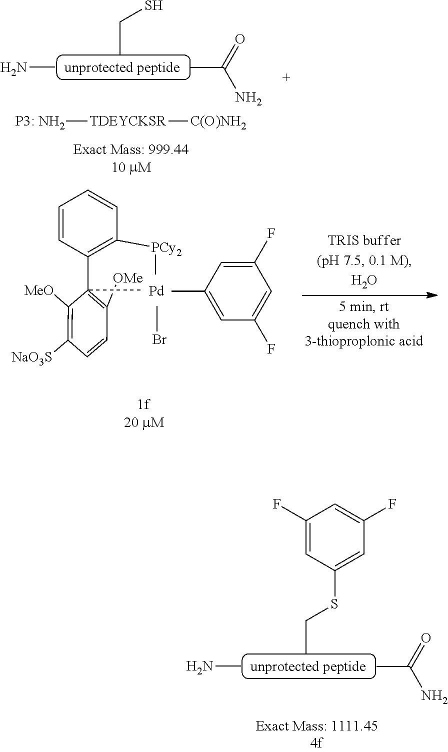

The arylated peptide 4f was synthesized according to general procedure A using the scheme above.

### Example 3—Conjugating Drug Molecules to Antibody by Palladium Reagents

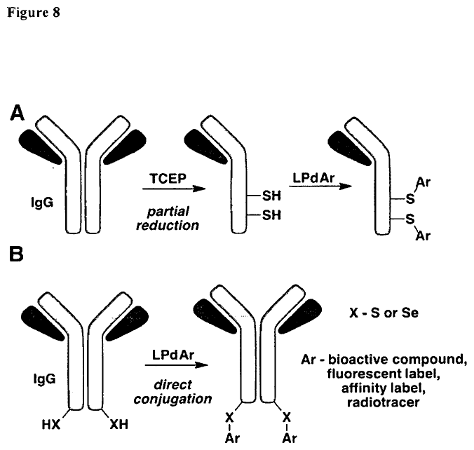

Further studies were aimed at functionalization of native Cys residues in IgG antibodies. Specifically, two independent approaches were examined, where one can either functionalize native Cys moieties after partial antibody reduction or perform functionalization on the intact antibody containing single-point mutation with Cys or selenocysteine moieties on the main-chain terminus (FIG. 8). In both cases, the resulting constructs are significantly more chemically stable towards degradation than their alkyl, disulfide and maleimide congeners. This stability enhancement along with the highly selective and rapid bioconjugation conferred by Pd(II) reagents provided significantly improved handling capabilities and expanded therapeutic properties for the resulting anti body-drag conjugates.

### Exemplary Antibody-Drug Conjugates

### Reduction of the Antibody

Tris buffer (6.5 μL, 1.0 M, pH 8.0) and TCEP (4.2 μL, 25 mM in water, pH 6.7; 24.0 equiv) were added to a solution of trastuzumab (57 μM, 76.6 μL) in PBS. The reaction mixture was pipetted up mid down 20 times. The vial was incubated in a water bath at 37° C. for 2.0 h. The final reaction conditions for the reduction were 50 μM antibody with 24.0 equivalents of TCEP.

### Formation of a C—S Bond

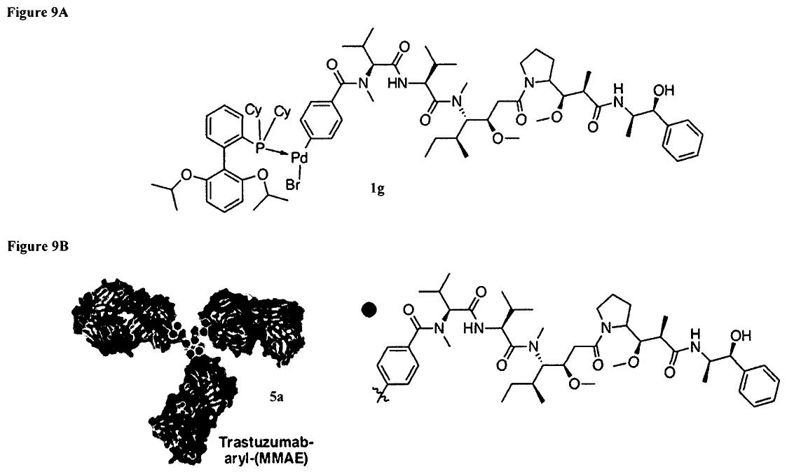

The reaction mixture was transferred onto 30k spin filter (4 mL, Amicon) and diluted 10 fold with 1.0 mM EDTA (in PBS, pH=8.0) followed by centrifugation (4000 rpm×11 minutes, 4° C.). The 10-fold dilution and concentration steps were repeated twice more. Finally, approximately 110 μL residue was transferred to a 1.7 mL eppendorf tube for conjugation. Tris buffer (245.5 μL, 0.1 M, pH 7.0) and palladium-MMAE complex Ig (87.3 μL, 2.0 mM m DMSO) were added to the partially reduced antibody. The reaction mixture was pipetted up and down 20 times, and the reaction mixture was left at room temperature for 18 hours. The final reaction conditions for the coupling were 12 μM antibody with 5.0 equivalents of Pd complex Ig (FIGS. 9A and 9B)

The crude reaction mixture was quenched with a solution of thiopropionic acid (35 μL, 50 mM in Tris buffer, 10 equivalents relative to the amount of the palladium reagent used), and the resulting solution was incubated at room temperature for 5.0 minutes. The solution was diluted with 4.0 mL phosphate buffer saline (1×PBS) and filtered through 0.2 μm nylon spin filter (PALL Life Sciences) into 15 ml, tube with 30K filter. The tube was capped and inverted-reverted 30 times followed by subjection to centrifugation (12 minutes, 4000 rpm). The filtrate (aqueous) was removed and the 30K filter tube was re-filled with PBS, capped, inverted-reverted, and subjected to centrifugation (11 minutes, 4000 rpm). The aforementioned procedure was repeated twice. The concentration of the final solution was determined by UV absorbance. An aliquot was removed for reduction with TCEP (200 μM, pH=7.0) and LC-MS analysis to determine identity and drug-antibody ratio. The final product solution was flash-frozen (liquid N₂:−196° C.) and stored at −80° C. Based on LCMS analysis, the drug-to-antibody ratio (DAR) was calculated to be about 8.0. The concentration by UV absorbance was 1.337 mg/mL, and there was >90% conversion to the target antibody-drug conjugate (ADC) 5a,

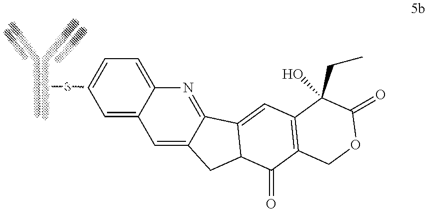 [SMILES: CC[C@@]1(O)C(=O)OCC2=C1C=C1c3nc4ccccc4cc3CC1C2=O]

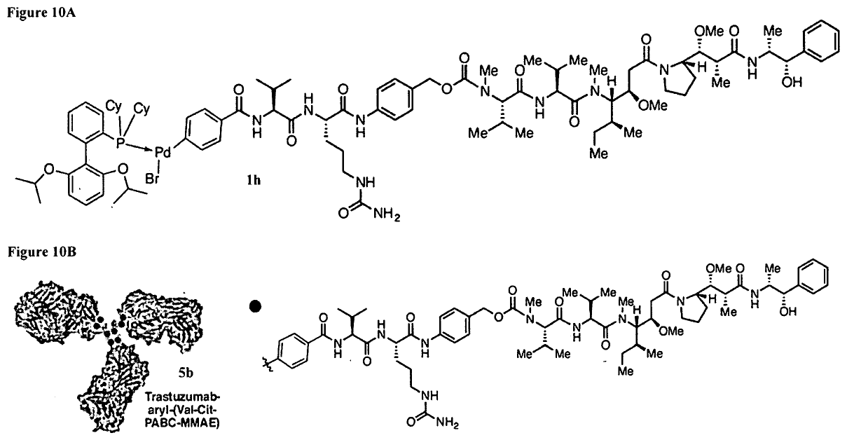

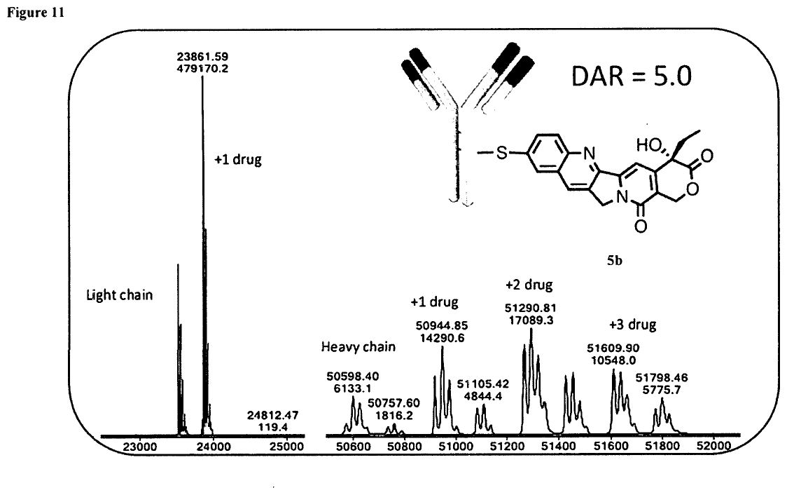

Following the protocol used for ADC 5a, a camptothecin derivative was synthesized using Pd complex 1h (FIGS. 10A and 10B). Based on LCMS analysis, the drug-to-antibody ratio (DAR) was calculated to be about 5.0 for ADC 5b as shown in FIG. 11.

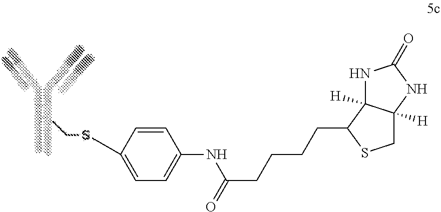 [SMILES: Cc1ccc(NC(=O)CCCCC2SC[C@@H]3NC(=O)N[C@H]23)cc1]

Following a protocol similar to the one used for ADC 5a, a biotin derivative was synthesized using Pd complex 1i in DMF for 30 min. Based on LCMS analysis, the drug-to-antibody ratio (DAR) for 5c was calculated to be about 5.9 (data not shown).

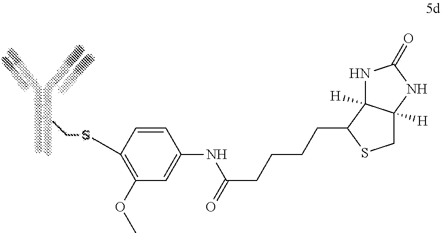 [SMILES: COc1cc(NC(=O)CCCCC2SC[C@@H]3NC(=O)N[C@H]23)ccc1C]

Following a protocol similar to the one used for ADC 5a, a biotin derivative was synthesized using Pd complex 1i in DMF for 30 mm. Based on LCMS analysis, the drug-to-antibody ratio (DAR) for 5d was calculated to be about 5.4 (data not shown).

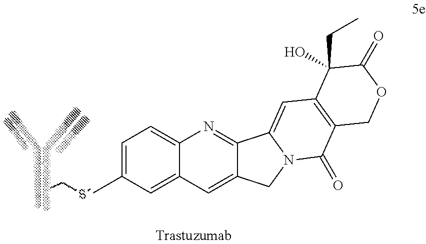 [SMILES: CC[C@@]1(O)C(=O)OCc2c1cc1n(c2=O)Cc2cc3cc(C)ccc3nc2-1]

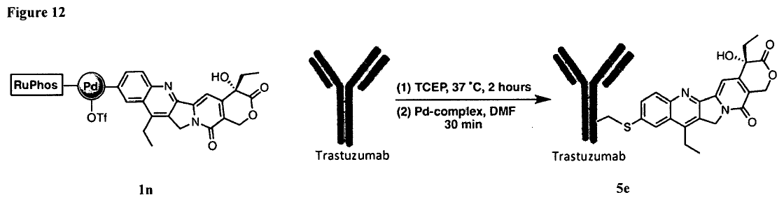

Following a protocol similar to the one used for ADC 5a, a biotin derivative was synthesized using Pd complex 1i in DMF for 30 min (FIG. 12). Based on LCMS analysis, the drug-to-antibody ratio (DAR) for 5e was calculated to be about 2.0 (data not shown).

### Example 4—Stability of Antibody Conjugates

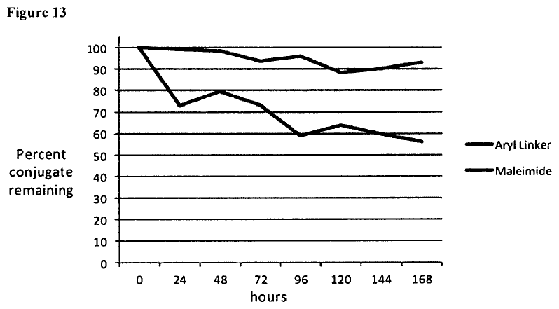

An aryl linked biotin derivative conjugated to an antibody displayed increased human blood plasma stability compared to a maleimide antibody analog (FIG. 13). The concentration of the conjugate in pooled human blood plasma was 1 μM, and the samples were incubated at 37° C. for 7 days (168 h).

### Example 5—Synthesis of Polymetallic Species

### General Procedures for the Synthesis of Bis-Palladium Oxidative Addition Complexes

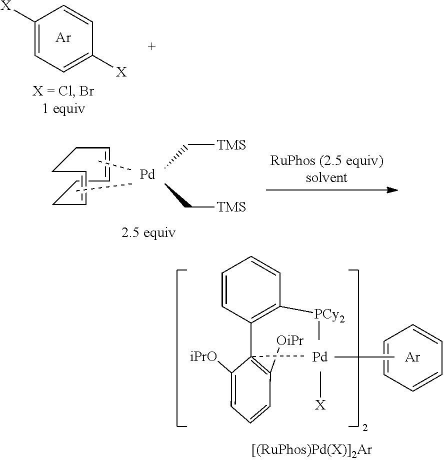

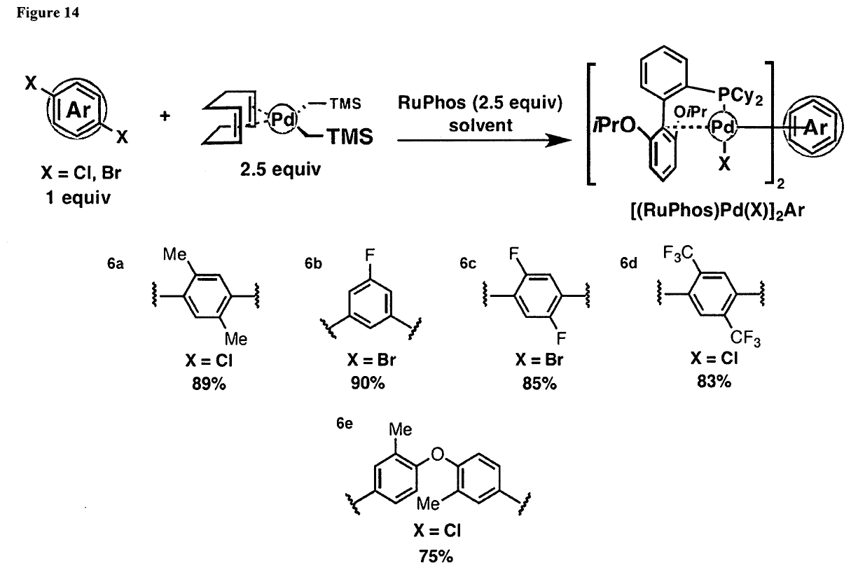

Palladium reagents were synthesized following the scheme of FIG. 14 for general procedure A. In a nitrogen-filled glovebox, an oven-dried scintillation vial (10 mL), which was equipped with a magnetic stir bar and fitted with a Teflon screwcap septum, was charged with RuPhos (2.5 equiv), a dihaloaryl compound (1 equiv) and cyclohexane (1.2 mL). Solid [(1,5-COD)Pd(CH₂SiMe₃)₂] (2.5 equiv) was added rapidly in one portion and the resulting solution was stirred for 16 h at it. After this time, pentane (3 mL) was added and the resulting mixture was placed into a −20° C. freezer for 3 h. The vial was then taken outside of the glovebox, and the resulting precipitate was filtered, washed with pentane (3×3 mL), and dried under reduced pressure to afford the oxidative addition complex (FIG. 14).

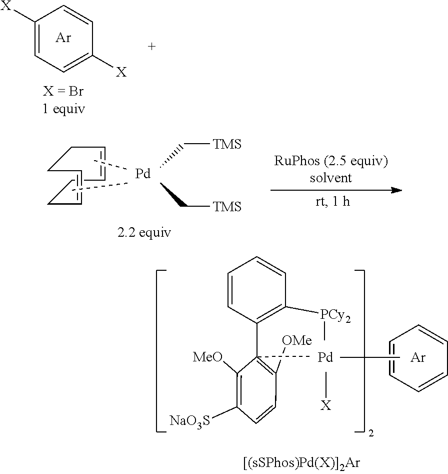

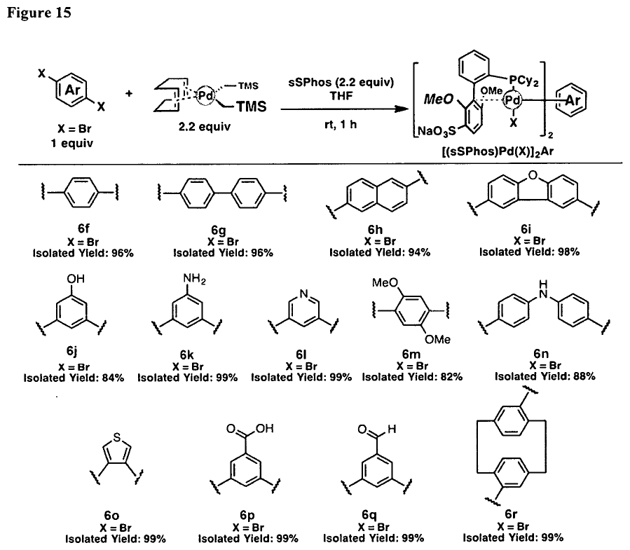

Palladium reagents were synthesized following the scheme of FIG. 15 for general procedure B. A scintillation vial (10 mL), equipped with a magnetic stir bar, was charged with sSPhos (2.2 equiv), Ar—X (1 equiv), and tetrahydrofuran. Solid [(1,5-COD)Pd(CH₂SiMe₃)₂] (2.2 equiv) was added rapidly in one portion and the resulting solution was stirred for 1 h at rt. After this time, pentane (3 mL) was added and the resulting mixture was placed into a −20° C. freezer for 2 h. The vial was removed from the freezer and, in the air, the resulting precipitate was filtered, washed with pentane (5×3 mL), and dried under reduced pressure to afford the oxidative addition complex (FIG. 15).

### Exemplary Oxidative Addition Complexes

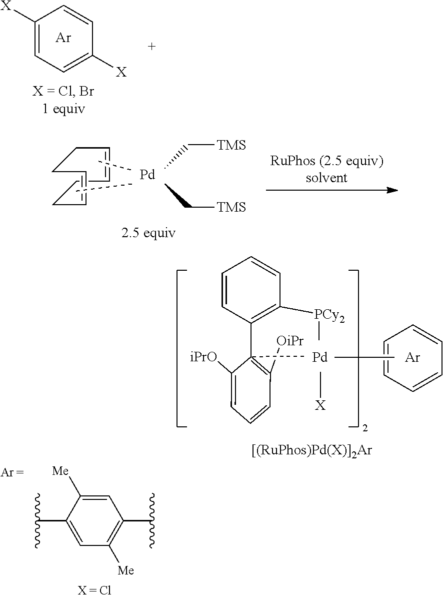

Following general procedure A, compound 6a was synthesized with 89% yield.

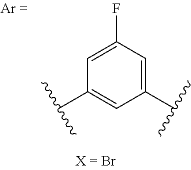 [SMILES: *c1cc(*)cc(F)c1]

Following general procedure A, compound 6b was synthesized with 90% yield.

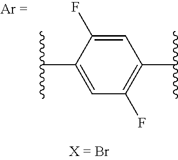 [SMILES: *c1cc(F)c(*)cc1F]

Following general procedure A, compound 6c was synthesized with 85% yield.

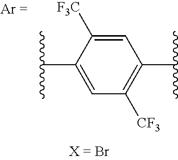 [SMILES: *c1cc(C(F)(F)F)c(*)cc1C(F)(F)F]

Following general procedure A, compound 6d was synthesized with 83% yield.

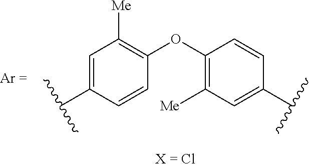 [SMILES: *c1ccc(Oc2ccc(*)cc2C)c(C)c1]

Following general procedure A, compound 6e was synthesized with 75% yield.

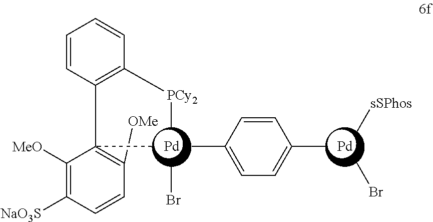

Following general procedure B, a mixture containing 1,4-dibromobenzene (12.7 mg, 0.054 mmol), sSPhos (61 mg, 0.119 mmol), and [(1,5-COD)Pd(CH₂SiMe₃)₂] (46 mg, 0.119 mmol) was stirred at rt in tetrahydrofuran (1.5 mL) for 1 h. General work-up afforded 6f as a brown solid (76 mg, 96%).

¹H NMR (400 MHz, CD₂Cl₂) δ 7.72-7.59 (m, 1H), 7.54-7.37 (m, 1H), 7.11 (d, J=8.0 Hz, 1H), 7.02 (dd, J=8.5, 1.9 Hz, 1H), 6.92 (dd, J=7.0, 2.5 Hz, 1H), 6.70 (d, J=8.5 Hz, 1H), 6.64 (t, J=1.6 Hz, 1H), 4.66 (dt, J=12.1, 6.1 Hz, 1H), 2.23-2.12 (m, 1H), 1.80 (br, 4H), 1.59 (br, 1H), 1.48 (s, 1H), 1.42 (d, J=6.0 Hz, 3H), 1.32-1.12 (m, 2H), 1.05 (dd, J=6.0, 3.7 Hz, 4H), 0.93 (t, J=7.1 Hz, 1H).

¹³C NMR (101 MHz, CD₂Cl₂) δ 159.08, 144.66, 144.48, 138.97, 138.93, 135.89, 135.12, 134.64, 134.15, 133.01, 132.66, 132.62, 132.51, 131.09, 130.96, 130.62, 130.60, 130.20, 129.17, 129.15, 126.40, 126.34, 126.06, 117.33, 107.47, 106.97, 71.09, 70.64, 34.12, 33.90, 33.63, 33.26, 28.23, 28.02, 27.71, 27.69, 27.35, 27.16, 27.03, 26.91, 26.84, 26.81, 26.72, 26.69, 26.03, 26.02, 22.33, 21.87, 21.38, 21.30, 13.81. (observed complexity is due to C—P coupling).

³¹P NMR (121 MHz, CD₂Cl₂) δ 31.99.

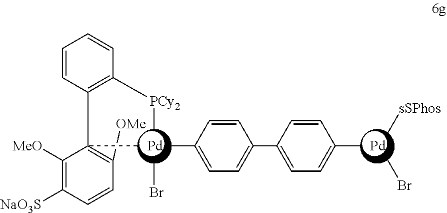

Following general procedure B, a mixture containing 4,4-Dibromobiphenyl (16.8 mg, 0.054 mmol), sSPhos (61 mg, 0.119 mmol), and [(1,5-COD)Pd(CH₂SiMe₃)₂] (46 mg, 0.119 mmol) was stirred at rt in tetrahydrofuran (1.5 mL) for 1 h. General work-up afforded 6g as a brown solid (81 mg, 96%).

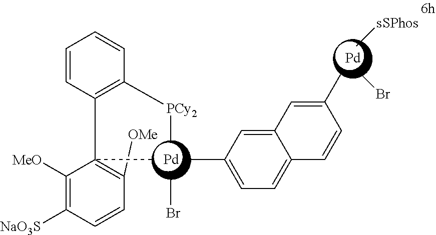

Following general procedure B, a mixture containing 2,6-Dibromonaphthalene (12.7 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6h as a light red solid (63 mg, 94%).

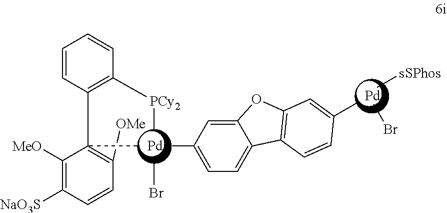

Following general procedure B, a mixture containing 2,8-dibromodibenzofuran (14.3 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6i as a brown solid (67.3 mg, 98%).

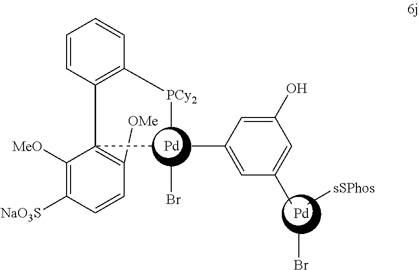

Following general procedure B, a mixture containing 3,5-Dibromophenol (11.2 mg, 0.044 mmol), sSPhos (50 mg, 0.01 mmol), and [(1,5-COD)Pd(CH₂SiMe₃)₂] (38 mg, 0.0.1 mmol) was stirred at rt in tetrahydrofuran (1.5 mL) for 1 h. General work-up afforded 6j as a light red solid (55 mg, 86%).

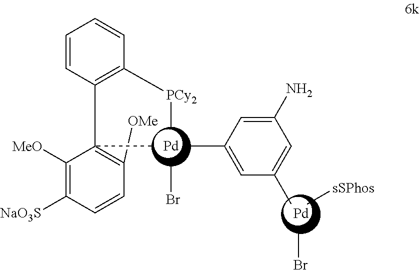

Following general procedure B, a mixture containing 3,5-Dibromoaniline (11.2 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6k as a brown solid (68 mg, 99%).

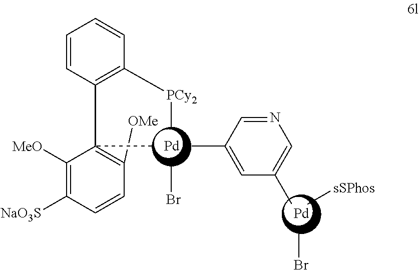

Following general procedure B, a mixture containing 3,5-Dibromopyridine (10.5 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6l as a brown solid (65 mg, 99%).

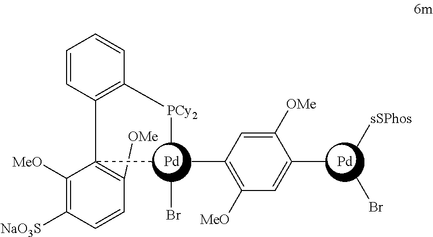

Following general procedure B, a mixture containing 1,4-Dibromo-2,5 dimethoxybenzene (13.1 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6m as a brown solid (56 mg, 84%).

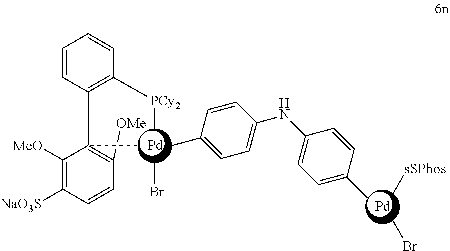

Following general procedure B, a mixture containing Bis(4-bromophenyl)amine (14.5 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6n as a brown solid (61 mg, 90%).

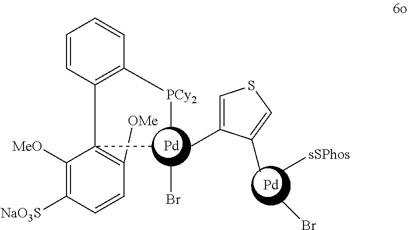

Following general procedure B, a mixture containing 3,4-Dibromothiophene (5 μL, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6o as a brown solid (57 mg, 89%).

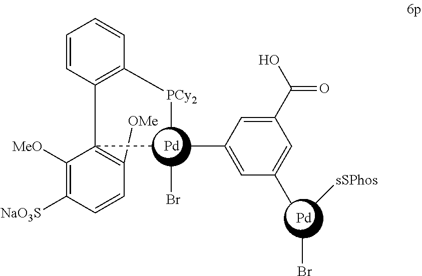

Following general procedure B, a mixture containing 3,5-Dibromobenzoic acid (12.3 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6p as a light brown solid (72 mg, 99%).

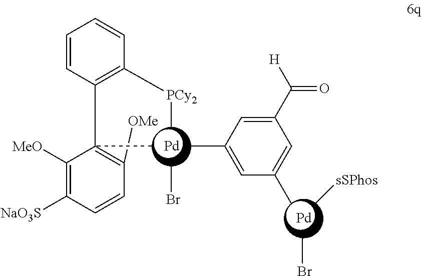

Following general procedure B, a mixture containing 3,5-Dibromobenzaldehyde (11.6 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6q as a light brown solid (66 mg, 99%).

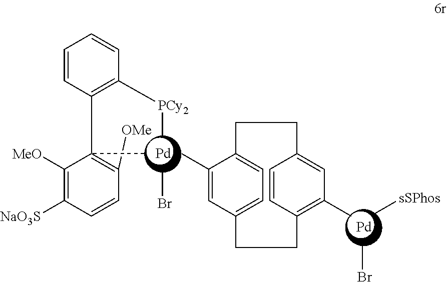

Following general procedure B, a mixture containing 5,11-dibromotricyclo[8.2.2.2˜4,7˜]hexadeca-1(12),4,6,10,13,15-hexaene (16.1 mg, 0.044 mmol), sSPhos (50 mg, 0.01 mmol), and [(1,5-COD)Pd(CH₂SiMe₃)₂] (38 mg, 0.01 mmol) was stirred at rt in tetrahydrofuran (1.5 mL) for 1 h. General work-up afforded 6r as a yellow solid (70 mg, 99%).

### Example 6—Stapling

Peptide macrocyclization is a burgeoning area in chemical biology and medicinal therapeutic development.^{13,14} Compared to their linear counterparts, peptide macrocycles have been shown to exhibit enhanced ceil permeability,¹⁵ increased target binding affinity,¹⁶ and resistance to proteolytic degradation.¹⁷ As such, several methods have been developed to bridge natural and unnatural amino acid residues along a peptide chain.¹⁸ Notably, many of these methods utilize the same cross-linker structures with few examples reported that introduce structural diversity at a late stage.¹⁹

Bis-palladium reagents ([(sSPhos)Pd(X)]₂Ar) were prepared of varying structure, functional group presence, substitution pattern, and conformation (Scheme t) from commercially available bis-aryl halides. High functional group tolerance and ease of isolation and purification were observed for all bis-palladium Macrocyclization reagents (82-99% yield).

The high functional group tolerance of the cysteine arylation method may provide a general method for the tuning of macrocyclic peptides. To test the efficiency of peptide macrocyclization using sSPhos supported reagents, a model peptide (P2) was prepared with two cysteines separated in an i, i+7 position. After dissolving the peptide in water and tris(hydroxymethyl)aminomethane (TRIS, pH 7.5), the palladium reagents were introduced in an aqueous solution, and the mixture was gently vortexed for five seconds. The reactions were allowed to proceed for ten minutes at room temperature, followed by addition of 3-thiopropionic acid to quench the reaction. LC-MS analysis indicated that most of the macrocyclization reactions were high yielding under these standard conditions (Scheme 1).

However, some peptide macrocycles (P2-8, 9, 10, 15, 16, and 19) required a small amount of acetonitrile (15% by volume) to reach full conversion, without which product formation was capped around 60%. The hydrophobic nature of some of the corresponding palladium reagents may require small amounts of acetonitrile to fully dissolve. The macrocyclic peptide products are readily purified through reverse-phase high performance liquid chromatography. ICP-MS analysis of tire peptides showed that more than 99% of palladium was removed during the purification process. No side reactions forming peptide oligomers through intermolecular cross-linking were observed.

### General Procedure A for Stapling Peptides

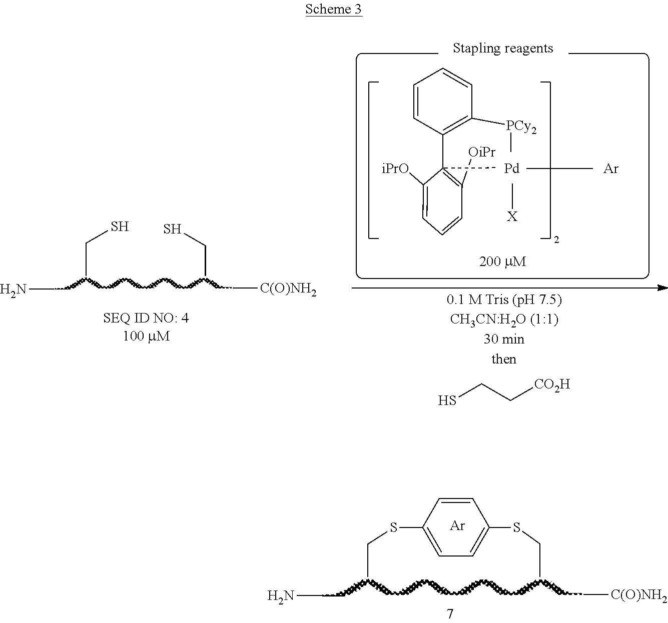

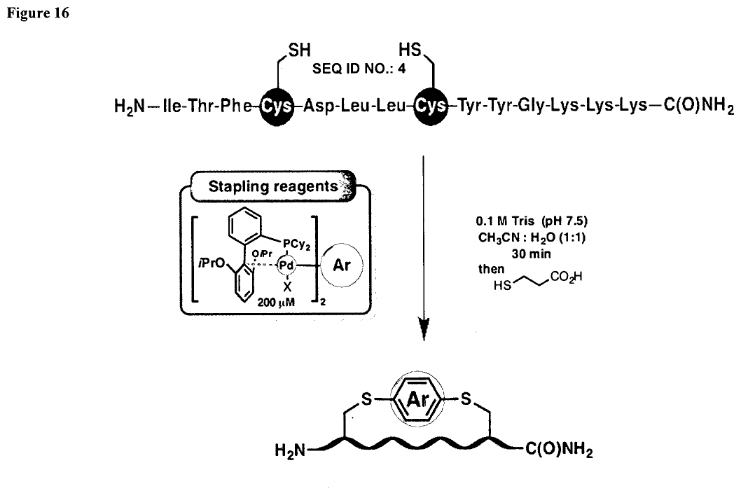

A scheme for stapling peptides using the bis-palladium compounds disclosed herein is shown in FIG. 16. The peptide had an amino acid sequence of NH₂-ITFCDLLCYYGKKK-CONH₂ (SEQ ID NO: 4), Peptide (4 μL, 1.5 mM), H₂O (23 μL), and Tris buffer (3 μL, 1 M, pH=7.5) were combined in a 0.6 mL plastic Eppendorf tube and the resulting solution was mixed using a vortexer. A stock solution of the palladium complex (30 μL, 400 μM) in H₂O was added in one portion. The palladium complexes were dissolved in acetonitrile solution. The reaction tube was vortexed to ensure proper reagent mixing and left at room temperature for 30 min. The reaction was quenched by the addition of 3-mercaptopropionic acid (6.3 μL, 1 μL/mL solution). After an additional 5 min the LCMS solution (60 μL) was added to the Eppendorf and the reaction mixture was analyzed by LCMS.

Final concentration of the reaction before quenching:

peptide—100 μM,

Pd complex—200 μM,

Tris buffer—100 mM.

CH₃CN:H₂O=1:1.

### General Procedure B for Stapling Peptides

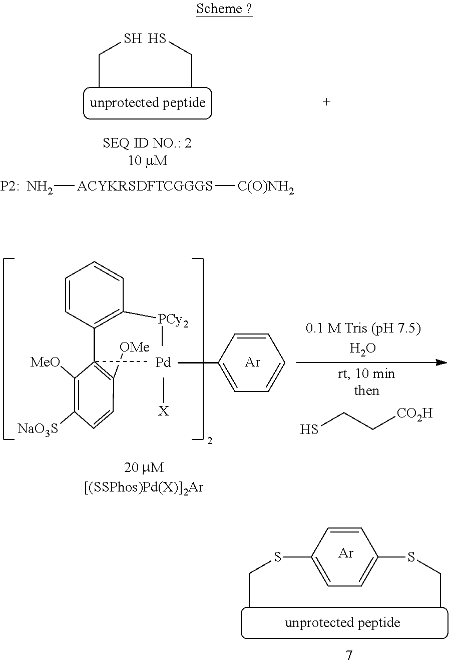

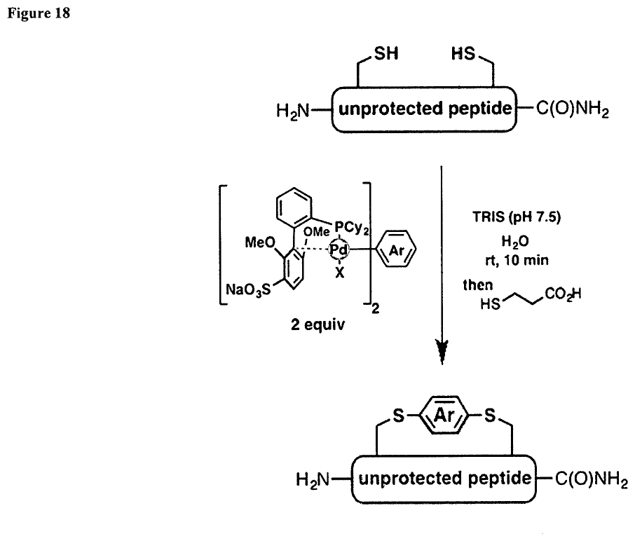

A scheme for stapling peptides using the bis-palladium compounds disclosed herein is shown in FIG. 18. The peptide P2 had an amino acid sequence of NH₂-ACYKRSDFTCGGGS-CONH₂ (SEQ ID NO: 2). A solution of the Pd reagent in water (40 μM) was added to a solution of peptide (20 μM) in Tris buffer (0.1 M, pH 7.5). Note: if the palladium reagent was not readily soluble in H₂O, the slurry was sonicated for 10 s to facilitate this process. Final conditions: [Pd]=20 μM; [peptide]=10 μM; After 10 min stirring at rt, 3-mercaptopropionic acid (3 equiv to the palladium complex, solution in 6 μL of H₂O) was added to the reaction mixture to quench the remaining palladium species. The reaction was allowed to stand for 5 min. The crude peptide was purified using preparative HPLC as described above or immediately injected for LC-MS analysis.

### Exemplary Stapled Peptides

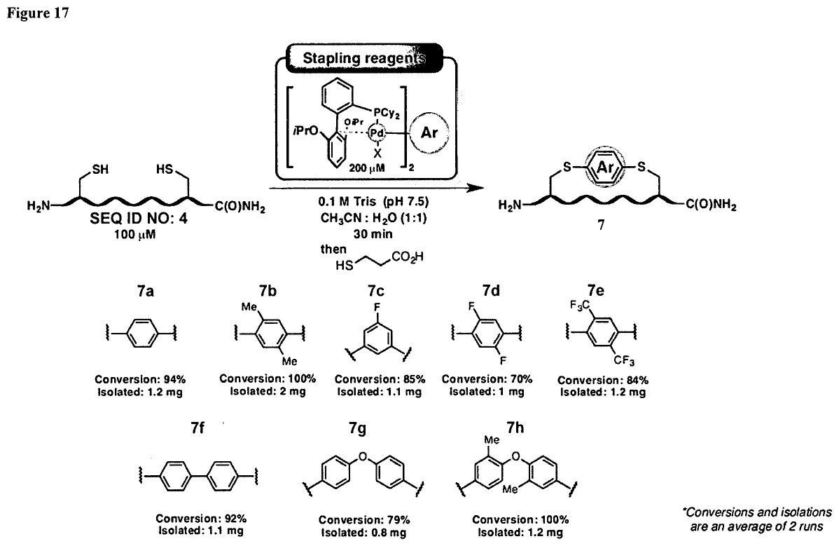

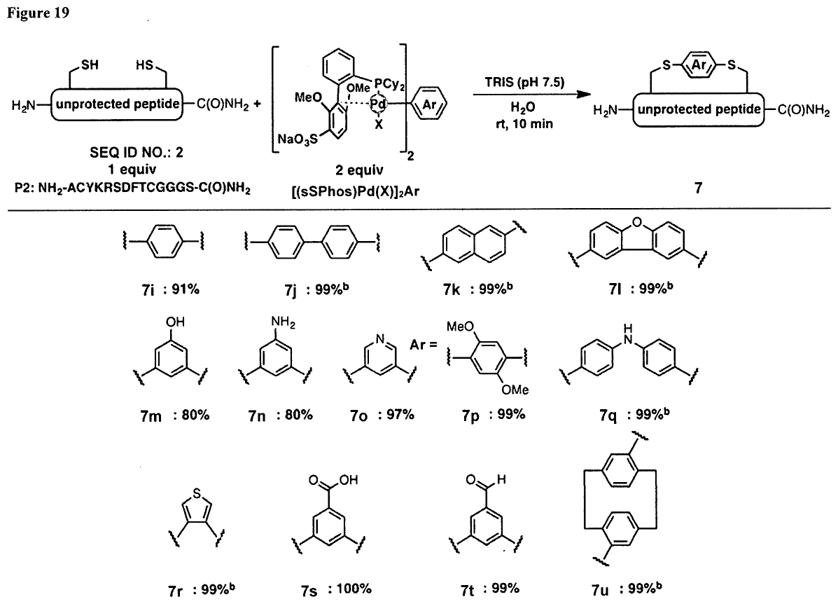

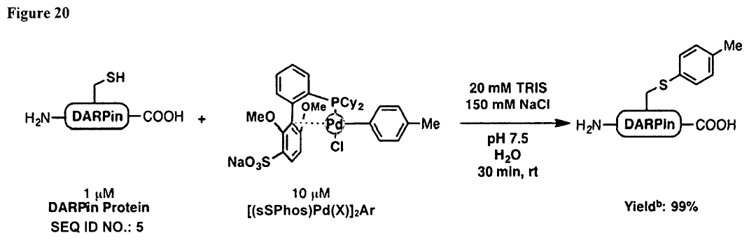

Exemplary stapled peptides are shown in FIGS. 17 and 19.

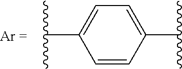 [SMILES: *c1ccc(*)cc1]

The stapled peptide 7a was synthesized according to general procedure A (1.2 mg, 94%). Final conditions before quenching: peptide—100 μM, palladium reagent—200 μM, 0.1 M Tris (pH 7.5), CH₃CN:H₂O=1:1.

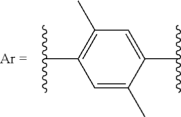 [SMILES: *c1cc(C)c(*)cc1C]

The stapled peptide 7b was synthesized according to general procedure A (2 mg, 100%). Final conditions before quenching: peptide—100 μM, 6a—200 μM, 0.1 M Tris (pH 7.5), CH₃CN:H₂O=1:1.

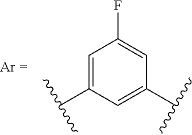 [SMILES: *c1cc(*)cc(F)c1]

The stapled peptide 7c was synthesized according to general procedure A (1.1 mg, 85%). Final conditions before quenching: peptide—100 μM, 6b—200 μM, 0.1 M Tris (pH 7.5), CH₃CN:H₂O=1:1.

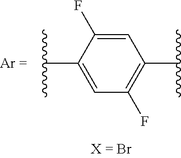 [SMILES: *c1cc(F)c(*)cc1F]

The stapled peptide 7d was synthesized according to general procedure A (1 mg, 70%). Final conditions before quenching: peptide—100 μM, 6c—200 μM, 0.1 M Tris (pH 7.5), CH₃CN:H₂O=1:1.

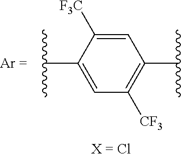 [SMILES: *c1cc(C(F)(F)F)c(*)cc1C(F)(F)F]

The stapled peptide 7e was synthesized according to general procedure A (1.2 mg, 84%). Final conditions before quenching: peptide—100 μM, 6d—200 μM, 0.1 M Tris (pH 7.5), CH₃CN:H₂O=1:1.

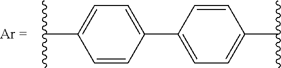 [SMILES: *c1ccc(-c2ccc(*)cc2)cc1]

The stapled peptide 7f was synthesized according to general procedure A (1.1 mg, 92%). Final conditions before quenching: peptide—100 μM, palladium reagent—200 μM, 0.1 M Tris (pH 7.5), CH₃CN:H₂O=1:1.

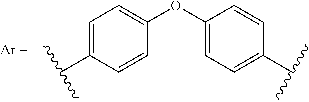 [SMILES: *c1ccc(Oc2ccc(*)cc2)cc1]

The stapled peptide 7g was synthesized according to general procedure A (0.8 mg, 79%). Final conditions before quenching: peptide—100 μM, palladium reagent—200 μM, 0.1 M Tris (pH 7.5), CH₃CN:H₂O=1:1.

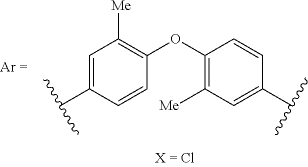 [SMILES: *c1ccc(Oc2ccc(*)cc2C)c(C)c1]

The stapled peptide 7h was synthesized according to general procedure A (1.2 mg, 100%). Final conditions before quenching: peptide—100 μM, 6e—200 μM, 0.1 M Tris (pH 7.5), CH₃CN:H₂O=1:1.

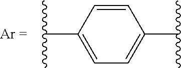 [SMILES: *c1ccc(*)cc1]

The stapled peptide 7i was synthesized according to general procedure B (91%). Final conditions before quenching: peptide—10 μM, 6f—20 μM, 0.1 M Tris (pH 7.5), H₂O.

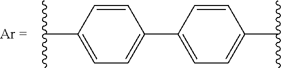 [SMILES: *c1ccc(-c2ccc(*)cc2)cc1]

The stapled peptide 7j was synthesized according to general procedure B (99%). Final conditions before quenching: peptide—10 μM, 6g—20 μM, 0.1 M Tris (pH 7.5), H₂O with 15% acetonitrile for 6g.

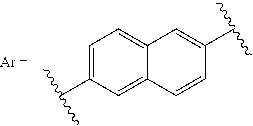 [SMILES: *c1ccc2cc(*)ccc2c1]

The stapled peptide 7k was synthesized according to general procedure B (99%), Final conditions before quenching: peptide—10 μM, 6h—20 μM, 0.1 M Tris (pH 7.5), H₂O with 15% acetonitrile for 6h.

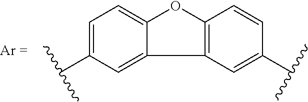 [SMILES: *c1ccc2oc3ccc(*)cc3c2c1]

The stapled peptide 71 was synthesized according to general procedure B (99%), Final conditions before quenching: peptide—10 μM, 6i—20 μM, 0.1 M Tris (pH 7.5), H₂O with 15% acetonitrile for 6i,

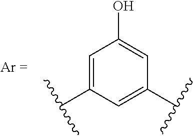 [SMILES: *c1cc(*)cc(O)c1]

The stapled peptide 7m was synthesized according to general procedure B (80%). Final conditions before quenching: peptide—10 μM, 6j—20 μM, 0.1 M Tris (pH 7.5), H₂O.

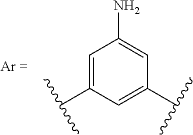 [SMILES: *c1cc(*)cc(N)c1]

The stapled peptide 7n was synthesized according to general procedure B (80%). Final conditions before quenching: peptide—10 μM, 6k—20 μM, 0.1 M Tris (pH 7.5), H₂O.

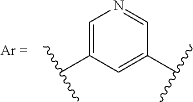 [SMILES: *c1cncc(*)c1]

The stapled peptide 7o was synthesized according to general procedure B (97%). Final conditions before quenching: peptide—10 μM, 61-20 μM, 0.1 M Tris (pH 7.5), H₂O.

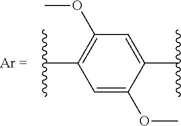 [SMILES: *c1cc(OC)c(*)cc1OC]

The stapled peptide 7p was synthesized according to general procedure B (99%). Final conditions before quenching: peptide—10 μM, 6m—20 μM, 0.1 M Tris (pH 7.5), H₂O.

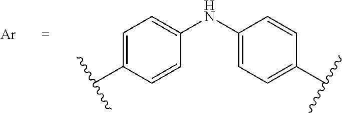

The stapled peptide 7q was synthesized according to general procedure B (99%). Final conditions before quenching: peptide—10 μM, 6n—20 μM, 0.1 M Tris (pH 7.5), H₂O with 15% acetonitrile for 6n.

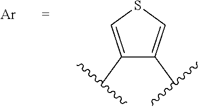

The stapled peptide 7r was synthesized according to general procedure B (99%). Final conditions before quenching: peptide—10 μM, 6o—20 μM, 0.1 M Tris (pH 7.5), H₂O with 15% acetonitrile for 60.

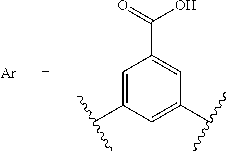

The stapled peptide 7s was synthesized according to general procedure B (100%). Final conditions before quenching: peptide—10 μM, 6p—20 μM, 0.1 M Tris (pH 7.5). H₂O.

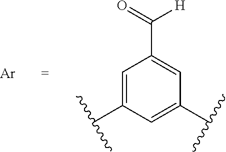

The stapled peptide 7t was synthesized according to general procedure B (99%). Final conditions before quenching: peptide—10 μM, 6q—20 μM, 0.1 M Tris (pH 7.5), H₂O.

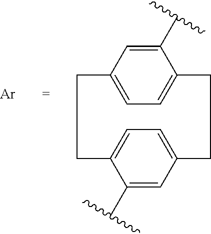

The stapled peptide 7u was synthesized according to general procedure B (99%). Final conditions before quenching: peptide—10 μM, 6r—20 μM, 0.1 M Tris (pH 7.5), H₂O with 15% acetonitrile for 6r.

### Example 7—Protein Experiments

### Protein Expression and Purification

pET-SUMO-DARPin plasmids were constructed as described in Liao, X., Rabideau, A. E. & Pentelute, B. L. Delivery of antibody mimics into mammalian cells via anthrax toxin protective antigen. Chembiochem 15, 2458-2466 □(2014). Cysteine mutations were introduced by site-directed mutagenesis using QuickChange Lightning Single Site-directed Mutagenesis Kit (Agilent) following manufacturer's instructions. Sequences of generated protein constructs are summarized in Table S6.

E. coli BL21(DE3) cells transformed with pET-SUMO-Protein plasmid were grown in 1 L of LB medium containing kanamycin (30 μg/mL) at 37° C. until OD600=0.6. Then, expression was induced by the addition of 0.5 mM IPTG overnight at 30° C. After harvesting the cells by centrifugation (6,000 rpm for 10 min), the cell pellet was lysed by sonication in 25 mL of 50 mM Tris and 150 mM NaCl (pH 7.5) buffer containing 15 mg lysozyme (Calbiochem), 1 mg DNase 1 (Sigma-Aldrich), and 0.5 tablet of protease inhibitor cocktail (Roche Diagnostics, Germany). The resulting suspension was centrifuged at 17,000 rpm for 30 min to remove cell debris. The supernatant was loaded onto a 5 mL HisTrap FF crude Ni-NTA column (GE Healthcare, UK), first washed with 40 mL of 20 mM Tris and 150 mM NaCl (pH 8.5), and then washed with 40 mL of 40 mM imidazole in 20 mM Tris and 150 mM NaCl (pH 8.5). The protein was eluted from the column with buffer containing 500 mM imidazole in 20 mM Tris and 150 mM NaCl (pH 8.5), Imidazole was removed from protein using a HiPrep 26/10 Desalting column (GE Healthcare, UK), the protein was eluted into 20 mM Tris and 150 mM NaCl (pH 7.5) buffer. The protein was analyzed by LC-MS to confirm its purity and molecular weight.

SUMO group on SUMO-Protein was cleaved by incubating 1 μg of SUMO protease per mg of protein at room temperature for 60 min. The crude reaction mixture was loaded onto a 5 ml, HisTrap FF erode Ni-NTA column (GE Healthcare, UK) and the flow through containing the desired protein was collected. The protein was analyzed by LC-MS confirming sample purity and molecular weight. Purified proteins were concentrated using Amicon 3K concentrator (50 mL, EMD Millipore); protein aliquots were flash frozen and stored in −80° C. freezer.

### Protein Labeling Experiments

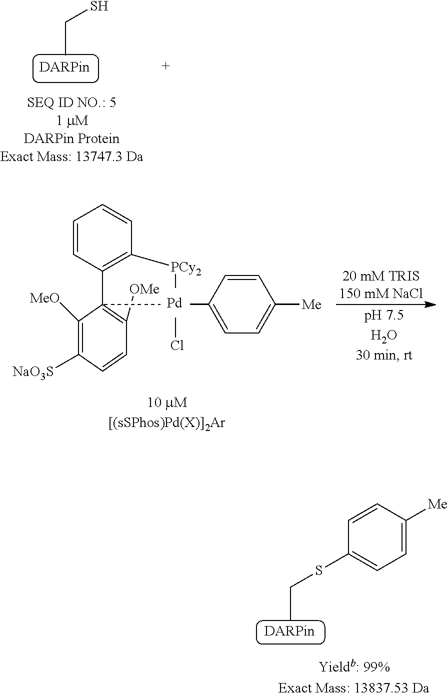

To a solution of protein (500 pmoles) in 475 μL of 20 mM Tris and 150 mM NaCl buffer (pH 7.5) was added palladium-tolyl complex OA-3 (25 μL, 200 μM) in water. The solution was pipetted up and down 10 times to ensure proper reagent mixing. The reaction mixture was left at room temperature for 30 min. After this time, the reaction was quenched by the addition of 3-thiopropionic acid (25 μL, 2 mM) dissolved in 20 mM Tris and 150 mM NaCl buffer (pH 7.5). After an additional 5 min at rt, 500 μL of 1:1 CH₃CN/H2O (v/v) containing 0.2% TFA was added and the resulting mixture was analyzed by LC-MS.

TABLE 2
DARPin-Cys protein sequence and calculated mass

| | Calculated |
|---|---|
| Sequence | mass |
| GGCGGSDLGKKLLEAARAGQDDEVRILMANGADVNAYD | 13747.3 Da |
| DNGVTPLHLAAFLGHLEIVEVLLKYGADVNAADSWGTT | |
| PLHLAATWGHLEIVEVLLKHGADVNAQDKFGKTAF | |
| DISIDNGNEDLAEILQKLN□ (SEQ ID NO: 5) | |

### Example 8—ICP MS Analysis

Two distinct macrocyclic peptides, chosen at random (P2-10 and P2-14), were dissolved in 0.4 mL of concentrated nitric acid. This solution was sonicated and diluted with MilliQ pure water to 0.2% nitric acid concentration. ICP-MS was performed on the resulting mixtures. Calibration curves were generated using Pd ICP-MS standards for a range between 1000 ppm and 100 ppb. There was no palladium found to be remaining in the peptide after purification. Initial palladium content was ˜300 ppm used to perform the macrocyclization reactions indicating over 99% palladium removal.

### INCORPORATION BY REFERENCE

AH of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

1. A method of functionalizing a thiol or a selenol in a biopolymer, comprising:
contacting a biopolymer comprising a thiol or selenol moiety with a reagent of structural formula II, thereby generating a functionalized biopolymer, wherein the thiol or selenol moiety has been transformed to —S—Ar¹ or —Se—Ar¹: 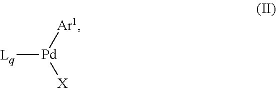
wherein
Ar¹ is selected from the group consisting of Ar¹ is selected from the group consisting of 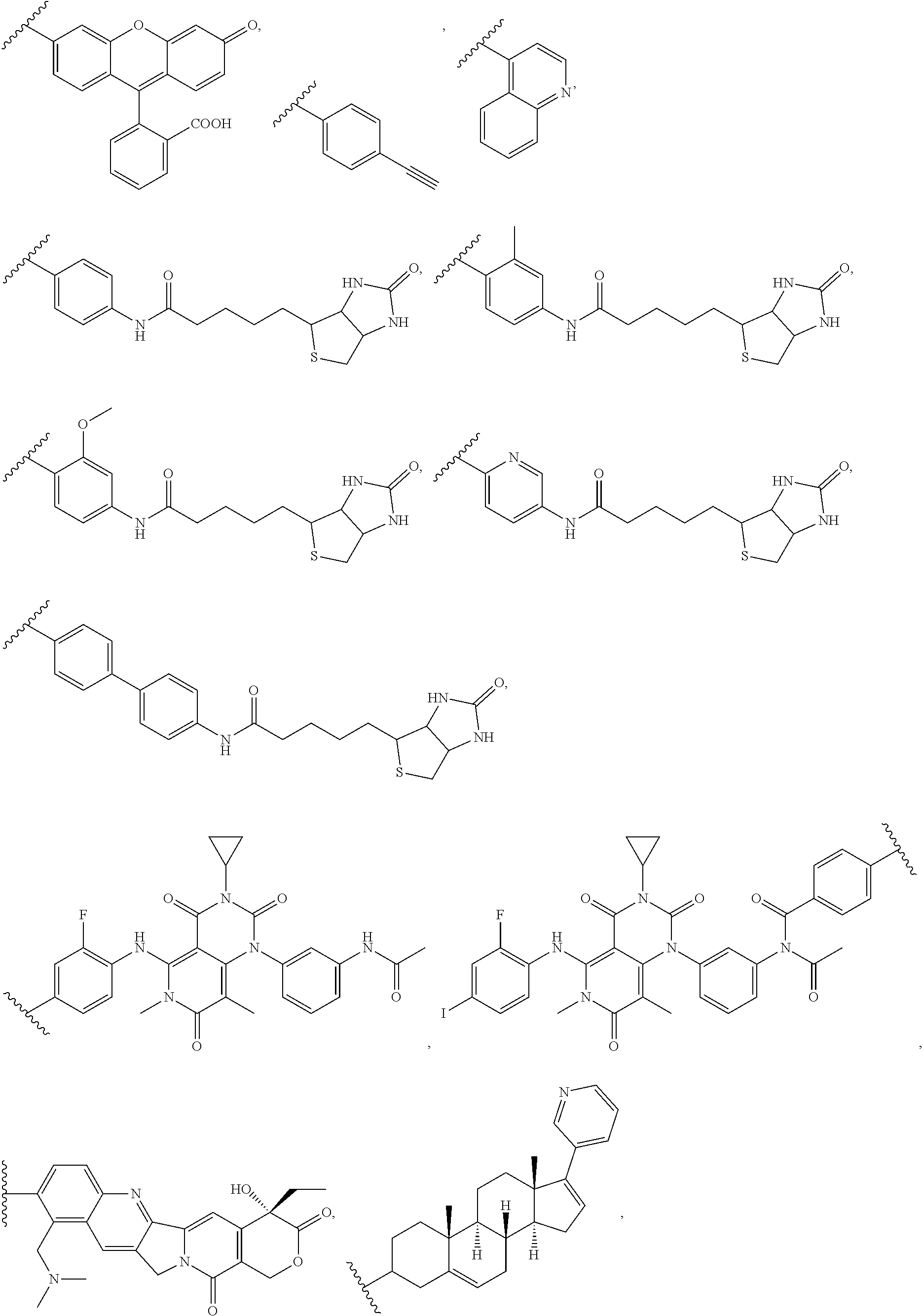 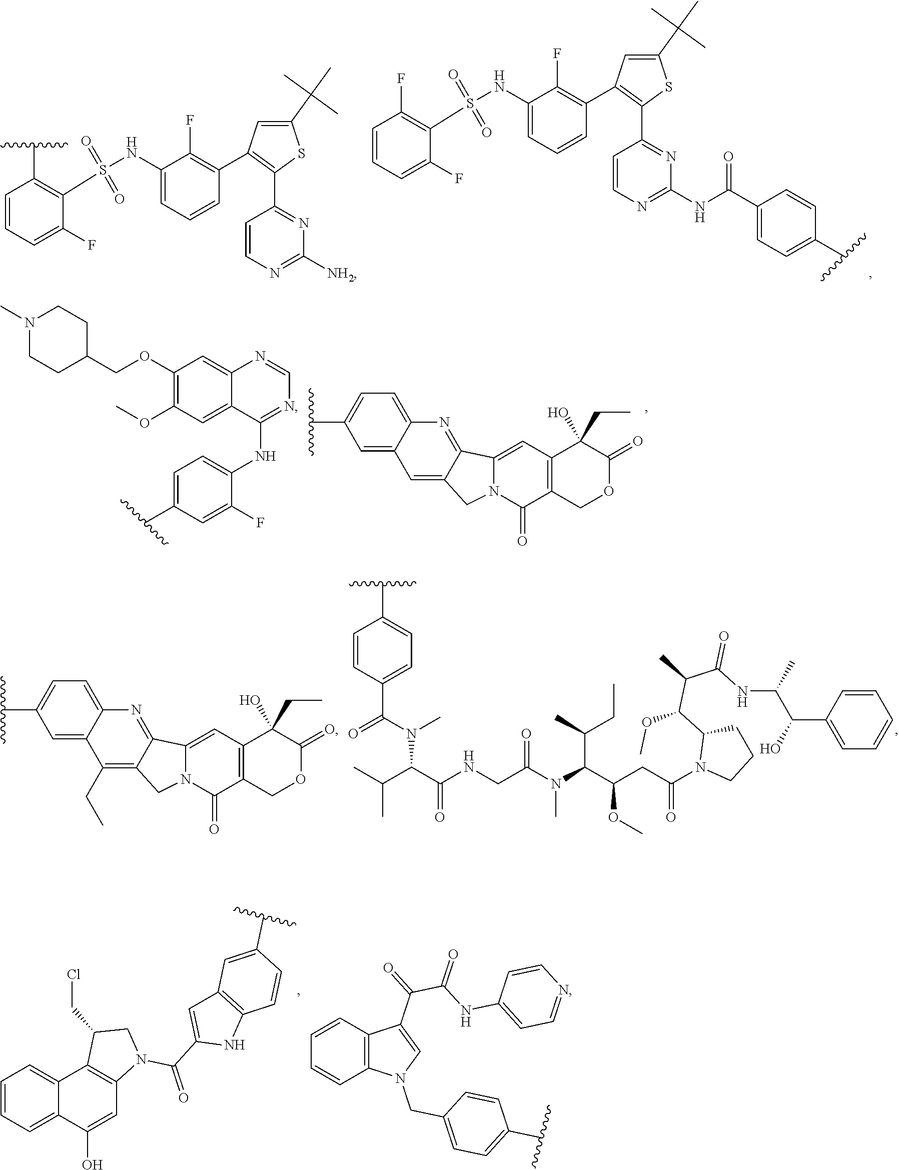 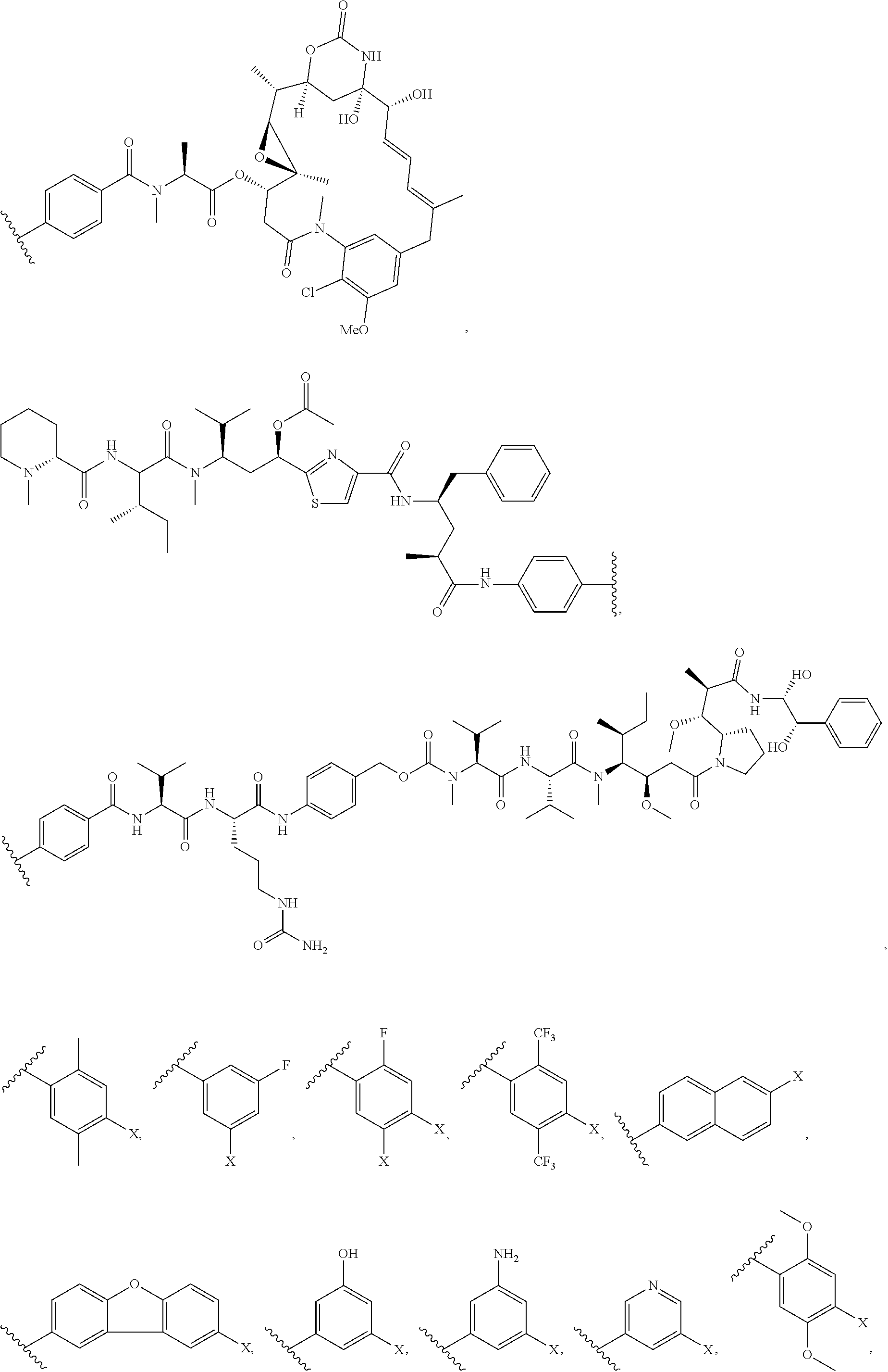 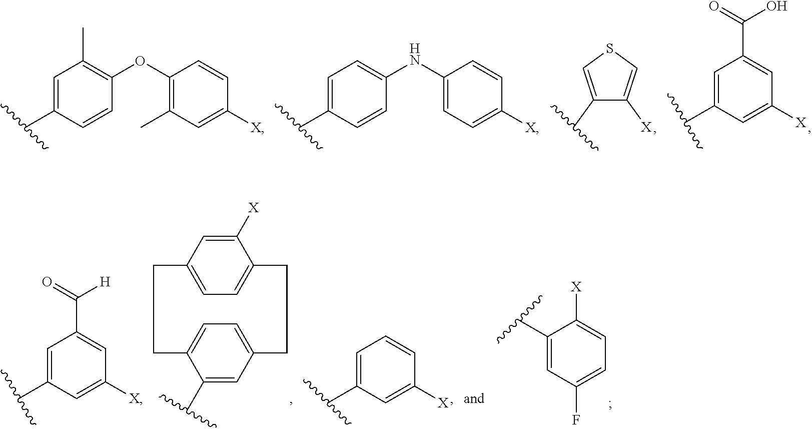
X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;
L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine; and
q is 1 or 2.

2. The method of claim 1, wherein the biopolymer is a peptide, an oligopeptide, a polypeptide, a protein, an antibody, an antibody fragment, an oligonucleotide, a polynucleotide, an oligosaccharide, or a polysaccharide.

3. (canceled)

4. A method of functionalizing a thiol or a selenol, wherein said method is represented by Scheme 1: 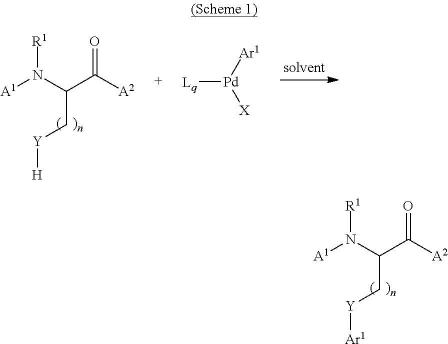
wherein:
A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;
A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;
Y is S or Se;
R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;
Ar¹ is selected from the group consisting of 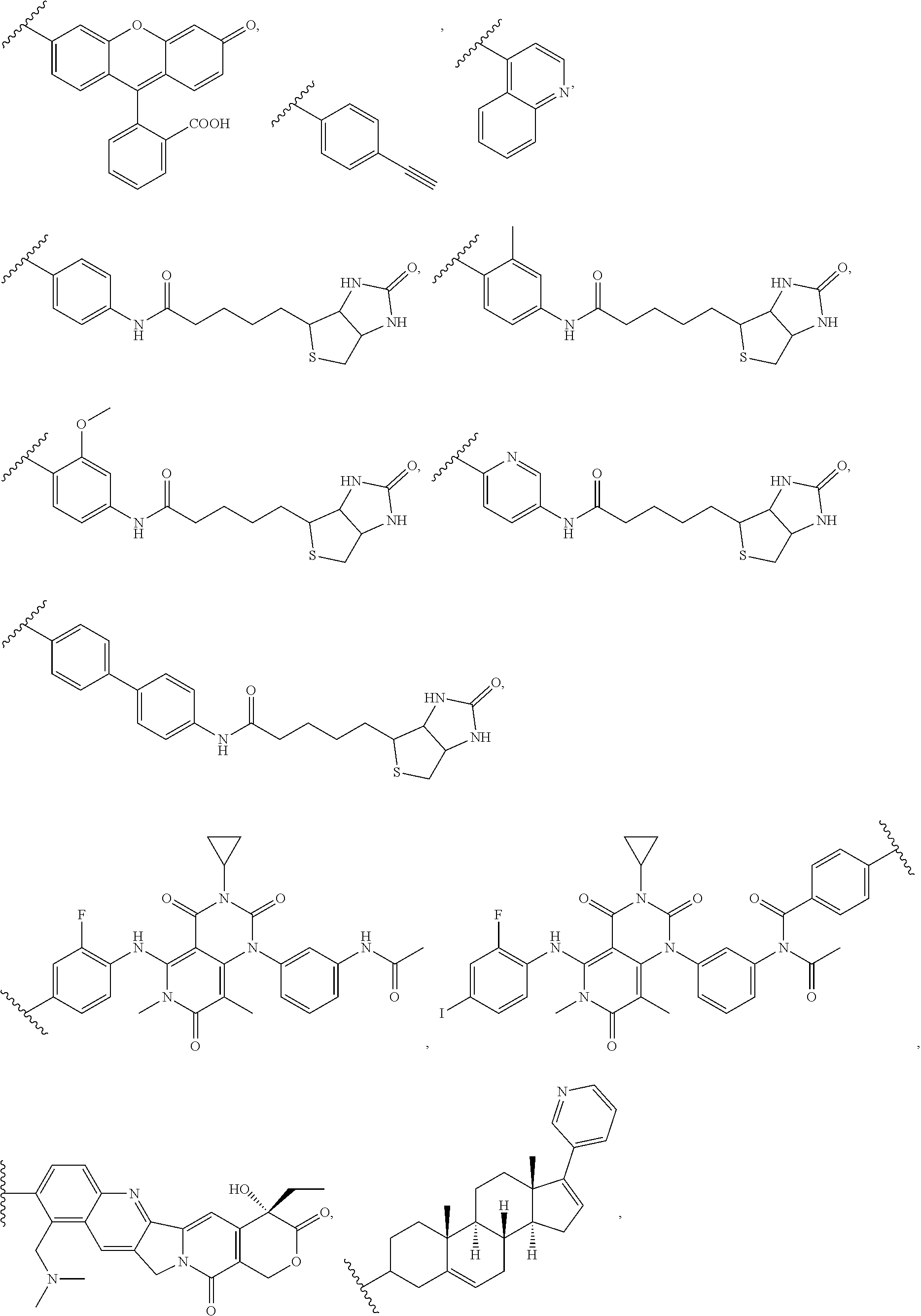 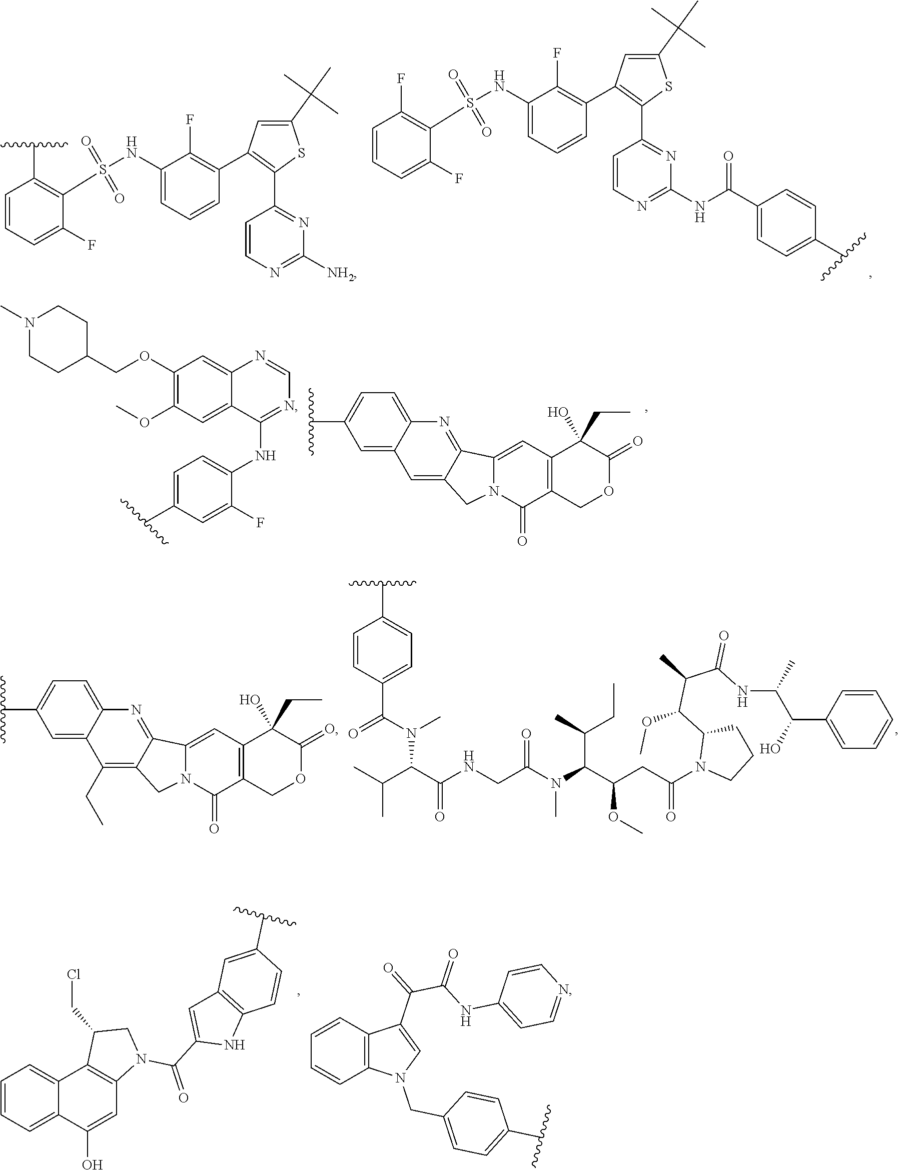 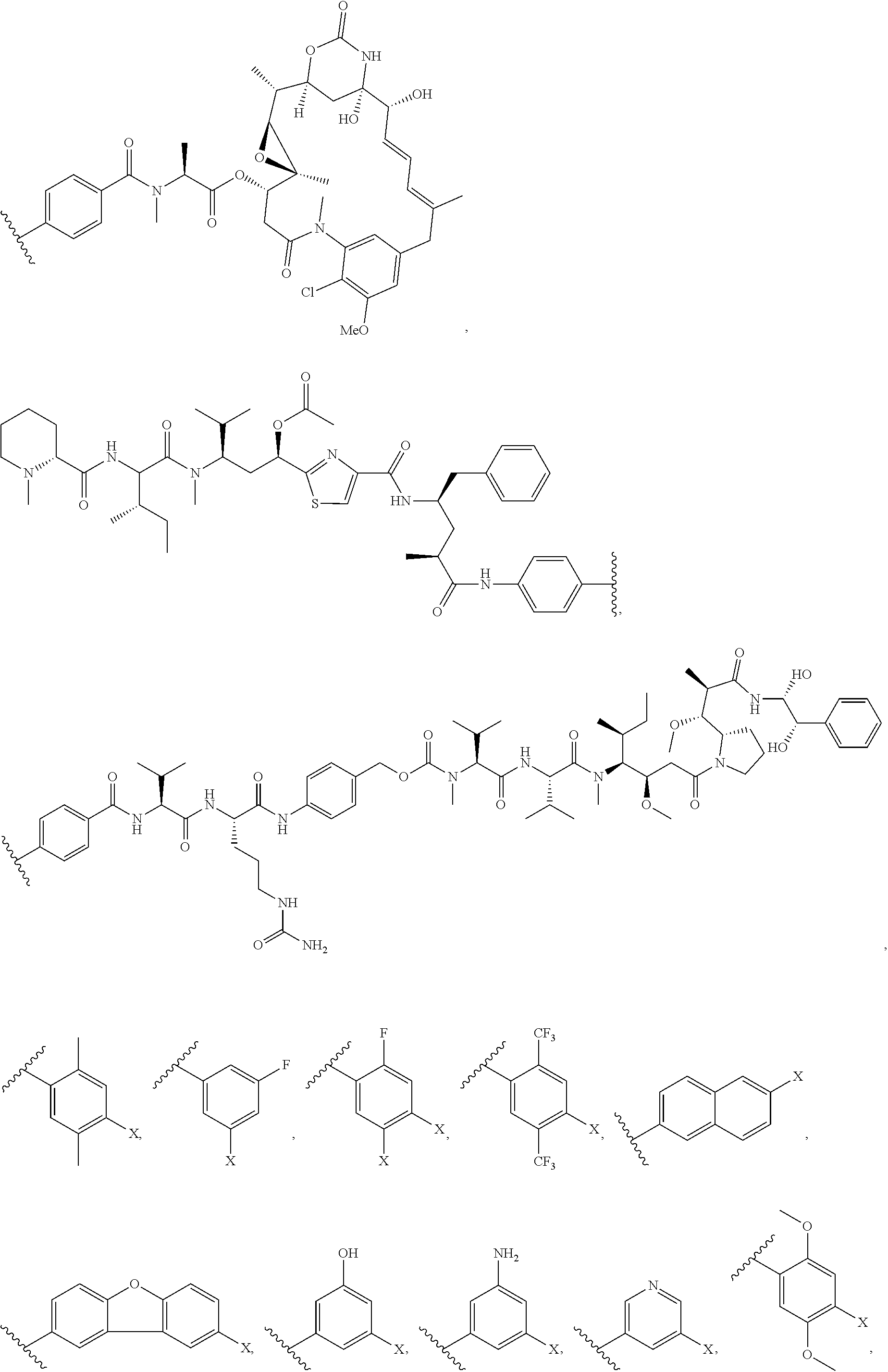 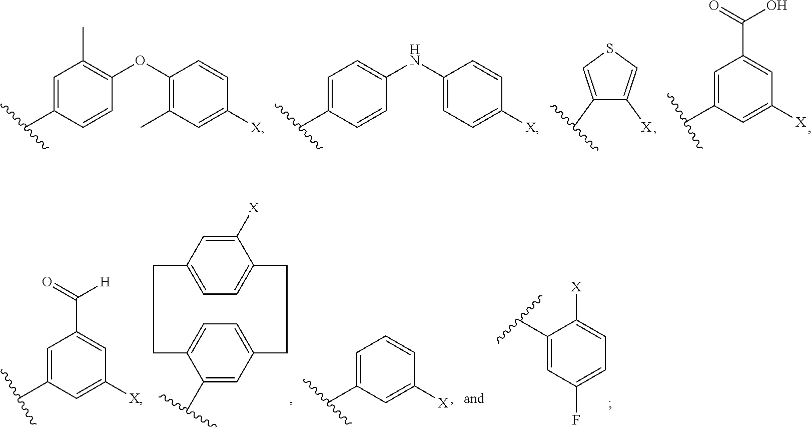
X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;
L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;
n is an integer from 1-5;
q is 1 or 2; and
solvent comprises water, and a polar protic solvent, a polar aprotic solvent, or a non-polar solvent.

5. (canceled)

6. The method of claim 1, wherein L is selected from the group consisting of 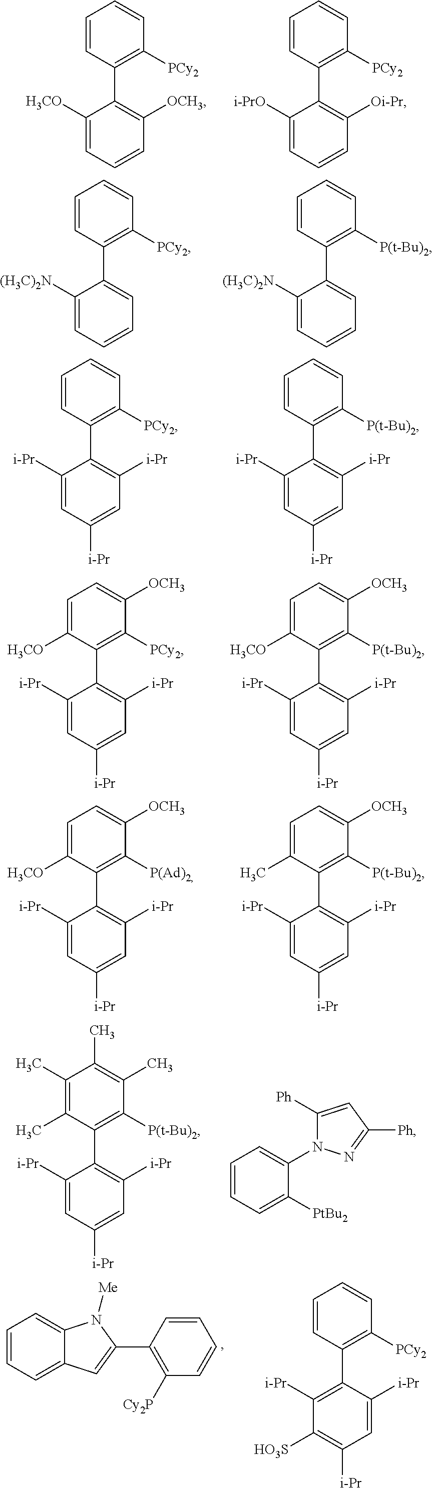
or a salt thereof, 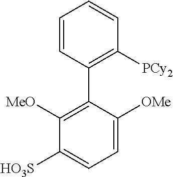
or a salt thereof, 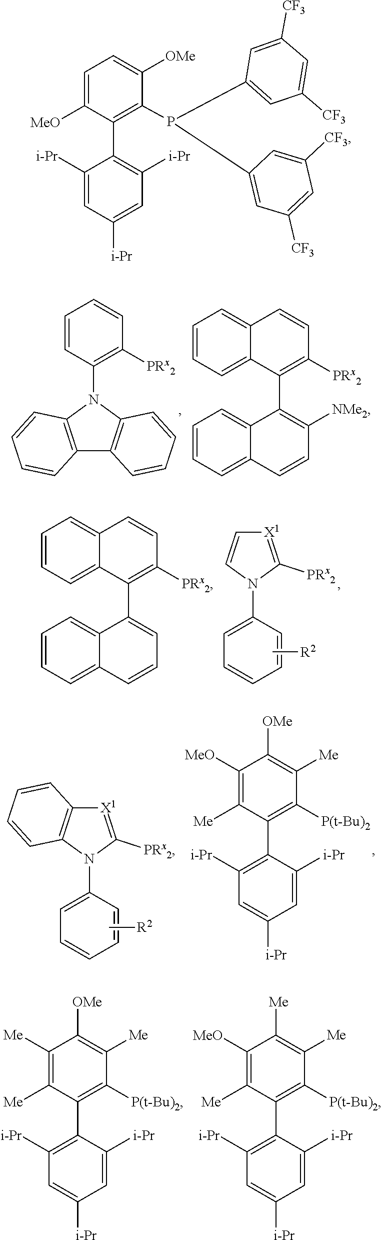 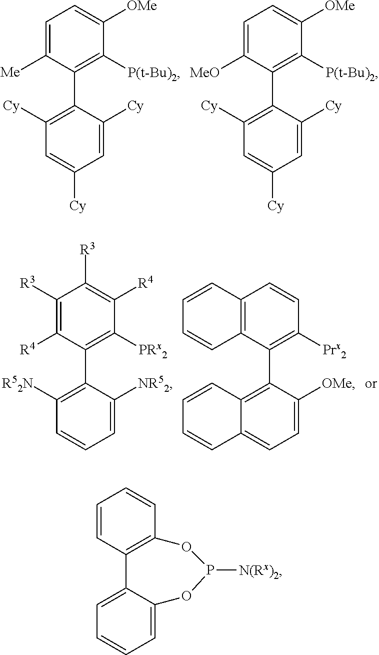
wherein
R^{x} is independently for each occurrence alkyl, aralkyl, cycloalkyl, or aryl;
X¹ is CH or N;
R² is H or alkyl;
R³ is H or alkyl;
R⁴ is H, alkoxy, or alkyl; and
R⁵ is alkyl or aryl.

7-13. (canceled)

14. The method of claim 4, wherein the limiting reagent is

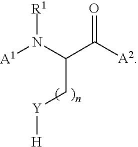

15-20. (canceled)

21. The method of claim 4, wherein the solvent comprises an aqueous buffer.

22. The method of claim 4, further comprising contacting compound III, 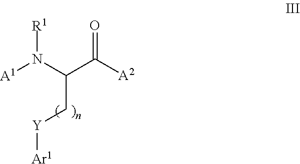
with a compound containing a thiol moiety or a selenol moiety; thereby yielding a coupling product.

23. (canceled)

24. The method of claim 22, wherein the compound containing a thiol moiety or a selenol moiety is a biomolecule selected from the group consisting of a natural or unnatural amino acid, a plurality of natural or unnatural amino acids, a peptide, an oligopeptide, a polypeptide, and a protein.

25-44. (canceled)

45. A method of functionalizing a thiol or a selenol, wherein said method is represented by Scheme 1: 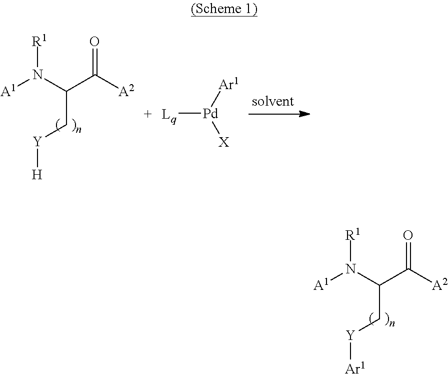
wherein:
A¹ is H, an amine protecting group, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;
A² is NH₂, NH(amide protecting group), N(amide protecting group), OH, O(carboxylate protecting group), a natural or unnatural amino acid, a plurality of natural amino acids or unnatural amino acids, a peptide, an oligopeptide, a polypeptide, a protein, an antibody, or an antibody fragment;
Y is S or Se;
R¹ is H, alkyl, arylalkyl, acyl, aryl, alkoxycarbonyl, aryloxycarbonyl, a natural or unnatural amino acid, a plurality of natural amino acids or unnatural amino acids, a peptide, an oligopeptide, a polypeptide, a protein, an antibody, or an antibody fragment;
Ar¹ is optionally substituted aryl, heteroaryl, alkenyl, or cycloalkenyl;
X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;
L is independently for each occurrence a trialkylphosphine, a triarylphosphine, a dialkylarylphosphine, an alkyldiarylphosphine, an (alkenyl)(alkyl)(aryl)phosphine, an alkenyldiarylphosphine, an alkenyldialkylphosphine, a phosphine oxide, a bis(phosphine), a phosphoramide, a triarylphosphonate, an N-heterocyclic carbene, an optionally substituted phenanthroline, an optionally substituted iminopyridine, an optionally substituted 2,2′-bipyridine, an optionally substituted diimine, an optionally substituted triazolylpyridine, or an optionally substituted pyrazolyl pyridine;
n is an integer from 1-5;
q is 1 or 2; and
solvent is water.

46-47. (canceled)

48. The method of claim 45, wherein L is independently for each occurrence
represented by structure L: 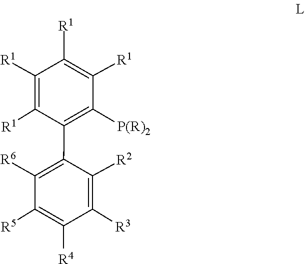
wherein
R is selected independently for each occurrence from the group consisting of alkyl, cycloalkyl, aryl, heteroaryl, aralkyl, heteroaralkyl, and —(CH₂)ₘ—R⁸⁰;
R¹ is selected independently for each occurrence from the group consisting of hydrogen, halogen, alkyl, cycloalkyl, heterocycloalkyl, aryl, heteroaryl, aralkyl, heteroaralkyl, —OR⁸, —N(R⁸)-₂, —Si(R⁸)₃, and —(CH₂)ₘ—R⁸⁰;
R² is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;
R³ is selected from the group consisting of halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;
R⁴ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;
R⁵ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;
R⁶ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;
R⁷ is selected independently for each occurrence from the group consisting of —C(O)OM, —C(O)SM, —C(S)SM, —C(NR⁸)OM, —C(NR⁸)SM, —S(O)OM, —S(O)SM, —S(O)₂OM, —S(O)₂SM, —P(O)(OM)₂, —P(O)(OR⁸)OM, —P(O)(OR⁸)NR⁸M, —P(O)(OR⁸)SM, —N(R⁸)₃M, —P(R⁸)₃M, —P(OR⁸)₃M and —N(R⁸)C(NR⁸R⁸)NR⁸R⁸M;
R⁸ is selected independently for each occurrence from the group consisting of hydrogen, alkyl, cycloalkyl, heterocycloalkyl, aryl, heteroaryl, aralkyl, and heteroaralkyl;
M is an alkali metal or an alkali earth metal;
R⁸⁰ represents an unsubstituted or substituted aryl, a cycloalkyl, a cycloalkenyl, a heterocycle, or a polycycle;
m is independently for each occurrence an integer in the range 0 to 8 inclusive;
provided that at least one of R³, R⁴ or R⁵ is R⁷; and
the ligand is achiral or, when chiral, is a single stereoisomer or a mixture of stereoisomers.

49-60. (canceled)

61. The method of claim 45, wherein Ar¹ is (C₆-C₁₀)carbocyclic aryl, (C₃-C₁₂)heteroaryl, (C₃-C₁₄)polycyclic aryl, or alkenyl; and Ar¹ is optionally substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, and —(CH₂)_{P}—FG-R⁷;
p is independently for each occurrence an integer from 0-10;
FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);
R⁷ is independently for each occurrence selected from the group consisting of H, alkyl, cycloalkyl, aryl, aralkyl, alkenyl, and alkynyl; and
if two or more substituents are present on Ar¹, then two of said substituents taken together may form a ring;
wherein at least one of the one or more substituents is halide.

62. The method of claim 45, wherein Ar¹ is covalently linked to a fluorophore, an imaging agent, a detection agent, a biomolecule, a therapeutic agent, a lipophilic moiety, a member of a high-affinity binding pair, or a cell-receptor targeting agent.

63-68. (canceled)

69. The method of claim 45, wherein Ar¹ is selected from the group consisting of

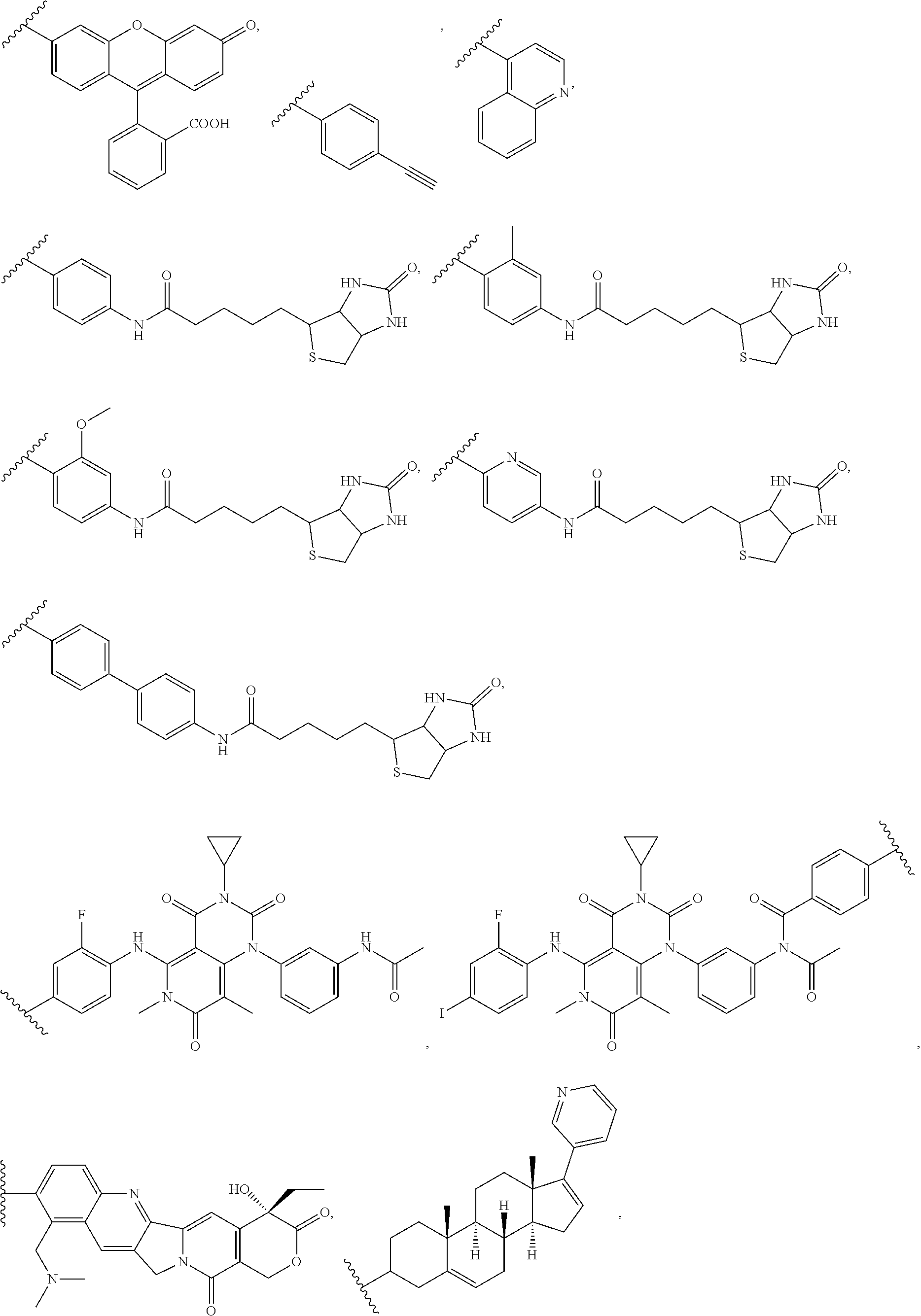

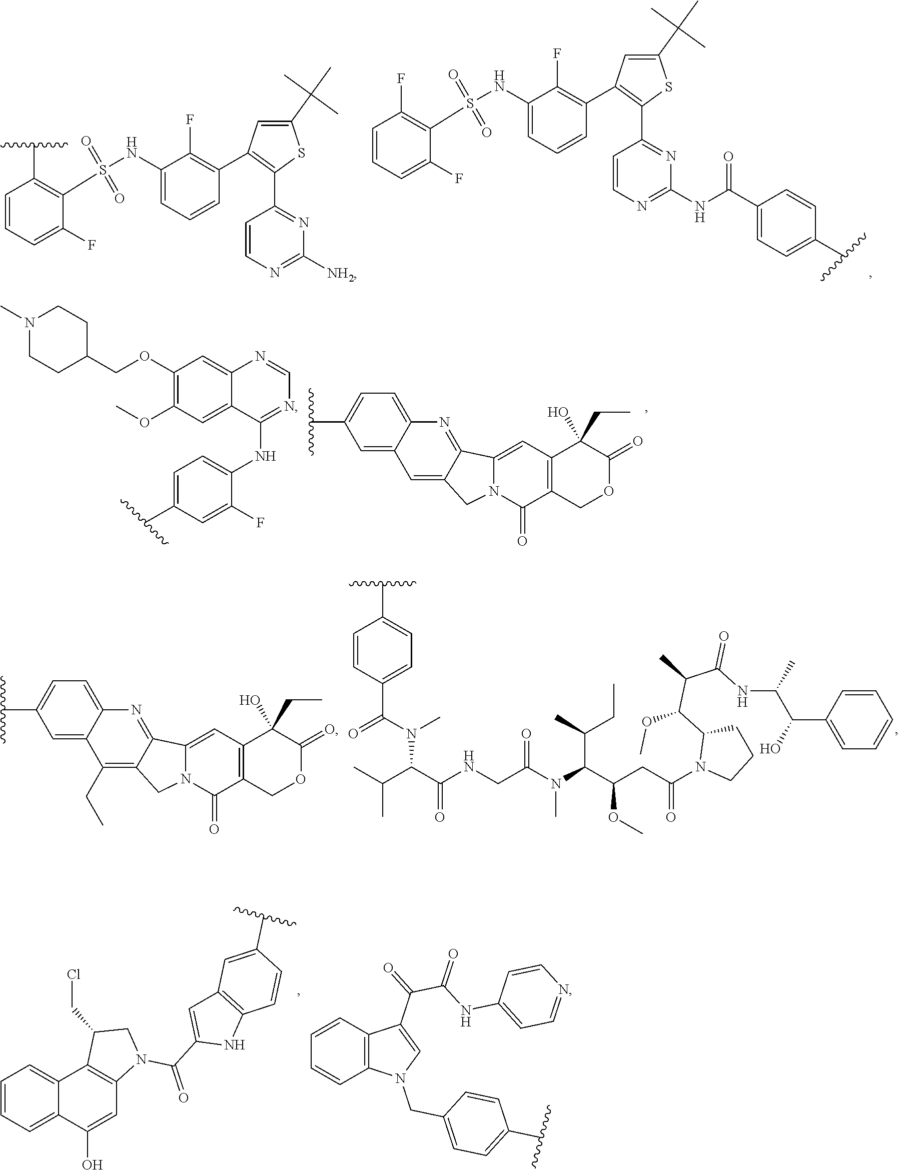

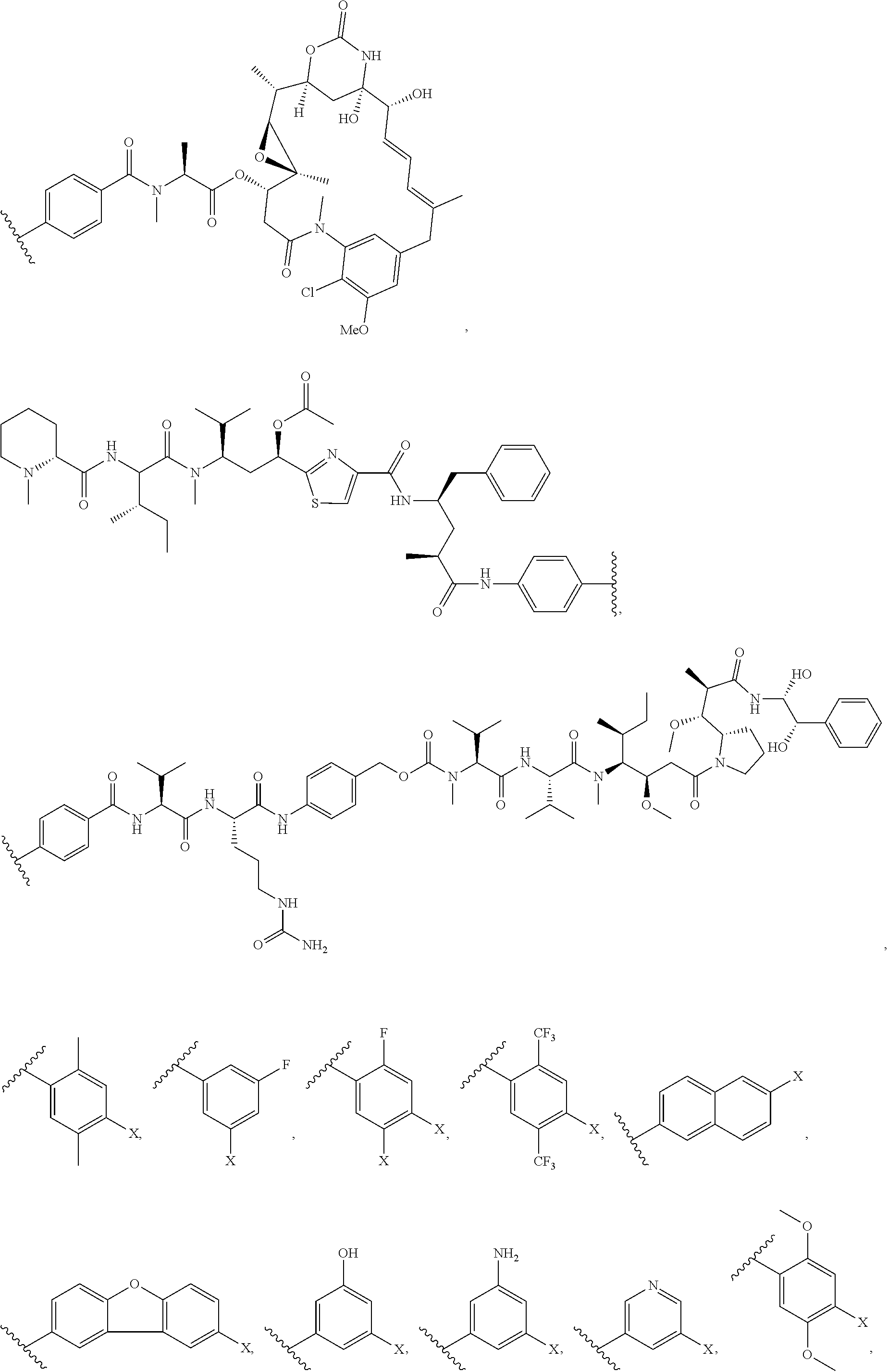

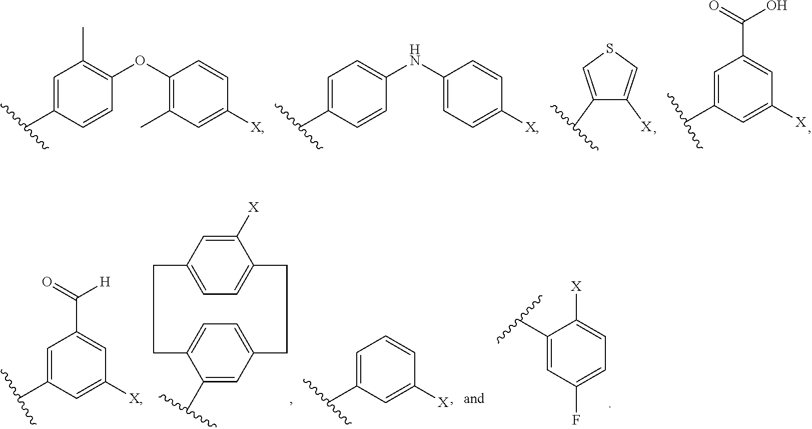

70. (canceled)

71. The method of claim 45, wherein A¹ and A² are independently a natural or unnatural amino acid, a plurality of natural or unnatural amino acids, a peptide, an oligopeptide, a polypeptide, or a protein.

72-74. (canceled)

75. The method of claim 45, wherein the limiting reagent is

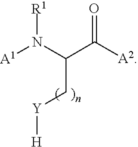

76-115. (canceled)

116. A method of preparing a palladium complex, wherein said method is represented by Scheme 4: 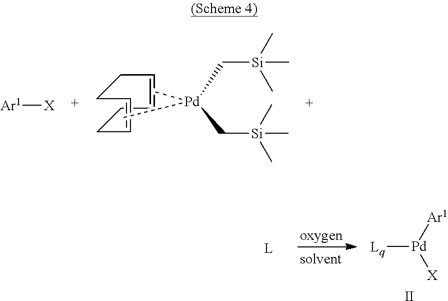
Ar¹ is optionally substituted aryl, heteroaryl, alkenyl, or cycloalkenyl;
X is a halide, triflate, tetrafluoroborate, tetraarylborate, hexafluoroantimonate, bis(alkylsulfonyl)amide, tetrafluorophosphate, hexafluorophosphate, alkylsulfonate, haloalkylsulfonate, arylsulfonate, perchlorate, bis(fluoroalkylsulfonyl)amide, bis(arylsulfonyl)amide, (fluoroalkylsulfonyl)(fluoroalkyl-carbonyl)amide, nitrate, nitrite, sulfate, hydrogensulfate, alkyl sulfate, aryl sulfate, carbonate, bicarbonate, carboxylate, phosphate, hydrogen phosphate, dihydrogen phosphate, phosphinate, or hypochlorite;
L is independently for each occurrence represented by structure L: 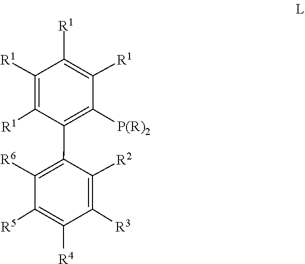
wherein
R is selected independently for each occurrence from the group consisting of alkyl, cycloalkyl, aryl, heteroaryl, aralkyl, heteroaralkyl, and —(CH₂)ₘ—R⁸⁰;
R¹ is selected independently for each occurrence from the group consisting of hydrogen, halogen, alkyl, cycloalkyl, heterocycloalkyl, aryl, heteroaryl, aralkyl, heteroaralkyl, —OR⁸, —N(R⁸)-₂, —Si(R⁸)₃, and —(CH₂)ₘ—R⁸⁰;
R² is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;
R³ is selected from the group consisting of halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;
R⁴ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;
R⁵ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, —R⁷, and —(CH₂)ₘ—R⁸⁰;
R⁶ is selected from the group consisting of hydrogen, halogen, alkyl, cycloalkyl, heterocycloalkyl, aryl, heteroaryl, aralkyl, heteroaralkyl, —OR⁸, —N(R⁸)₂, —Si(R⁸)₃, and —(CH₂)ₘ—R⁸⁰;
R⁷ is selected independently for each occurrence from the group consisting of —C(O)OM, —C(O)SM, —C(S)SM, —C(NR⁸)OM, —C(NR⁸)SM, —S(O)OM, —S(O)SM, —S(O)₂OM, —S(O)₂SM, —P(O)(OM)₂, —P(O)(OR⁸)OM, —P(O)(OR⁸)NR⁸M, —P(O)(OR⁸)SM, —N(R⁸)₃M, —P(R⁸)₃M, —P(OR⁸)₃M and —N(R⁸)C(NR⁸R⁸)NR⁸R⁸M;
R⁸ is selected independently for each occurrence from the group consisting of hydrogen, alkyl, cycloalkyl, heterocycloalkyl, aryl, heteroaryl, aralkyl, and heteroaralkyl;
M is an alkali metal or an alkali earth metal;
R⁸⁰ represents an unsubstituted or substituted aryl, a cycloalkyl, a cycloalkenyl, a heterocycle, or a polycycle;
m is independently for each occurrence an integer in the range 0 to 8 inclusive;
provided that at least one of R³, R⁴ or R⁵ is R⁷;
the ligand is achiral or, when chiral, is a single stereoisomer or a mixture of stereoisomers;
q is 1 or 2; and
solvent is a polar protic solvent, a polar aprotic solvent, or a non-polar solvent;
wherein the method occurs in the presence of oxygen.

117-125. (canceled)

126. The method of claim 116, wherein L is selected from the group consisting of 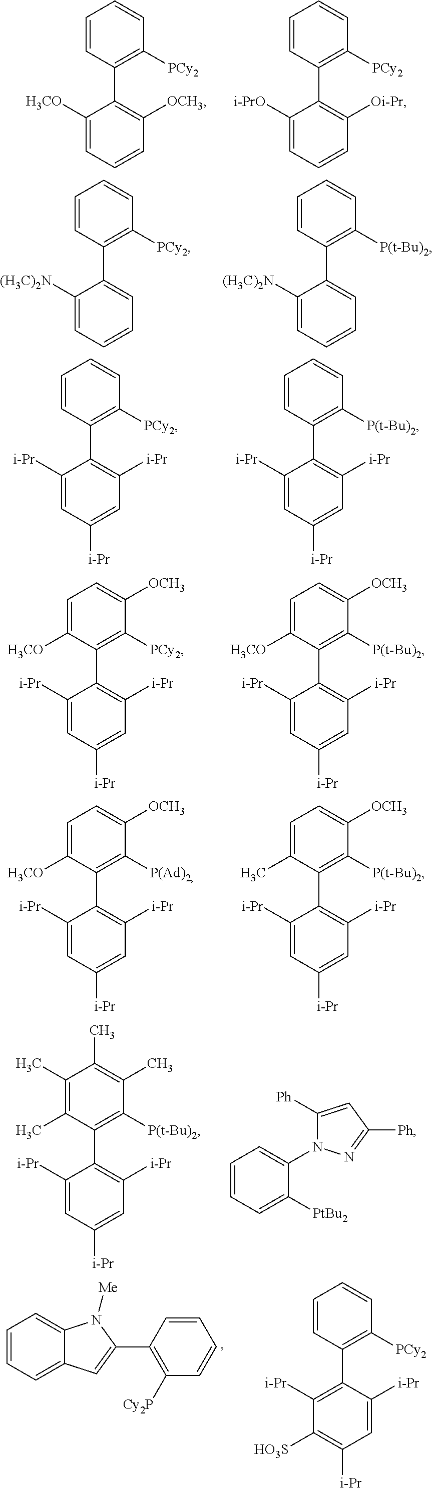
salt thereof, 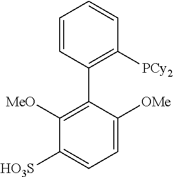
or a salt thereof, 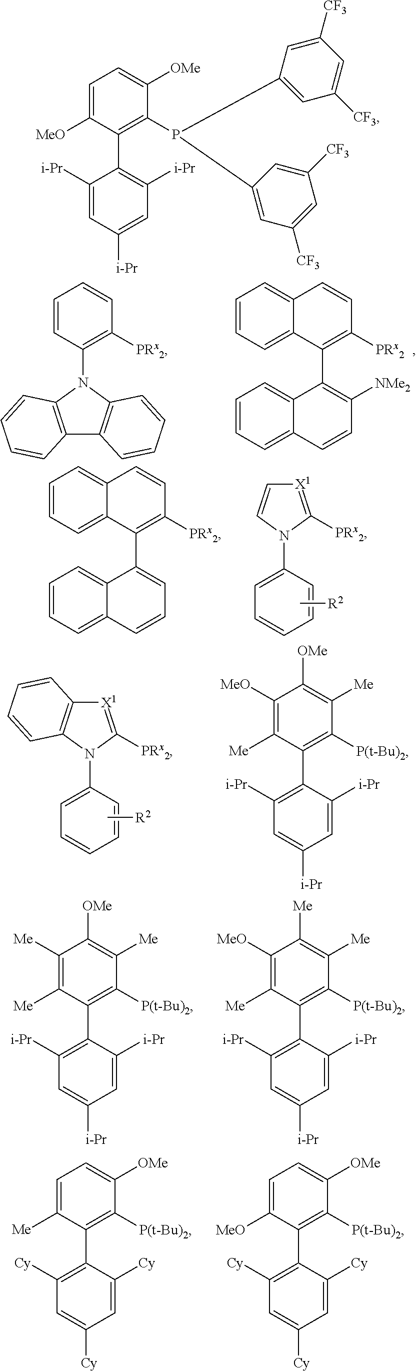 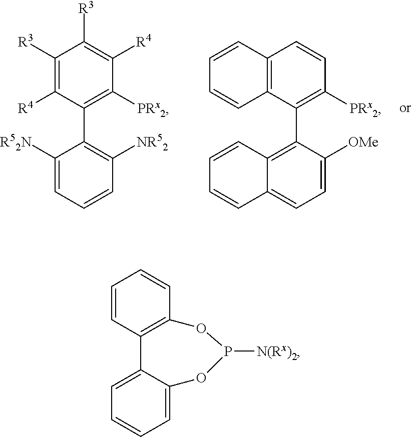
wherein
R^{x} is independently for each occurrence alkyl, aralkyl, cycloalkyl, or aryl;
X¹ is CH or N;
R² is H or alkyl;
R³ is H or alkyl;
R⁴ is H, alkoxy, or alkyl; and
R⁵ is alkyl or aryl.

127-129. (canceled)

130. The method of claim 116, wherein Ar¹ is (C₆-C₁₀)carbocyclic aryl, (C₃-C₁₂)heteroaryl, (C₃-C₁₄)polycyclic aryl, or alkenyl; and Ar¹ is optionally substituted by one or more substituents independently selected from the group consisting of halide, acyl, azide, isothiocyanate, alkyl, aralkyl, alkenyl, alkynyl or protected alkynyl, alkoxyl, arylcarbonyl, cycloalkyl, formyl, haloalkyl, hydroxyl, amino, nitro, sulfhydryl, amido, phosphonate, phosphinate, alkylthio, sulfonyl, sulfonamido, heterocyclyl, aryl, heteroaryl, —CF₃, —CF₂R⁷, —CFR⁷₂, —CN, polyethylene glycol, polyethylene imine, and —(CH₂)_{P}—FG-R⁷;
p is independently for each occurrence an integer from 0-10;
FG is independently for each occurrence selected from the group consisting of C(O), CO₂, O(CO), C(O)NR⁷, NR⁷C(O), O, Si(R⁷)₂, C(NR⁷), (R⁷)₂N(CO)N(R⁷)₂, OC(O)NR⁷, NR⁷C(O)O, and C(N═N);
R⁷ is independently for each occurrence selected from the group consisting of H, alkyl, cycloalkyl, aryl, aralkyl, alkenyl, and alkynyl; and
if two or more substituents are present on Ar¹, then two of said substituents taken together may form a ring;
wherein at least one of the one or more substituents is halide.

131-137. (canceled)

138. The method of claim 116, wherein Ar¹ is selected from the group consisting of

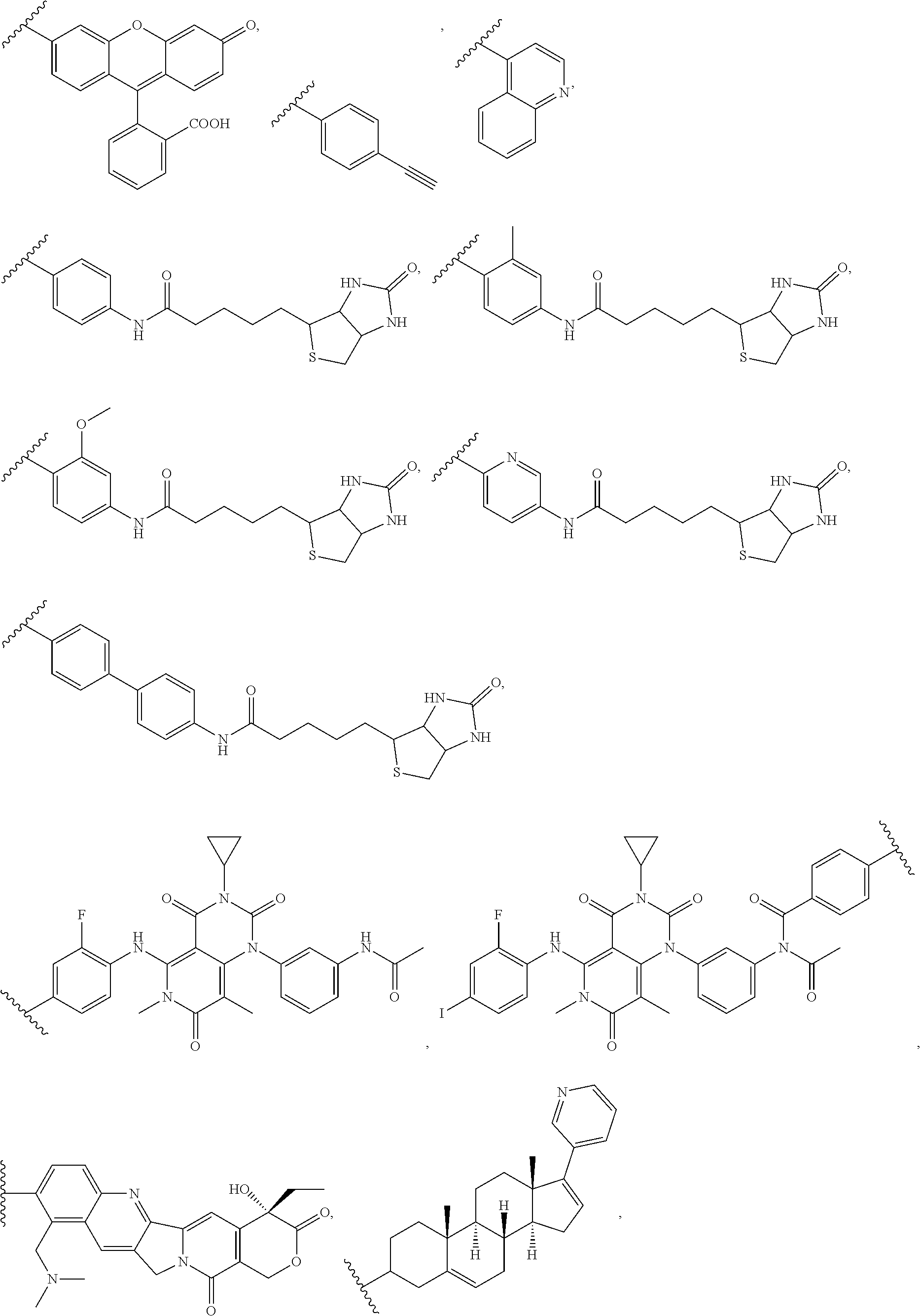

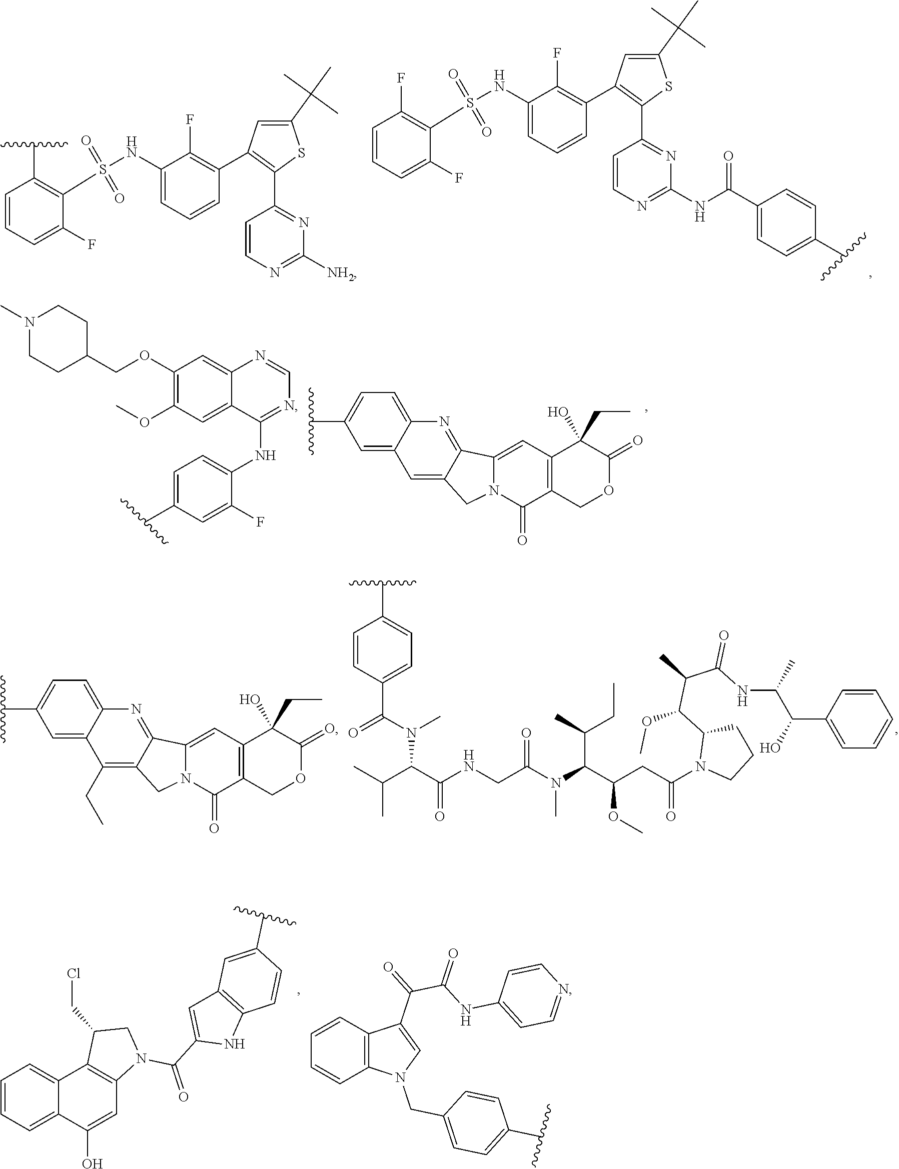

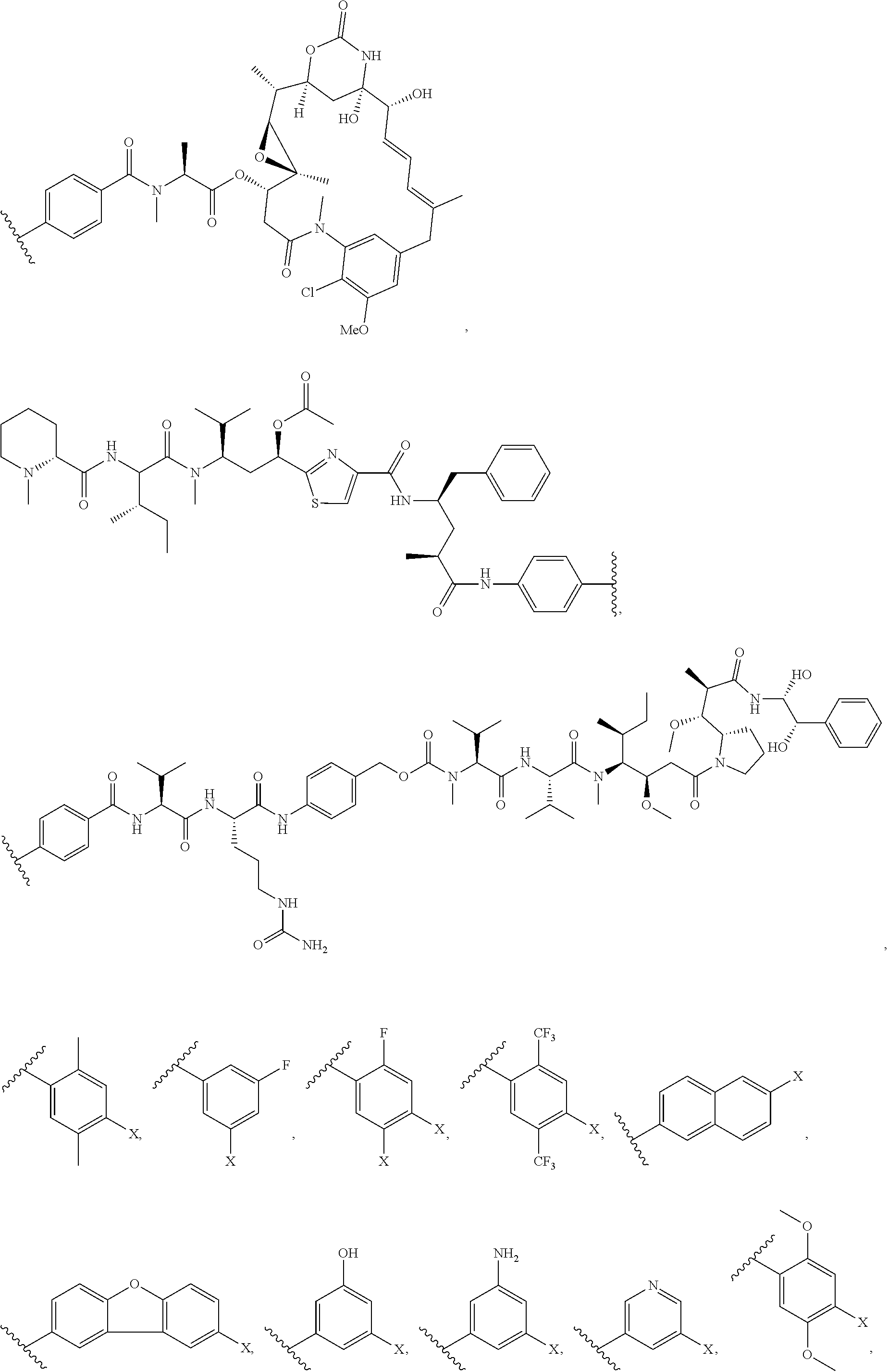

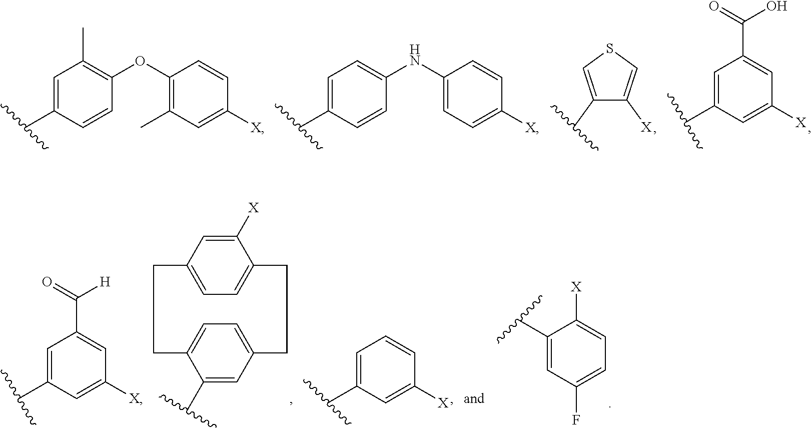

139-143. (canceled)

144. The method of claim 4, wherein L is selected from the group consisting of 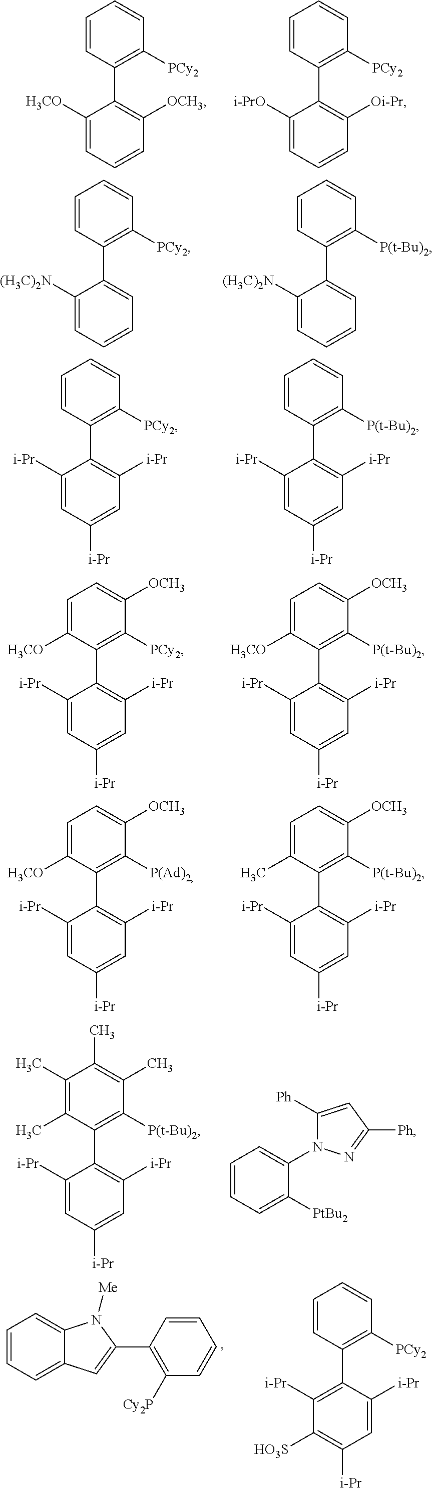
or a salt thereof, 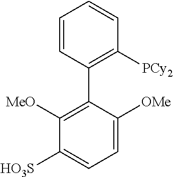
or a salt thereof, 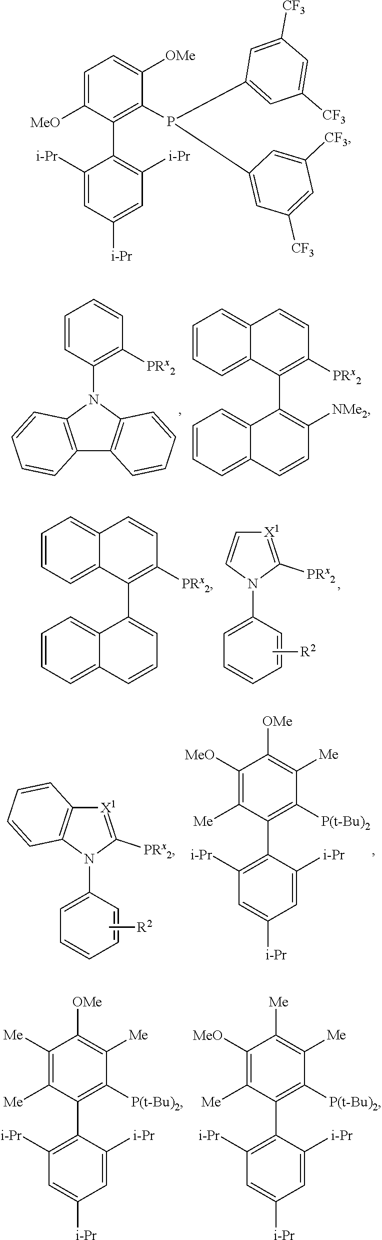 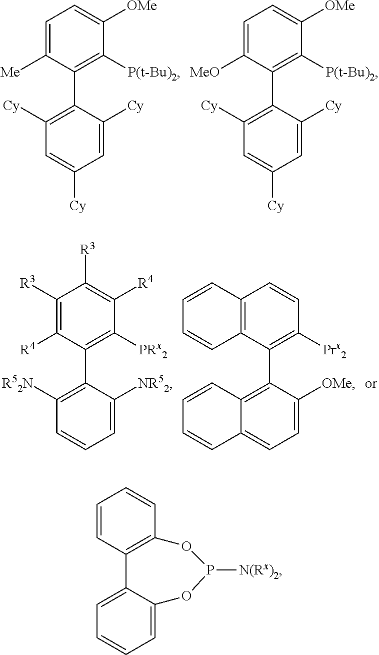
wherein
R^{x} is independently for each occurrence alkyl, aralkyl, cycloalkyl, or aryl;
X¹ is CH or N;
R² is H or alkyl;
R³ is H or alkyl;
R⁴ is H, alkoxy, or alkyl; and
R⁵ is alkyl or aryl.


SEQUENCE LISTING (5 sequences)

SEQ ID NO: 1 - protein, 11 aa, Artificial Sequence
  Description of Artificial Sequence Synthetic peptide
RSNFFLGCAGA

SEQ ID NO: 2 - protein, 14 aa, Artificial Sequence
  Description of Artificial Sequence Synthetic peptide
ACYKRSDFTCGGGS

SEQ ID NO: 3 - protein, 8 aa, Artificial Sequence
  Description of Artificial Sequence Synthetic peptide
TDEYCKSR

SEQ ID NO: 4 - protein, 14 aa, Artificial Sequence
  Description of Artificial Sequence Synthetic peptide
ITFCDLLCYYGKKK

SEQ ID NO: 5 - protein, 130 aa, Artificial Sequence
  Description of Artificial Sequence Synthetic polypeptide
GGCGGSDLGKKLLEAARAGQDDEVRILMANGADVNAYDDNGVTPLHLAAFLGHLEIVEVLLKYGADVNAADSWGTTPLHLAATWGHLEIVEVLLKHGADVNAQDKFGKTAFDISIDNGNEDLAEILQKLN